SEQUENCE LISTING (117 sequences)

SEQ ID NO: 1 - DNA, 1421 bp, Arabidopsis thaliana
gtagttagatgattacaaaggggaaatttaggttaaaagcgtttttttttattctgagtaaaatttgggaatagctttagattgtggggttacagataaagtagagctatgtgttagtaaaagtctttgtggtagtgacttgtgataatatttattgttacaggtaagtgggaagagagttgggatagttggattggggagcattggatcatttgttgctaaaagacttgaatcatttggctgtgttatctcttacaactcaaggagtcagaaacagagtagtccataccggtattactctgacattctctcgttagcagagaacaacgatgtacttgtcctctgctgctctttgacagacgaaacgcaccatattgtgaatagagaagtgatggagttgcttggtaaggatggggttgtgatcaatgtgggacgaggaaagttgattgatgagaaggagatggtcaagtgtttggttgacggtgtgattggtggtgctggtttagatgtgtttgagaatgaaccggcagttcctcaggagttgtttggtttggataatgtagtgttgtctcctcattttgctgtggctacaccagggtctttggacaatgttgcacagattgctttagctaacttgaaggcgtttttctcgaaccggcctttgctttctccggttcaattggattgagagagcgcccggtttgatcaggtagctaaattagttaagctattgtttattataatcaataattcaaaaagaaagtgtaatgaatatttgaatgtaccctgacattctctcccaaagaagaagaattaatgacgcatattatttaaataattctcccgcgttgcacatatgactaatttagtcggaacattacgattggcaatataatcataatgtttatgaataaccttttggttctaatgttattgtgaaaatactgttaaaacatgatttcatatattagtttatctttggaaacgtaaatagttgacaaacgacaatataaaaataaatgtctgctgttcaatttaactaatcattgaaaatacataaacgcacgtatatatagacattggatagagtcggtacacgtatcgtctatagaacctgctcgcacgtcaacttatactatattcaaaaacctcacttaaacaacaattgaccttttttcctaaattttattagtatttctattgaaaaaattcaatgaaatgaaacaaatcccaatcggtacggacaaaagtctccaataaaaaaggaattaaaaaaaaaaaggatagtgatccgcacgtagccaccactactgtcgttgaaaatcccctctatataagattgtctcaaattcgattacttcatcaaaaaacaaaccaaaaacaaaccctaagaataaagaaaaagaggctagaatgggtcc

SEQ ID NO: 2 - DNA, 778 bp, Arabidopsis thaliana
atggacgtttttgttgatggtgaattggagtctctcttggggatgttcaactttgatcaatgttcatcatctaaagaggagagaccgcgagacgagttgcttggcctctctagcctttacaatggtcatcttcatcaacatcaacaccataacaatgtcttatcttctgatcatcatgctttcttgctccctgatatgttcccatttggtgcaatgccgggaggaaatcttccggccatgcttgattcttgggatcaaagtcatcacctccaagaaacgtcttctcttaagaggaaactacttgacgtggagaatctatgcaaaactaactctaactgtgacgtcacaagacaagagcttgcgaaatccaagaaaaaacagagggtaagctcggaaagcaatacagttgacgagagcaacactaattgggtagatggtcagagtttaagcaacagttcagatgatgagaaagcttcggtcacaagtgttaaaggcaaaactagagccaccaaagggacagccactgatcctcaaagcctttatgctcggaaacgaagagagaagattaacgaaaggctcaagacactacaaaaccttgtgccaaacgggacaaaagtcgatataagcacgatgcttgaagaagcggtccattacgtgaagttcttgcagcttcagattaagttgttgagctcggatgatctatggatgtacgcaccattggcttacaacgggcctggacatggggttccatcacaaccttttgtctcggcttatgtga

SEQ ID NO: 3 - DNA, 3054 bp, Arabidopsis thaliana
tctaagcggctttggtctgaattttttatatacaaggcctgtctccgtttttgtaaagggaaaacagtaggatccattttagcctctgtaagtaacaatattgggcccctaaaagcccacccattttggggccagcaaaccaaggcaccctcggttccgcacgctcgctaagacgctaacctatgcatatgttgatatgttttttctcttccttttggtatgaatcttgatttgttttgatactcatgatgtacattcgtattctcttacgtattgtaaaccatcctatttcagatcacgattatatctttacatttacatttttcatttttatttctgtttgaatgttacaatttactagtagagttattcattaaaatactacaactggtatacagaaatgtaatttgagtgataaattatatgaaataattaagtaatatatgtgatatttatggatccaaacaaaaactaattactggttcattttctattttagatgtaagcaaaatgtgtaagattcaaggtatatatatatcccaatatacgtatatatgtggtactcactagctagtagctctctcacaactgtgtcttttggttttcatcagctgatcctctccaactaactatccatcttttgttttgcggttggacttggaggtaccaagaatattagcaacgtacgactcgtatggtatcatttcctttgtacaaaaagtgaatatcaaaatgcattgtattaattatatataagttagtatatggagttagttgtcctcactgtctttatctggcgcaatctcctatgccatcattccctcttcacacgtacgtgtgcacactcgatgtcacatttgtataaacacgtttgcttttagcgtgagatcatcaccatattccatttttggtgggtcagttctctttctagatagttatttgtaaggacgtgaattaaaagggatcgtcgtcacttgttgagataaaagaaaagatatatggtcagtttctgcatcttggaatcaacttaagggttgtcttaattaattttgatagaccctactttaaaaattaattagttgctttcattggccctcaataagaaaagccaaaaaagaaagaagactggtcttggaagtttgccaacacgggtaatagattaatggtgaaaagggcgaatttttttacccaaaaccctaattaagtagaagtattaatcgagagcaaaaaagagagagagagtcagtagccaaaaggaatgaatggaagaaagaaaaaggaatctctataggcagcatatattcaagtaattaattaaagtagatagatagagcaaaaggagaggttaggaggcattaattaattatttaagagcatgtggtgaatgtaaatgtttatggttgcttccctctctatacattatgtatctacctttcctaactaacaattccctaggccgtacgacgactaacaaagaaaaaaacaaaagaaactgataaagcttttgaattgtagataaatcatctgctacagttataccattatatatcttattaaagacctaagtttccttcactatacgtcttcgtccatttacgtacgtattatacggacggtttaagctactatatctatattgttaacaatgtaactgttgagatatatcttgcaataatatgtcatggtgtatgcatacgataatatgaatcaatgtttgaaatcttgacgtgcccgtgatacaataagatgatcaaaatttcaaattttgtcaaatattaaaacaacatacacatacacatgtgtccaggtggcattataaaatgtatatatggtggatatagagagagagggagatgcgtatagtgaataggaaagtaagtaataaagagagggtggaggaattggaaaggggttggaggcaaacccataaagagcattcatttccttttaaggtcgctgaaattaatgagtaacgatcggtcaatgcctctcgctgacctttttctttttttacaacaacaaataaaaataaaataaatttcgacgtctctttccgctgctgaattacatttgttgaattaattttctctgcttacgtacgtcttctaaactttctctatccgaattcttttttaactttctaacttatattcaacaactcttctttcctgcctttaccgttagtctaattgttttcctaatactgctacgtacatacccctactatactagtcagtgtattagattcgattgggattaatccaggaatatagatatcccattagtttttataaaaatattggaagaggacaagtctcaagcaatttagggttccatgtagcgctgcaatatactgttagtaactctctcttacccatatattgtatatgctaattcttatcaaatatatatatatgcttctcccagagtcccagtttcctataatcctgacgcaattatactaatagagccaagtttacataataaagtatatatgattaatagatagggtttcttattaagccatatcttaaattaagatgtgatgatagcgttttgtataagttaccaattgtttgaaagaagagatcatcacaataataaatcataagtagtagtatatagtaataaataaatacacaagtcataataagagtaatgagaggataattaaggagggaagaagaaagcagaaaatgcggttggagaattaggtgctaaaagttagttgagtccatctcagtatctaacggtcaactctctctctctctagagaaaacaattaagaaatctgacatacacatatgtctctctctctctctctctctagtctatacacacaattcaattaaagaagagacagagaagttcgtcttttttgtttttatacccttaaatcaatcatgcaattgtaacccttccttcttattctcattccttccccccctgtctacagtaatctatagcaacgccattatgtactacttttaacggataatttgctcatgtttcaatatggcttcattgtatatatgttcaagttcttctcaatcc

SEQ ID NO: 4 - protein, 223 aa, Arabidopsis thaliana
MENEAFVDGELESLLGMFNFDQCSSNESSFCNAPNETDVFSSDDFFPFGTILQSNYAAVLDGSNHQTNRNVDSRQDLLKPRKKQKLSSESNLVTEPKTAWRDGQSLSSYNSSDDEKALGLVSNTSKSLKRKAKANRGIASDPQSLYARKRRERINDRLKTLQSLVPNGTKVDISTMLEDAVHYVKFLQLQIKLLSSEDLWMYAPLAHNGLNMGLHHNLLSRLI

SEQ ID NO: 5 - protein, 298 aa, Arabidopsis thaliana
MALVNDHPNETNYLSKQNSSSSEDLSSPGLDQPDAAYAGGGGGGGSASSSSTMNSDHQQHQGFVFYPSGEDHHNSLMDFNGSSFLNFDHHESFPPPAISCGGSSGGGGFSFLEGNNMSYGFTNWNHQHHMDIISPRSTETPQGQKDWLYSDSTVVTTGSRNESLSPKSAGNKRSHTGESTQPSKKLSSGVTGKTKPKPTTSPKDPQSLAAKNRRERISERLKILQELVPNGTKVDLVTMLEKAISYVKFLQVQVKVLATDEFWPAQGGKAPDISQVKDAIDAILSSSQRDRNSNLITN

SEQ ID NO: 6 - DNA, 897 bp, Arabidopsis thaliana
atggcactcgttaatgaccatcccaacgagaccaattacttgtcaaaacaaaattcctcctcttccgaagatctctcctcgccgggactggatcagccagatgcagcttatgccggtggaggaggaggaggaggctcggcttcgagcagtagcacgatgaattcagatcatcaacaacatcaggggtttgtattttacccatccggtgaagatcatcacaactctttgatggatttcaacggatcatcatttcttaactttgatcatcacgagagctttcctcctccagccataagctgtggtggtagtagcggtgggggcggcttctccttcttggagggcaacaacatgagctacggcttcacaaactggaatcatcaacatcatatggatattattagccctagatccaccgaaactccccaaggccagaaagactggttatattctgattcaactgttgtaaccactggttctagaaacgagtctctttcgcctaaatccgctggaaacaaacgttctcacacgggagagagcactcaaccgtcgaagaaactgagtagcggtgtgaccggaaagaccaagcctaagccaacaacttcacctaaagatccacaaagcctagcagccaagaatcgaagagaaaggataagtgaacgtctcaagatattgcaagaacttgttcccaatggcaccaaggttgatttggtgacaatgcttgaaaaggctattagttatgtcaagttccttcaagtacaagttaaggtattagcgaccgatgagttttggccggctcaaggaggaaaagctcctgacatttctcaagttaaagacgccattgatgccattctctcctcatcacaacgagacaggaattcgaatctgatcaccaattaa

SEQ ID NO: 7 - protein, 307 aa, Arabidopsis thaliana
MSLINEHCNERNYISTPNSSEDLSSPQNCGLDEGASASSSSTINSDHQNNQGFVFYPSGETIEDHNSLMDFNASSFFTFDNHRSLISPVTNGGAFPVVDGNMSYSYDGWSHHQVDSISPRVIKTPNSFETTSSFGLTSNSMSKPATNHGNGDWLYSGSTIVNIGSRHESTSPKLAGNKRPFTGENTQLSKKPSSGTNGKIKPKATTSPKDPQSLAAKNRRERISERLKVLQELVPNGTKVDLVTMLEKAIGYVKFLQVQVKVLAADEFWPAQGGKAPDISQVKEAIDAILSSSQRDSNSTRETSIAE

SEQ ID NO: 8 - DNA, 924 bp, Arabidopsis thaliana
atgtcactcattaacgaacattgcaatgagcgtaattacatctcaaccccaaattcttcagaagatctctcttcaccacagaattgcggattagacgaaggagcttcagcttcaagcagtagcaccataaattctgatcatcaaaataatcaagggtttgtgttttacccttccggggaaaccattgaagatcataattctttgatggatttcaatgcttcatcattcttcacctttgataatcaccgaagccttatctctcccgtgaccaacggtggtgccttcccggtcgtggacgggaacatgagttacagctatgatggctggagtcatcatcaagtggatagtattagccctagagtcatcaaaactccaaatagctttgaaacaacgagcagttttggattgacttcaaactccatgagtaaaccggccacaaaccatggaaatggagactggttatactctggttcaactattgtaaacatcggttcaaggcacgagtccacgtcccctaaactggctggcaataaacggcctttcacgggagagaacacacaactttcaaagaagccgagtagcggtacgaatggaaagatcaagcctaaggcaacaacttcacctaaagatccacaaagcctagcagccaagaaccgaagagaaaggataagcgaacgcctcaaggtattgcaagaacttgtaccgaatggtaccaaggtggatttggtaactatgcttgagaaagcaattggctatgtaaagtttcttcaagtacaagttaaggtacttgcagccgatgagttttggccggcacaaggagggaaagctccggacatttctcaagttaaagaagctattgacgcaatcctctcatcatcacaacgagatagtaactcaactagagaaacaagtatagcagaataa

SEQ ID NO: 9 - protein, 352 aa, Arabidopsis thaliana
MEAMGEWSNNLGGMYTYATEEADFMNQLLASYDHPGTGSSSGAAASGDHQGLYWNLGSHHNHLSLVSEAGSFCFSQESSSYSAGNSGYYTVVPPTVEENQNETMDFGMEDVTINTNSYLVGEETSECDVEKYSSGKTLMPLETVVENHDDEESLLQSEISVTTTKSLTGSKKRSRATSTDKNKRARVNKRAQKNVEMSGDNNEGEEEEGETKLKKRKNGAMMSRQNSSTTFCTEEESNCADQDGGGEDSSSKEDDPSKALNLNGKTRASRGAATDPQSLYARKRRERINERLRILQNLVPNGTKVDISTMLEEAVHYVKFLQLQIKLLSSDDLWMYAPIAFNGMDIGLSSPR

SEQ ID NO: 10 - DNA, 1059 bp, Arabidopsis thaliana
atggaagccatgggagaatggagcaacaacctcggaggaatgtacacttatgcaaccgaggaagccgatttcatgaaccagcttctcgcctcttatgatcatcctggcaccggctcatcctccggcgcagcagccagtggtgaccaccaaggcttgtattggaaccttggttctcatcacaaccaccttagcctcgtgtctgaagccggtagcttctgtttctctcaagagagcagcagctacagcgctgggaacagcggatattacaccgttgttccacccacggttgaagagaaccaaaatgagacaatggactttgggatggaagatgtgaccatcaatacaaactcataccttgttggtgaggagacaagtgagtgtgacgttgagaaatactcttctggaaagactcttatgcctttggaaaccgtagtggagaaccacgatgacgaggaaagcttgttgcaatctgagatctctgtgactactacaaaatctctcaccggctccaaaaagagatcccgtgccacatctactgataaaaacaagagagcaagagtgaataagagggcccagaagaacgtagagatgagtggggataacaatgaaggagaagaggaagaaggagagacgaagttgaagaaaagaaagaatggggcaatgatgagtagacagaactcaagcaccactttctgtacggaggaagaatcaaactgcgctgatcaagacggtggaggagaagactcatcctctaaggaagatgatccctcaaaggccctcaacctcaatggtaaaacaagagccagtcgtggtgcagccaccgatcctcaaagcctctatgcaaggaaaagaagagaaaggattaacgagagactaaggattttacaaaatctcgtccccaatggaacaaaggtcgatattagtacaatgcttgaggaagcagttcattacgtcaaatttttgcagctccaaattaagttattgagctctgatgatctatggatgtatgcgccgattgctttcaatgggatggacattggtctcagctcaccgagatga

SEQ ID NO: 11 - protein, 519 aa, Arabidopsis thaliana
MEAMGEWSTGLGGIYTEEADFMNQLLASYEQPCGGSSSETTATLTAYHHQGSQWNGGFCFSQESSSYSGYCAAMPRQEEDNNGMEDATINTNLYLVGEETSECDATEYSGKSLLPLETVAENHDHSMLQPENSLTTTTDEKMFNQCESSKKRTRATTTDKNKRANKARRSQKCVEMSGENENSGEEEYTEKAAGKRKTKPLKPQKTCCSDDESNGGDTFLSKEDGEDSKALNLNGKTRASRGAATDPQSLYARVDISTMLEEAVQYVKFLQLQIKRLLAIGTNHRNRSIPLWTARNRQISKAHSRKRLRLRAVAKIIWSDEMTRFLLELITLEKQAGNYRGKSLIEKGKENVLVKFKKRFPITLNWNKVKNRLDTLKKQYEIYPAKLRSHPLRFIPLLDVVFRDETVVVEESWQPRRGVHRRAPVLDLSDSECPNNNGDEREDLMQNRERDHMRPPTPDWMSQTPMENSPTSANSDPPFASQERSSTHTQVKNVSRNRKRKQNPADSTLDRIAATMKKI

SEQ ID NO: 12 - DNA, 987 bp, Arabidopsis thaliana
atggaagccatgggagaatggagcaccggcctaggcggaatatatacagaggaagctgactttatgaatcagctccttgcctcctatgagcaaccttgtggcggttcatcttcagagacaaccgccacactcacggcctaccaccaccagggttctcaatggaatggtggcttttgcttctctcaggagagcagtagttatagtggttactgcgcggcgatgccacggcaagaagaagataacaatgggatggaggacgcgacaatcaacacgaacttgtaccttgttggtgaagagacaagtgaatgtgatgcgacggaatactccggtaaaagcctcttgcctttggagactgtcgcagaaaaccacgaccatagtatgctacagcctgagaactccttgaccacgaccactgatgagaaaatgttcaaccaatgtgagagttcaaagaagaggacgcgtgccacaacaactgataagaacaagagagccaacaaggcacgaaggagccagaaatgcgtagagatgagtggcgaaaatgaaaatagcggcgaagaagaatatacggagaaggctgcggggaagagaaagaccaaaccacttaagccgcaaaagacttgttgttcggatgacgaatcaaacggtggagacactttcttgtccaaagaagatggcgaggactctaaggctctcaacctcaacggcaagactagggccagccgcggcgcggccacagatcctcaaagcctttacgcaaggaaaagaagagagaggataaacgagaggctaaggattttgcaacatctcgtccctaatggaacaaaggttgatattagcacgatgttggaagaagcagtacaatacgtcaaatttctacagctccaaattaagttattgagctctgatgatctatggatgtatgcgcctattgcttacaacggaatggacattggccttgacctaaaactcaatgcactgaccagatga

SEQ ID NO: 13 - protein, 223 aa, Arabidopsis thaliana; identical to SEQ ID NO: 4

SEQ ID NO: 14 - DNA, 672 bp, Arabidopsis thaliana
atggagaatgaagcttttgtagatggtgaattggagtctcttttggggatgttcaactttgatcaatgttcatctaacgaatcgagcttttgcaatgctccaaatgagactgatgttttctcttctgatgatttcttcccatttggtacaattctgcaaagtaactatgcggccgttcttgatggttccaaccaccaaacgaaccgaaatgtcgactcaagacaagatctgttgaaaccaaggaagaagcaaaagttaagctcggaaagcaatttggttaccgagcctaagactgcttggagagatggtcaaagcctaagcagttataatagttcagatgatgaaaaggctttaggtttagtgtctaatacatcaaaaagcctaaaacgcaaagcgaaagccaacagagggatagcttccgatcctcagagcctatacgctaggaaacgaagagaaaggataaacgataggctaaagacattgcagagcctagttcctaatgggacaaaggtcgatataagcacaatgctggaagatgctgtccattacgtgaagttcctgcagcttcaaatcaagctcttgagttcagaagatctatggatgtatgcacctcttgctcacaatggtctgaatatgggactacatcacaatcttttgtctcggcttatttaa

SEQ ID NO: 15 - protein, 67 aa, Arabidopsis thaliana
TSPKDPQSLAAKNRRERISERLKILQELVPNGTKVDLVTMLEKAISYVKFLQVQVKVLATDEFWPAQ

SEQ ID NO: 16 - protein, 67 aa, Arabidopsis thaliana
TSPKDPQSLAAKNRRERISERLKVLQELVPNGTKVDLVTMLEKAIGYVKFLQVQVKVLAADEFWPAQ

SEQ ID NO: 17 - protein, 67 aa, Physcomitrella patens
GSANDPQSIAARVRRERISERLKVLQALIPNGDKVDMVTMLEKAISYVQCLEFQIKMLKNDSLWPKA

SEQ ID NO: 18 - protein, 67 aa, Physcomitrella patens
GSANDPQSIAARVRRERISERLKVLQALIPNGDKVDMVTMLEKAITYVQCLELQIKMLKNDSIWPKA

SEQ ID NO: 19 - protein, 67 aa, Physcomitrella patens
GSATDPQSVYARHRREKINERLKSLQNLVPNGAKVDIVTMLDEAIHYVKFLQNQVELLKSDELWIYA

SEQ ID NO: 20 - protein, 67 aa, Physcomitrella patens
GSATDPQSVYARHRREKINERLKNLQNLVPNGAKVDIVTMLDEAIHYVKFLQTQVELLKSDEFWMFA

SEQ ID NO: 21 - protein, 67 aa, Physcomitrella patens
GSATDPQSVYARHRREKINERLKTLQHLVPNGAKVDIVTMLDEAIHYVQFLQLQVTLLKSDEYWMYA

SEQ ID NO: 22 - protein, 67 aa, Physcomitrella patens
GSATDPQSVHARARREKIAERLRKLQHLIPNGGKVDIVTMLDEAVHYVQFLKRQVTLLKSDEYWMYA

SEQ ID NO: 23 - protein, 67 aa, Physcomitrella patens
GSATDPQSVYARHRREKINERLKTLQRLVPNGEQVDIVTMLEEAIHFVKFLEFQLELLRSDDRWMFA

SEQ ID NO: 24 - protein, 67 aa, Arabidopsis thaliana
GAATDPQSLYARKRRERINERLRILQNLVPNGTKVDISTMLEEAVHYVKFLQLQIKLLSSDDLWMYA

SEQ ID NO: 25 - protein, 67 aa, Arabidopsis thaliana
GAATDPQSLYARKRRERINERLRILQHLVPNGTKVDISTMLEEAVQYVKFLQLQIKLLSSDDLWMYA

SEQ ID NO: 26 - protein, 67 aa, Arabidopsis thaliana
GTATDPQSLYARKRREKINERLKTLQNLVPNGTKVDISTMLEEAVHYVKFLQLQIKLLSSDDLWMYA

SEQ ID NO: 27 - protein, 67 aa, Arabidopsis thaliana
GIASDPQSLYARKRRERINDRLKTLQSLVPNGTKVDISTMLEDAVHYVKFLQLQIKLLSSEDLWMYA

SEQ ID NO: 28 - protein, 762 aa, Physcomitrella patens
MAGPAGALWSTCDPQPIQQAEIFSGPDNQAGLMSFHVDTPFHWGSEPWALHSRSDDIALMSPSLVHDISPYDSVLHLSGVSGDVQDLVCGNPKFRQSGQWGQSEFSYSVQDNMQDLLTNQFIPYNTSSLGLNHLSPNFTDLDCAPVYNDTKAFGTVTHNRAVPSTNTQSAQHGSSSMVSSNRPITSTASPTTQYGGPRTPSQTTQYGGSSMVTNSMEMFASAAPQGIMTTSGLSGGCNSDLMHLPKRQHAHSLPPTTGRDLTASEVVSGNSISNISGVGSFNSSQKSSASVMMSPLAASSHMHKAAAVSEELKMASFNPGPFVPTQKKQQHEQQDTMTSNRIWADKNNLGKISSSPIPIMGFEQSQQQSMSNSSPVTSLGFEQRQKMSMGSSPSITIIGFEQRQKQPMSSSSPISNMVFEPRQKQPMSSSSPISNIVFEQRQLPTVGSSPPISISGFEPKKQPSLSNSPPLSNLGFEQRLQPMSNASPISNLPFEQQRQQATMSNTRSAEPDSVESTTKWPLRMDGAIGGCAGLPSSQKAPVIMQPETGTMKCPIPRTMPSNAKACPAVQNANSVNKRPLTVDDKDQTGSMNKKSMQKFLGPQGCSRLESISALAHQKVSQSTTSGRALGPALNTNLKPRARQGSANDPQSIAARVRRERISERLKVLQALIPNGDKVDMVTMLEKAISYVQCLEFQIKMLKNDSLWPKALGPLPNTLQELLELAGPEFAGIDGKNTEESSEKPKKSALEVIELDGNQPSAD

SEQ ID NO: 29 - DNA, 2289 bp, Physcomitrella patens
atggcaggtccagcaggagctttatggagtacttgtgatccacagcctattcaacaggcagagatatttagtggtcctgacaaccaagctggtttgatgtcttttcatgtggataccccgttccattggggatctgaaccatgggctctccactctcggtcagatgacatcgccttgatgtccccctcgcttgttcacgacatatcaccttatgattctgtcttgcatctttccggagtgtctggggatgtgcaagatttagtttgcgggaatcccaaatttcgccaaagtgggcaatgggggcagagcgagttttcatactctgttcaggacaacatgcaagatctcctaaccaaccagttcataccgtacaacacatcttcattgggtttaaatcatctctccccgaatttcaccgacttggattgcgcaccggtatacaatgataccaaggcttttggcactgttacacacaacagggcagtcccgagcactaatacccagagtgctcagcacgggagttcgtctatggtttcaagtaacaggccaatcactagcacagcttctcctactactcagtatggaggtccgaggactccatcccaaaccacccagtacgggggttcatctatggttaccaactcgatggaaatgtttgcttcagctgcacctcagggtattatgactacatctggcttgagtggcggttgcaactcagacttgatgcatctgccgaagcgccagcatgctcactctcttcctcctaccactggcagagatttaactgcatctgaagtggtatctggaaattcgatatcaaacatttccggggttggatcttttaacagcagccagaaaagcagtgcatccgtgatgatgtctcctttagctgcttcttctcacatgcacaaggctgctgctgtatctgaagaacttaagatggcaagtttcaaccctggtccattcgtacctacgcagaaaaagcagcaacatgagcagcaggatacgatgacctctaatcgtatatgggcggataagaacaacttgggaaaaattagttcatcgcccattccgatcatggggtttgagcagagtcaacagcaatccatgagcaattcctcccctgttaccagtttggggtttgagcaaaggcaaaaaatgtccatgggtagctctccctccatcacgatcattggatttgagcaaagacagaagcaacctatgagtagttcttcccccatttcaaacatggtttttgaaccaagacaaaaacagccaatgagtagctcttctcctatctctaatattgtctttgagcaaagacaactcccaactgtgggtagctctcctccgatttcaatctcaggatttgagccaaagaaacaaccatctttgagcaattctcctcccctctctaatctgggttttgagcaaaggctacaacccatgagtaatgcatctcctatttccaacttaccctttgagcaacaaagacaacaagcaaccatgagtaacaccagatctgcagaacccgattctgtcgagtctaccacgaagtggcccttgcggatggatggtgccataggtggatgtgctggcttaccaagcagtcagaaagctcctgttatcatgcagcctgagactgggactatgaagtgtcctattccgaggaccatgcccagcaatgctaaggcttgcccagctgtgcagaatgctaattccgtaaacaagcgccctcttacggttgatgacaaggaccaaactggatcgatgaataagaagtcgatgcaaaagtttttgggacctcaaggttgtagcagacttgaaagtatcagtgctttagctcaccaaaaagtgagtcaaagtacaacaagcggtcgtgctctagggcctgctttgaacaccaatctcaagcctcgtgcacgccaagggagtgccaatgatccgcagagcattgctgctagggtgcgaagagaaagaataagtgagcggctcaaagttttgcaagccttgatacctaacggtgataaagtggatatggtcaccatgctggagaaggctatcagctacgtgcagtgtttggaatttcagattaagatgttaaaaaatgactctttgtggcctaaggcgcttggccctctaccgaacactttgcaagagcttctcgaacttgctgggccagagtttgccggcatagatggcaagaatactgaggagtcgtcagagaaaccgaagaaatctgctcttgaagtaattgagttggacggcaatcagccttctgctgactaa

SEQ ID NO: 30 - protein, 173 aa, Physcomitrella patens
MNKKPMQKALGPQGCSRLESISALAHQKVSQSASGRALGPALNTNLKPRARQGSANDPQSIAARVRRERISERLKVLQALIPNGDKVDMVTMLEKAITYVQCLELQIKMLKNDSIWPKALGPLPNTLQELLELAGPEFSGTESKNVEEPPAKPKKSAPDVIEFDGNQPSADKE

SEQ ID NO: 31 - DNA, 522 bp, Physcomitrella patens
atgaataagaagcctatgcaaaaagctttgggacctcaaggatgcagcaggctagaaagcatcagtgctttagctcatcaaaaagtgagtcagagtgcaagtggtcgtgcactagggcctgctctgaacaccaacctcaagcctcgtgctcgtcaagggagtgccaatgacccacagagcattgccgctagggttcgaagagaaaggataagtgagcggctgaaagttttgcaagccttgatacctaatggtgataaggtagatatggtgaccatgctggagaaggctatcacctacgtgcagtgtctggaactccagattaagatgttaaagaatgattctatctggcccaaggcgcttggacctctaccaaacactcttcaagagcttctggagcttgctggaccagaattttctggaacggaaagcaagaatgtagaggagcccccagcgaagccaaagaaatcagctcctgacgttattgagttcgacggcaatcaaccttctgccgacaaagagtag

SEQ ID NO: 32 - protein, 67 aa, Physcomitrella patens; identical to SEQ ID NO: 21

SEQ ID NO: 33 - DNA, 201 bp, Physcomitrella patens
ggttcagcgactgatccgcagagtgtatatgccaggcatagaagggagaagatcaacgagcgcttgaagacattacagcacttggtaccaaatggagctaaggtagacatcgtgaccatgcttgacgaagccattcactacgtccaatttctgcagctccaagtgacgctgttgaagtcggatgaatattggatgtacgcc

SEQ ID NO: 34 - protein, 538 aa, Physcomitrella patens
MTDLISILESSGSSREEMCPVAVPSSVASSCERLIWEGWTAQPSPVEESTTSKLLPKLLPELETSSYSALTLQQPDALSSILSVLHPFSHYSSASLELARNPDWSLKSSNPLRESSSEAGIRTSSFEGLYSGQHTTKKIHLGVIPYHLSEDQRQCAVSPPENECRLLSANSSGSLHWWHSIGPESPSSTLAFHNIGIQHSTFEKCEPRGQSHSSWPAASGTSPTVQYFHAHSADNEGVEVVKQDDSQISKALATYQPHGDHSLVLNSDRIASTTSHSEDPCGPKPGRRPAASYDTEMILSPSESFLTTPNMLSTLECVISGASNISDQYMNFVREPQEQRLSSISDLSLIPDSHADPHSIGFISGTFRTDSHGTGIRKNRIFLSDEESDFLPKKRSKYTVRGDFQMDRFDAVWGNTGLRGSSCPGNSVSQMMAIYEFGPALNRNGRPRVQRGSATDPQSVHARARREKIAERLRKLQHLIPNGGKVDIVTMLDEAVHYVQFLKRQVTLLKSDEYWMYATPTSYRSKFDDCSLVPGENN

SEQ ID NO: 35 - DNA, 1617 bp, Physcomitrella patens
atgaccgatctgatttcgatcttggagtcatcagggtcatcacgagaggagatgtgccctgttgctgtgccaagctccgtggcttcttcttgtgaaaggttgatatgggaggggtggactgcacaaccatctcctgtcgaagaaagcaccaccagcaagttacttccaaagctacttccagagctcgagacatcatcctactctgcactcacccttcagcaacctgatgcgctctccagcatactttcagtcctccaccctttttctcattacagttcggccagtttagaactcgctcgcaatcctgactggagcttgaaatcttcaaatcctctgcgggaaagcagctcggaggctggcatccgaacctcatctttcgaaggcttgtactctggtcagcacaccaccaaaaagattcatttgggggtcataccctaccacttgtccgaagatcagcgccagtgcgctgtcagtcctccggaaaatgagtgccgcctactgtctgcaaattcctctggatcccttcactggtggcattccataggccccgagtctccttcctctactcttgcattccataatattgggatccaacactctaccttcgaaaagtgtgagcctaggggccagtcgcactcatcatggccagcggccagcggcacgtcgccaacagttcaatactttcatgcccattctgcagataatgaaggtgtcgaggtcgtcaagcaagatgactcgcagatatccaaggctctggcgacctatcaaccccacggcgaccatagtctcgtgctaaattcagaccgcattgcaagcacaaccagccactcagaagatccttgcggccctaaacctggacgcagaccagctgcatcatacgacaccgagatgattcttagcccaagtgagagtttcttgacaactcccaatatgttatcaacgttggagtgcgtaatatccggtgcaagtaacatatctgatcagtatatgaacttcgtcagagaaccgcaggagcaaaggctgtcctctatctccgatctgtcccttattcctgacagccacgcggatccgcacagtatcggatttatctctgggacctttagaacagactcccacggaactggaataagaaagaaccgcatctttctcagtgatgaggaatccgacttcttgcctaagaagcgatccaagtacacggtccgcggcgattttcagatggatcgcttcgacgcagtttgggggaataccggtcttcggggatctagctgtcctggaaattcagtatcccagatgatggcgatttacgaattcggacccgcactgaacaggaacggcaggccgcgagtacaacgtggttcggcgactgatccgcagagtgtacacgccagggcgcggagggagaaaatcgccgagcgcttgagaaagttgcagcacctcattccaaacggcgggaaggtggacatcgtaaccatgctcgacgaagccgttcactatgttcagtttttgaagcgacaagttacgcttctgaaatccgacgagtattggatgtacgccacgccgacctcgtaccggagcaaattcgacgactgcagtctggttcccggcgagaacaactga

SEQ ID NO: 36 - protein, 539 aa, Physcomitrella patens
MVQLYMSSVEEQRETMVQPYVSSMDSGSTSGRQTPSCVVQQGSNTFETSNLWEEWTQASNGDDTVSTSNFLPEISSFTSSRLSFQQSDSLTTWMSGFPPLSQTALSPDLSHSSDPVDHPPAFMQEGLGPGDSILDYSPALTEMYPKSSSKHNSSDCLPYPAASAPDKKMTDHELGSAISLAYDRGTVSRQLLRALGPLSPSSPLALQNGLQNPLGDPWDASPSAMPWPMATTGHAYGPGATRTSIPDHLANAINHLEGIAPSSASHASKPRHTDIFIAPNGTFDSTPGGWTPQYYDGSVTTDESVKAMKLIASLREAGHAEATIGFCTESKPSFLRGGDRTTSPVDSFFGKCVGAKTSIKQACSGKHPLELEEIVDSENSELNPTQLKRSKLFENHPNALWSDQSMNGRELRSYSHLVGSSLTASQPMDIIAIGPALNTDGKPRAKRGSATDPQSVYARHRREKINERLKSLQNLVPNGAKVDIVTMLDEAIHYVKFLQNQVELLKSDELWIYATPNKYNGMDISDLSDMYLQELESRA

SEQ ID NO: 37 - DNA, 1620 bp, Physcomitrella patens
atggtgcagttatacatgtcctcagttgaagagcagcgggaaacaatggtacagccatacgtctcaagcatggactcaggctcaacgtcggggcgccagacgccatcttgcgtcgttcagcagggaagtaacacatttgagacttcgaatctgtgggaggaatggacgcaagcatcgaacggcgacgatacagtctccaccagcaatttcctccccgaaatcagttccttcacgtcgagtcgtctctccttccagcaaagcgactctctcaccacttggatgtcagggttccctcccctctcccaaactgccttgagcccggatcttagtcactcctccgaccccgtggatcatcccccagcattcatgcaggagggtttaggccccggtgattctattctggactattcccccgctctcacagagatgtacccgaaaagtagctccaaacataattcctcggattgtttaccttaccctgcggccagtgcaccagacaaaaaaatgactgatcacgaactaggttcggctatttccctcgcgtatgatagaggcaccgtttcccgccagcttcttcgagccttgggcccattgtcgccttcatcgcctctagcattgcagaatgggctgcaaaacccgcttggggacccctgggatgcttctccatctgcaatgccgtggccaatggcaacaaccggtcatgcttatggaccaggcgccaccaggacttctattccagatcacttagcaaatgcaattaatcacctggagggcattgcaccgtccagtgccagtcatgcatcgaaacctcgtcacactgatattttcattgcacccaatggcacgttcgattcgacgccgggaggttggacaccgcagtattacgatgggtccgtgacgacagatgagtctgtgaaggcgatgaagctgattgcgtccctacgtgaagcaggccacgcagaggctacaattggattctgtacagagagcaagcctagttttctcaggggtggggacagaacaacctcgccagtggacagcttcttcggcaaatgtgtaggggccaaaacgagtataaagcaagcctgttctgggaaacaccctcttgaacttgaggagatcgttgatagtgaaaacagtgaattaaatcccacccagctcaaacgctctaaactttttgagaatcatccgaatgccttgtggagcgatcagagtatgaatggaagagaactgagatcgtactctcatttggttggcagcagtcttactgcatcgcagcccatggacataattgcaattggcccagcgctcaacactgatggcaaaccacgagcaaagcggggttcagcaaccgatcctcagagtgtttacgctagacataggagagaaaaaatcaacgaacgattgaagagtttacaaaacctagtacctaatggagccaaggttgacatagtaaccatgctggacgaagctatacattacgtcaaatttttacaaaatcaagttgagctgctgaagtccgacgagttgtggatttacgcaacaccaaataagtacaacggcatggacatttccgacctctctgacatgtatttgcaggagctggagtcacgtgcgtga

SEQ ID NO: 38 - protein, 538 aa, Physcomitrella patens
MVRFNYMYPVQEQLEAMTDQHTPSMDSVSSAGEKTSSCIVQQGGNASETSNLWEEWTQGSNGDDSVSTSNFLPELNSSTSSRLAFHQSDILSTWISGYHPLSQSSLSSEFSHTSDRENHPPAFMQEGLIPSGLILDSDPALTDIYTRSSSSDSLPYPTARIMDKALTDHELESAVPLAYEKGCVPPQVLRNLGPLSPSSPLAFQNGLLNPLRDPWDSCPSALPWSNVTTASQTYGQVTTRTFIPDHSASAIDKLEAVATITAGYGASKPQHTDVFIEPNGTFQSTPAGWAPQFYDGSEATGLLVKPMRAIASLGEAGCGEATSEFCTKTKPGLLKGGDTITSPVGSLLGDCKKAESSMKQVWPGKHRLELVELVDGEDTKSSPTQLKRPKHSTDYANVLLSDHILKGAELRSYFHSGDVGLNASQAMDIIVIGPALNTNGKPRAKRGSATDPQSVYARHRREKINERLKNLQNLVPNGAKVDIVTMLDEAIHYVKFLQTQVELLKSDEFWMFANPHNYNGIDISDPSSMHSPELESNI

SEQ ID NO: 39 - DNA, 1617 bp, Physcomitrella patens
atggtgcggtttaactacatgtacccggttcaagagcagctggaagccatgacggaccaacacaccccaagcatggattcggtctcgtcggccggagagaagacatcctcttgcatcgtccagcagggaggaaatgcatccgaaacttcaaacttgtgggaagaatggacacaagggtcgaacggcgacgattctgtctctaccagcaacttcctccccgaactgaattcctccacctccagtcgtctcgcattccaccaaagcgacattctttccacttggatctcaggctaccacccactctcgcaaagcagcctgagttccgaattcagccacacctccgaccgcgagaatcaccccccagcattcatgcaagagggtttaatccccagtggtttaattcttgactctgatcctgctctcacagatatttatacgagaagcagctcctcggactctttgccataccccacggctaggatcatggacaaagcattgaccgatcacgagcttgagtctgctgtcccacttgcatatgaaaaaggctgcgttcctccccaggttctgcgtaacctagggccattgtcaccttcttcgcctctggcattccagaatggactgctaaaccccctcagggacccttgggattcgtgtccatctgcattgccatggtcaaatgtgaccacagccagccagacttacggtcaagtgacaaccaggactttcattccagatcactctgcaagtgcaatcgacaagttggaggccgtcgcaacgatcactgccggatacggcgcgtcgaaaccacaacatactgacgtcttcatagaacccaacgggacgtttcagtcgactccggcagggtgggcaccgcagttttacgatggatccgaggcgacgggcctgttggtcaagccaatgagggccatcgcatctctgggtgaagccggctgtggggaggccactagtgaattctgcacaaagaccaagccaggacttctcaaaggtggggacacaataacctcgccggtgggtagcctgttgggcgattgcaaaaaagctgagtcaagtatgaagcaagtttggcctggaaaacaccgtcttgaactcgtggaactagtcgatggtgaagacaccaaatcaagtcccacccagctcaaacggccgaaacattctacggattatgcgaatgtcctgttgagcgatcatattctgaaaggagcggagctgcggtcctacttccattctggtgatgttggtctaaatgcatctcaagcgatggacattattgtaattggcccagccttgaatactaatggcaagccgcgagctaaacggggttcagccaccgatccccagagtgtgtacgctagacataggcgagaaaaaatcaacgaacgactgaagaatttacaaaatctcgtgccaaatggagccaaggttgacattgtgaccatgctagacgaagccatacactacgtcaaattcttgcaaactcaagttgagctgctgaaatccgacgagttctggatgttcgcaaatccacacaactacaacggcatagatatctccgatccctctagcatgcattcgccggagctggagtcgaatatttag

SEQ ID NO: 40 - protein, 67 aa, Physcomitrella patens; identical to SEQ ID NO: 23

SEQ ID NO: 41 - DNA, 201 bp, Physcomitrella patens
gggtcagctactgatcctcagagtgtgtacgcaaggcatcgccgggagaagattaacgagcgcctaaagacattgcagcggttggttcctaacggagaacaggtcgacattgtgaccatgctggaagaagccattcactttgtcaaatttttggagttccaactggagctgttgcgatccgatgatcgctggatgttcgcc

SEQ ID NO: 42 - protein, 80 aa, Selaginella moelendorfii
LNTNLKPRAKQGCANDPQSIAARQRRERISDRLKILQELIPNGSKVDLVTMLEKAINYVKFLQLQVKVLMNDEYWPPKGD

SEQ ID NO: 43 - DNA, 240 bp, Selaginella moelendorfii
ctcaacactaatcttaagccgcgagcaaagcaaggttgtgctaatgatccacaaagcattgctgccagacaacgaagagaacggataagtgaccggcttaaaatcctgcaggagctcataccaaatggatccaaggtcgatctggtaaccatgctggagaaggccatcaactacgtcaagttcttgcaattgcaagtcaaagttcttatgaacgatgagtattggccaccaaagggagat

SEQ ID NO: 44 - protein, 80 aa, Selaginella moelendorfii; identical to SEQ ID NO: 42

SEQ ID NO: 45 - DNA, 240 bp, Selaginella moelendorfii
ctcaacactaatcttaagccgcgagcaaagcaaggttgtgctaatgatccacaaagcattgctgccagacaacgaagagaacggataagtgaccggcttaaaatcctgcaggagctcataccaaatggatccaaggtcgatctggtaaccatgttggagaaggccatcaactacgtcaagttcttgcaattgcaagtcaaagttcttatgaacgatgagtattggccaccaaagggagat

SEQ ID NO: 46 - protein, 76 aa, Selaginella moelendorfii
LNTNFKPRARQGSANDPQSIAARHRRERISDRLKILQELVPNSTKVDLVTMLEKAINYVKFLQLQVKVLTSDDYWP

SEQ ID NO: 47 - DNA, 228 bp, Selaginella moelendorfii
ctcaacaccaatttcaagcctcgagccaggcagggaagcgccaatgatccccagagcatcgctgctagacatcgccgggagaggatcagtgacaggctcaagatcttgcaagagctcgttccaaacagcacaaaggttgatctagtgacgatgctggagaaggccatcaattacgtcaagttcctccagctgcaagttaaggtgcttacgtcggacgactactggcca

SEQ ID NO: 48 - protein, 76 aa, Selaginella moelendorfii; identical to SEQ ID NO: 46

SEQ ID NO: 49 - DNA, 228 bp, Selaginella moelendorfii
ctcaacaccaatttcaagcctcgagccaggcagggaagcgccaatgatccccagagcatcgctgctagacatcgccgggagaggatcagtgacaggctcaagatcttgcaagagctcgttccaaacagcacaaaggttgatctagtgacgatgctggagaaggccatcaattacgtcaagttcctccagctgcaagttaaggtgcttacgtcggacgactattggcca

SEQ ID NO: 50 - protein, 80 aa, Selaginella moelendorfii
LNTDGKPRAKRGSATDPQSIYARQRRERINERLRALQGLVPNGAKVDIVTMLEEAINYVKFLQLQVKLLSSDEYWMYAPT

SEQ ID NO: 51 - DNA, 240 bp, Selaginella moelendorfii
ctaaacaccgacggaaagccacgcgcaaagcgtggatctgccacggacccgcaaagcatctacgctcggcaaagaagagaaaggatcaacgagcgtttgagagcgctacaaggactcgtaccaaacggagcgaaggttgacattgtgacgatgctcgaggaagccatcaactatgtcaagtttttgcagctgcaagtaaagctgctcagctcggacgagtattggatgtacgcccccaca

SEQ ID NO: 52 - protein, 80 aa, Selaginella moelendorfii
LNTNGKPRAKRGSATDPQSVYARHRRERINERLKTLQHLVPNGAKVDIVTMLEEAIHYVKFLQLQVNMLSSDEYWIYAPT

SEQ ID NO: 53 - DNA, 240 bp, Selaginella moelendorfii
ctcaacacgaatggcaagcccagagcaaagcgtggatctgcaacagatccccaaagcgtttacgcaaggcaccggagagagaggatcaacgagaggctcaaaactttacaacaccttgttccaaatggtgcaaaggttgacatagtgacaatgcttgaagaagcaatacattacgtgaagtttctacagctgcaagtcaacatgttaagctctgatgagtactggatttatgcacccaca

SEQ ID NO: 54 - protein, 80 aa, Selaginella moelendorfii
LNTNGKPRAKRGSATDPQSVYARHRRERINERLKTLQHLVPNGAKVDIVTMLEEAIHYVKFLQLQVNMLSSDEYWTYAPT

SEQ ID NO: 55 - DNA, 240 bp, Selaginella moelendorfii
ctcaacacgaatggcaagccccgagcaaagcgtggatctgcaacagatccccaaagcgtttatgcaaggcaccggagagagaggatcaacgagaggctcaaaactttacaacaccttgttccaaatggtgcaaaggttgacattgtgacaatgcttgaagaagcaatacattacgtgaagtttctacagctgcaagtcaacatgttaagctctgatgagtactggacttatgcacccaca

SEQ ID NO: 56 - protein, 80 aa, Selaginella moelendorfii; identical to SEQ ID NO: 50

SEQ ID NO: 57 - DNA, 240 bp, Selaginella moelendorfii
ctaaacaccgacggaaagccacgcgcaaagcgtggatctgccacggacccgcaaagtatctacgctcggcaaagaagagaaaggatcaacgagcgtttgagagcgctacaaggactcgtaccaaacggagcgaaggttgacattgtgacgatgctcgaggaagccatcaactatgtcaagtttttgcagctgcaagtaaagctgctcagctcggacgagtattggatgtacgcccccaca

SEQ ID NO: 58 - protein, 295 aa, Oryza sativa
MMAAQASSKRGMLLPREAVLYDDEPSMPLEILGYHGNGVGGGGCVDADYYYSWSGSSSSSSSSVLSFDQAAVGGSGGGCARQLAFHPGGDDDDCAMWMDAAAGAMVENTSVVAGGGNNYCHRLQFHGGAAGFGLASPGSSVVDNGLEIHESNVSKPPPPAAKKRACPSGEARAAGKKQCRKGSKPNKAASASSPSPSPSPSPSPNKEQPQSAAAKVRRERISERLKVLQDLVPNGTKVDLVTMLEKAINYVKFLQLQVKVLATDEFWPAQGGKAPELSQVKDALDAILSSQHPNK

SEQ ID NO: 59 - DNA, 888 bp, Oryza sativa
atgatggcagctcaggcaagcagcaagcgcggcatgctgctgccacgggaggcggtgctctacgacgacgagccctccatgccgctggagatcttgggctaccacggcaatggcgtcggcggcggtggctgcgttgacgccgattactactacagctggtcggggtccagctccagctccagctcgtcggtgctcagctttgaccaggcggcggtcggcggcagcggcggcggctgcgcccggcagctggctttccatcccggcggcgacgacgacgactgcgccatgtggatggacgccgccgccggcgccatggtcgagaacacgtctgtcgtcgccggcggcggcaacaactactgtcatcgcctgcagttccacggcggcgccgccggtttcggactcgcgagcccaggctcgtcggtcgttgacaacggcctcgaaatccacgagagcaacgtcagcaagccgccaccgccggcagccaagaagcgcgcatgcccgagcggcgaggcgagagcagcggggaagaagcagtgcaggaaagggagcaagccaaacaaggctgcttctgcttcttctccttctccttctccttctccttctccttctcctaacaaggaacaacctcaaagcgccgctgcaaaggtaagaagagagcggatcagtgagaggctcaaagttcttcaggatctcgtgcctaatggcacaaaggtagacttggtcaccatgctagaaaaggcgatcaactacgtcaaattcctccagctgcaagtgaaggttttggctactgatgagttctggccggcacaaggagggaaagcaccagagctctctcaagtcaaggacgccttggacgccatcctatcttctcagcatccaaacaaatga

SEQ ID NO: 60 - protein, 300 aa, Oryza sativa
MRMALVRERAMVYGGGCDAEAFGGGFESSQMGYGHDALLDIDAAALFGGYEAAASAGCALVQDGAAGWAGAGASSSVLAFDRAAQAEEAECDAWIEAMDQSYGAGGEAAPYRSTTAVAFDAATGCFSLTERATGGGGGAGGRQFGLLFPSTSGGGVSPERAAPAPAPRGSQKRAHAESSQAMSPSKKQCGAGRKAGKAKSAPTTPTKDPQSLAAKNRRERISERLRILQELVPNGTKVDLVTMLEKAISYVKFLQLQVKVLATDEFWPAQGGKAPEISQVKEALDAILSSSSPLMGQLMN

SEQ ID NO: 61 - DNA, 903 bp, Oryza sativa
atgcgcatggcgctggtgcgggagcgcgcgatggtgtacggtggagggtgcgacgccgaggcgttcggcggcgggttcgagtcgtcccagatggggtacggccacgacgcgctgctcgacatcgacgcggcggcgctgttcggggggtacgaggcggccgccagcgccgggtgcgccctcgtgcaggacggcgccgcggggtgggcgggcgcgggcgcgtcgtcctcggtgctggcgttcgaccgcgccgctcaggcggaggaggccgagtgcgacgcgtggatcgaagccatggaccagagctacggcgccggcggcgaggcggcgccgtaccggtcgacgacggccgtcgccttcgacgcggccaccggctgcttcagcctgacggagagagccaccggcggcggcggcggcgcgggtgggcggcagttcgggctgctgttcccgagcacgtcgggcggcggcgtctcccccgaacgcgccgcgccggcgccggcgccccgcggctcgcagaagcgggcccacgcggagtcgtcgcaggccatgagccctagcaagaagcagtgcggcgccggcaggaaggcgggcaaggccaagtcggcgccgaccaccccaaccaaggacccgcaaagcctcgcggccaagaatcggcgcgagaggatcagcgagcggctgcggatcctgcaggagctcgtgcccaacggcaccaaggtcgacctcgtcaccatgctcgagaaggccatcagctacgtcaagttcctccagcttcaagtcaaggttcttgcgacggacgagttctggccggcgcagggagggaaggcgccggagatatcccaggtgaaggaggcgctcgacgccatcttgtcgtcgtcgtcgccgctgatgggacaactcatgaactga

SEQ ID NO: 62 - protein, 294 aa, Oryza sativa
MAMVAGDEAMSVPWHDVGVVVDPEAAGTAPFDAGAGYVPSYGQCQYYYYYDDHHHHPCSTELIHAGDAGSAVAVAYDGVDGWVHAAAAATSPSSSSALTFDGHGAEEHSAVSWMDMDMDAHGAAPPLIGYGPTAATSSPSSCFSSGGSGDSGMVMVTTTTPRSAAASGSQRRARPPPSPLQGSELHEYSKKQRANNKETQSSAAKSRRERISERLRALQELVPSGGKVDMVTMLDRAISYVKFMQMQLRVLETDAFWPASDGATPDISRVKDALDAIILSSSSPSQKASPPRSG

SEQ ID NO: 63 - DNA, 885 bp, Oryza sativa
atggctatggtggccggcgacgaggcgatgtcagtgccatggcacgacgtcggcgtcgtcgtcgaccccgaggcggccgggacggcgccgttcgacgccggcgccggctatgtcccatcgtacggtcagtgccaatactactactactacgacgaccaccaccaccacccgtgcagcacggagctgatccacgcgggcgacgctggcagtgcggttgcggttgcgtacgacggcgtcgacggctgggttcacgccgccgccgcagccacctccccgtcctcgtcatctgcgctcaccttcgatggtcacggcgccgaggagcacagcgcagtgtcgtggatggacatggacatggacgcgcacggcgccgcgcctcccctaatcggctacggcccgacggcggcgacctcctccccctcctcctgcttcagctccggcggctccggcgacagcggcatggtgatggtgaccaccaccaccccgaggagcgccgccgcctctggttcgcagaggcgggcacgcccgccgccgtcgccgttgcagggatcagagctgcacgagtactccaagaagcagcgcgccaacaacaaggagacacagagctcagctgccaagagccggcgggagaggatcagcgagcggctgagggcgctgcaggagctggtgccgagcggcgggaaggtggacatggtgaccatgctggacagggccatcagctacgtcaagttcatgcagatgcagctcagggtgctggagaccgacgcgttctggccggcgtccgacggcgccacgccggacatctcccgggtcaaggacgcgctcgacgccatcatcctctcctcgtcctcgccctcgcaaaaggcttctcctcctcggtcgggctag

SEQ ID NO: 64 - protein, 324 aa, Oryza sativa
MEDSEAMAQLLGVQYFGNDQEQQQPAAAAPPAMYWPAHDAADQYYGSAPYCYMQQQQHYGCYDGGAMVAGGDFFVPEEQLVADPSFMVDLNLEFEDQHGGDAGGAGSSAAAAAAATKMTPACKRKVEDHKDESCTDNVARKKARSTAATVVQKKGNKNAQSKKAQKGACSRSSNQKESNGGGDGGNVQSSSTNYLSDDDSLSLEMTSCSNVSSASKKSSLSSPATGHGGAKARAGRGAATDPQSLYARKRRERINERLKILQNLIPNGTKVDISTMLEEAVHYVKFLQLQIKLLSSDDMWMFAPIAYNGVNVGLDLKISPPQQQ

SEQ ID NO: 65 - DNA, 975 bp, Oryza sativa
atggaggactcggaggcgatggcgcagctgctcggcgtgcagtacttcggcaatgaccaggagcagcagcagccggcggcggcggcgccgccggcgatgtactggccggcgcacgacgcggccgaccagtactacggctcggcgccatactgctacatgcagcagcagcagcattacgggtgctacgacggcggcgcgatggtggccggcggcgacttcttcgtgccggaggagcagctggtggccgacccgagcttcatggtggacctgaacctcgagttcgaggaccagcacggcggcgatgctggcggcgctgggagcagcgccgccgccgccgccgccgccaccaagatgacaccggcgtgcaagaggaaggttgaggatcacaaggatgagagctgcacggacaacgtcgcgaggaagaaggcgcgctccacggcagcaacagtggtgcagaagaagggtaataagaacgcgcagtcaaagaaggcgcagaagggcgcgtgcagccggagcagcaaccagaaggagagcaatggcggcggcgacggcggcaatgtgcagagctcgagcaccaactacctctctgatgacgactcgctgtcgctggagatgacttcgtgcagcaacgtgagctcggcgtccaagaagtcgtcgttgtcatcgccggcgaccgggcacggcggcgcgaaggcgagggccgggcgcggggcggcgaccgatccgcaaagcctctatgccaggaagaggagagaaaggatcaatgaacggctaaagatactgcagaatcttatcccaaatggaaccaaggtggacatcagcacgatgcttgaagaagcagttcactacgtcaagttcttgcagctccaaatcaagcttctgagctcggatgatatgtggatgttcgcgccgatcgcgtacaacggggtcaacgtcgggctcgacctcaagatctctccaccgcagcagcaatga

SEQ ID NO: 66 - protein, 310 aa, Oryza sativa
MESGGVIAEAGWSSLDMSSQAEESEMMAQLLGTCFPSNGEDDHHQELPWSVDTPSAYYLHCNGGSSSAYSSTTSSNSASGSFTLIAPRSEYEGYYVSDSNEAALGISIQEQGAAQFMDAILNRNGDPGFDDLADSSVNLLDSIGASNKRKIQEQGRLDDQTKSRKSAKKAGSKRGKKAAQCEGEDGSIAVTNRQSLSCCTSENDSIGSQESPVAAKSNGKAQSGHRSATDPQSLYARKRRERINERLKILQNLVPNGTKVDISTMLEEAMHYVKFLQLQIKLLSSDEMWMYAPIAYNGMNIGIDLNLSQH

SEQ ID NO: 67 - DNA, 933 bp, Oryza sativa
atggagtccggaggggtgatcgcggaggcggggtggagctcgctcgacatgtcgtcgcaggccgaggagtcggagatgatggcgcagctgcttggaacctgcttcccctccaatggcgaggatgatcatcaccaagagcttccttggtcggttgacacccccagtgcctactacctccattgcaatggaggtagctcaagtgcatacagctctaccactagcagcaacagtgctagtggtagcttcactctcattgcaccaagatctgagtatgaggggtactatgtgagtgactctaatgaggcggccctcgggatcagcatccaggagcaaggtgcagctcagttcatggatgccattctcaaccggaacggcgatccgggcttcgatgatctcgctgactcgagcgttaatctgctggattccatcggcgcttctaacaagagaaagattcaggagcaaggcaggctagatgaccaaacgaaaagtaggaaatctgcgaagaaggctggctcgaagcggggaaagaaggcggcgcaatgtgaaggtgaagatggcagcattgctgtcaccaacaggcaaagcttgagctgctgcacctctgaaaatgattcgattggttctcaagaatctcctgttgctgctaagtcgaatggcaaggctcaatctggccatcggtcagcaaccgatccccagagcctctatgcaaggaaaagaagagagaggatcaatgagaggctcaagattctgcagaaccttgtaccaaatggaaccaaagtagatatcagcactatgcttgaagaggcaatgcattacgtgaagttcttgcagcttcaaatcaagctcctcagctctgatgaaatgtggatgtacgcaccgattgcttacaacgggatgaacatcgggatcgatttgaacctctctcagcattga

SEQ ID NO: 68 - protein, 246 aa, Oryza sativa
MDARCANIWSSADARSEESEMIDQLKSMFWSSTDAEINFYSPDSSVNSCVTTSTMPSSLFLPLMDDEGFGTVQLMHQVITGNKRMFPMDEHFEQQQKKPKKKTRTSRSVSSSSTITDYETSSELVNPSCSSGSSVGEDSIAATDGSVVLKQSDNSRGHKQCSKDTQSLYAKRRRERINERLRILQQLVPNGTKVDISTMLEEAVQYVKFLQLQIKLLSSDDTWMFAPLAYNGMNMDLGHTLAENQE

SEQ ID NO: 69 - DNA, 741 bp, Oryza sativa
atggatgcaaggtgtgcaaacatctggagctctgctgatgcaaggagtgaggaatctgagatgattgatcaactaaagtccatgttctggagcagcactgatgctgaaatcaacttttattctcctgacagtagtgtaaattcttgtgtcacaactagcacaatgcctagcagcttgtttcttcctctgatggatgatgagggatttggcacagtgcaattgatgcatcaggtcatcactgggaacaagaggatgttccccatggatgagcactttgagcagcagcagaagaagccgaagaagaaaacccgaacttctcgctcggtatcaagtagttcaaccattactgactatgagactagctctgaacttgtcaatcctagctgttcctccgggagcagcgtcggagaggattcaattgctgcaactgatggatctgtagtgctgaaacaaagtgacaattcaagaggccataagcagtgctccaaggatacacaaagcctctatgctaagaggagaagggaaaggattaatgagagactgagaatacttcagcagcttgttcccaatggcactaaagttgacatcagcacaatgctggaggaagcagttcagtatgtcaagtttttgcagttgcaaataaagctattgagctctgacgacacatggatgtttgcgcccctagcctataatggcatgaacatggatctcggtcatactcttgctgaaaaccaagaatga

SEQ ID NO: 70 - protein, 198 aa, Oryza sativa
MECSSFEAICNESEMIAHLQSLFWSSSDADPCFGSSSFSLISSEGYDTMTTEFVNSSTNVCFDYQDDSFVSAEETTIGNKRKVQMDTENELMTNRSKEVRTKMSVSKACKHSVSAESSQSYYAKNRRQRINERLRILQELIPNGTKVDISTMLEEAIQYVKFLHLQIKLLSSDEMWMYAPLAFDSGNNRLYQNSLSQE

SEQ ID NO: 71 - DNA, 597 bp, Oryza sativa
atggaatgcagctcctttgaagcaatctgcaatgagtcggagatgattgcgcatttgcagtcattgttctggagcagcagcgatgctgatccttgttttggtagctcatcattttctctcatcagtagtgagggctacgacacaatgaccacagagtttgtgaatagcagcacaaatgtatgttttgattaccaagatgatagcttcgtttcagcagaggagactaccattggtaacaagagaaaagttcagatggatactgagaatgagctgatgacgaaccgcagcaaggaagttcgcaccaagatgtcggtgtcaaaagcatgcaaacattctgtttctgcagagagctcacagtcttattatgcaaagaacaggagacagaggatcaatgagagattgagaatactgcaagaactgatccctaatggaacaaaagttgacatcagcacaatgttggaggaagcaattcagtatgtcaagtttctacacctgcaaatcaagctcttgagctctgatgaaatgtggatgtatgcgccccttgcttttgacagtggtaacaacaggctctatcagaactctctgtcacaagagtag

SEQ ID NO: 72 - protein, 304 aa, Oryza sativa
MEGGGLIADMSWTVFDLPSHSDESEMMAQLFSAFPIHGEEEGHEQLPWFDQSSNPCYYSCNASSTAYSNSNASSIPAPSEYEGYCFSDSNEALGVSSSIAPHDLSMVQVQGATEFLNVIPNHSLDSFGNGELGHEDLDSVSGTNKRKQSAEGEFDGQTRGSKCARKAEPKRAKKAKQTVEKDASVAIPNGSCSISDNDSSSSQEVADAGATSKGKSRAGRGAATDPQSLYARKRRERINERLKTLQNLVPNGTKVDISTMLEEAVHYVKFLQLQIKLLSSDEMWMYAPIAYNGMNIGLDLNIDT

SEQ ID NO: 73 - DNA, 915 bp, Oryza sativa
atggagggtggaggactgatcgccgatatgagctggaccgtcttcgacttgccatcgcacagcgatgagtcggagatgatggcgcagctcttcagtgcattccccatccatggtgaggaggaaggccatgagcagctcccatggtttgatcaatcttccaatccatgctactatagctgcaatgctagcagcactgcatacagcaacagcaatgctagtagcattcctgctccatctgagtatgaaggatactgcttcagtgactcaaatgaggccctgggtgtcagctccagcattgcaccacatgacctgagcatggtccaggtgcaaggtgcaactgagtttctgaatgtgatcccaaaccattcccttgattcattcggtaatggcgagctgggccacgaggatcttgattcggttagtgggactaacaagagaaaacagtcggcagaaggagaatttgatggccaaacaagaggttcaaaatgcgcgagaaaggctgaaccgaagcgagcgaagaaggccaagcaaactgtggagaaggatgcaagtgttgccatcccaaatgggagctgttccatttctgacaatgattccagttcatcccaggaggttgcagatgctggtgctacttcgaaaggcaaatcccgggctggccgcggagcagccactgatccccagagcctctatgcaaggaaaaggagagagaggatcaatgagaggctcaagacacttcagaaccttgtgcccaatggcaccaaagttgatatcagcaccatgcttgaggaggcagtccactatgtgaagttcctgcagcttcagatcaagctcctcagctccgatgaaatgtggatgtatgcgccaattgcgtacaacgggatgaacattgggctcgatctgaacattgatacatga

SEQ ID NO: 74 - protein, 279 aa, Oryza sativa
MAQFLGAHGDHCFTYEQMDESMEAMAAMFLPGLDTDSNSSSGCLNYDVPPQCWPQHGHSSSVTSFPDPAHSYGSFEFPVMDPFPIADLDAHCAIPYLTEDLISPPHGNHPSARVEEATKVVTPVATKRKSSAAMTASKKSKKAGKKDPIGSDEGGNTYIDTQSSSSCTSEEGNLEGNAKPSSKKMGTRANRGAATDPQSLYARKRRERINERLRILQNLVPNGTKVDISTMLEEAVQYVKFLQLQIKLLSSDDTWMYAPIAYNGVNISNIDLNISSLQK

SEQ ID NO: 75 - DNA, 840 bp, Oryza sativa
atggcgcagtttcttggagctcatggtgatcactgcttcacctacgagcaaatggatgagtccatggaggcaatggcagcgatgttcttgcctggccttgacaccgactccaattcttcttctggttgtctcaactacgatgtgcctccacaatgctggcctcagcatggccatagctctagcgtcaccagcttccctgatccagctcatagctatggaagctttgagttcccggtcatggatccgttcccgatcgccgatctcgacgcgcattgcgccatcccctaccttactgaggatctgatcagccctccacatggcaaccatccatcagcaagagtggaagaagctacaaaggttgttacaccagtggctaccaagaggaagtctagtgctgccatgacggcatcaaagaagagcaagaaggctggcaaaaaagatcctattggcagcgacgaaggcggcaacacctacattgatacgcaaagttctagcagttgcacctcagaggaaggaaacctggagggcaacgcgaagccgagctcgaagaagatgggtactagggccaaccgtggggcggcaaccgatccccagagtctctatgcaaggaagaggagagagaggatcaatgaaagattgaggatcctgcagaacttggttcccaatggaacaaaggttgacatcagtacaatgctggaggaagcagtgcagtatgtcaaatttttgcaacttcagattaagttgctaagctctgatgacacgtggatgtatgcaccaatcgcttacaatggagtcaacatcagcaatattgatctgaacatctcttctctgcaaaaataa

SEQ ID NO: 76 - protein, 334 aa, Populus trichocarpa
MALAKDRMGSVQTCPYNGNVMGDFSSMGSYGFDEYQKVAFYEEGNSTFEKTSGLMIKNLAMTSSPSSLGSPSSAISGELVFQATDHQAEEAHSLISFKGIGFDNIMHNNGSLLSFEQSSRVSQTSSQKDDYSAWEGNLSYNYQWNEMNPKCNTSPRLMEDFNCFQRAGNFISMTGKENHGDWLYAESTIVADSIQDSATPDASSFHKRPNMGESMQALKKQCNNATKKPKPKSAAGPAKDLQSIAAKNRRERISERLKVLQDLVPNGSKVDLVTMLEKAISYVKFLQLQVKVLATDELWPVQGGKAPDISQVKEAIDALLSSQTKDGNSSSSPK

SEQ ID NO: 77 - DNA, 1005 bp, Populus trichocarpa
atggcacttgccaaggaccgtatgggatcggttcaaacttgcccctataatggaaatgtgatgggggatttttcctccatggggtcttacggatttgatgaatatcagaaggtagcattttatgaagagggaaatagcacctttgagaaaaccagtgggcttatgatcaagaatttagctatgacctcttctccttcttctcttggcagtccgagcagcgcgatttctggtgaattagtgtttcaggctactgaccatcaagctgaggaagctcattctttgatcagcttcaaaggtatcggattcgataacatcatgcataataatggatctttgcttagctttgagcaaagtagtagggtttctcaaactagtagccagaaagatgactactcagcctgggagggtaatttgagttacaactaccagtggaacgaaatgaatccaaaatgtaacacaagtcctcggttgatggaagattttaattgctttcaaagagctggcaacttcatttccatgactggaaaggaaaatcatggtgattggttatacgctgaatccacaattgttgctgatagcattcaggattctgcaacaccagatgccagcagcttccataagcgtcctaatatgggagagagtatgcaggctctaaagaagcaatgcaacaatgcaacaaaaaagccaaaaccgaagtccgcagcaggtccagctaaggatctacagagtattgctgccaagaatcgacgagagaggattagcgagaggcttaaggtattgcaggatttagtccctaatggctcaaaggttgatttggttactatgctagagaaagccattagttatgttaagtttcttcaattgcaagtaaaggtgttagccactgatgaattatggccagttcaaggtggtaaagctcctgatatttctcaagtaaaggaagccatcgatgccctactctcatctcagactaaagacggaaactcaagctcaagcccaaagtaa

SEQ ID NO: 78 - protein, 330 aa, Populus trichocarpa
MALAKDRMDSVQTCALYGNVMGDLSSLGPNYRFDEEGDRNFEKNSALMIKNLAMSPSPPSLGSPSSANSGELVFQATDNQVEEAHSLINFKGTGFDSIMHANGSLISFEQSNRVSQTSSHKDDYSAWEGNLSCNYQWNQINPKCNANPRLMEDLNCYQSASNFNSITNSAEKENHGDWLYTHESTIVTDSIPDSATPDASSFHKRPNMGESMQALKKQRDSATKKPKPKSAGPAKDPQSIAAKNRRERISERLKMLQDLVPNGSKVDLVTMLEKAISYVKFLQLQVKVLATDEFWPVQGGKAPDISQVKGAIDATLSSQTKDRNSNSSSK

SEQ ID NO: 79 - DNA, 993 bp, Populus trichocarpa
atggcacttgccaaggaccgtatggattcggttcaaacttgcgccctttatggaaatgtgatgggggatctttcctccttggggcctaattatagatttgatgaagagggagataggaactttgagaaaaatagtgcgcttatgatcaagaatttagctatgagcccttctcctccttctcttggcagtccaagcagtgcaaattctggtgaactagtgtttcaggctactgacaatcaagttgaggaagctcattctttgatcaacttcaaaggtaccggatttgatagtatcatgcatgctaatggatctttgattagctttgagcaaagtaatagggtttctcaaactagtagtcacaaagatgactactctgcttgggagggtaatttgagttgcaattaccagtggaaccaaatcaatccaaaatgtaacgcaaatcctcggttgatggaagatcttaattgctatcaaagtgcaagcaacttcaactccataaccaacagtgctgaaaaggaaaaccatggtgattggttatacactcatgaatccacaattgttactgatagcattcccgattctgcaacaccagatgccagcagcttccataagcgtcccaatatgggagagagtatgcaggctctaaagaagcaacgcgacagcgccacaaaaaagccgaaacccaagtctgctggtccagctaaggatccacaaagtattgctgccaagaatcgacgagagcggattagcgagcgccttaagatgttgcaggatttagtccctaacggctccaaggttgatttggttactatgctagagaaagccattagttatgttaagtttcttcaattgcaagtaaaggtgttggccactgatgaattctggccagttcaaggtggtaaagctcctgatatttctcaagtaaagggagccattgatgccacactctcatctcagactaaagacagaaattcaaactcaagctcaaagtga

SEQ ID NO: 80 - protein, 354 aa, Populus trichocarpa
MAEGEWSSLGGMYTSEEADFMAQLLGNCPNQVDSSSNFGVPSSFWPNHEPTTDMEGANECLFYSLDFANINLHHFSQGSSSYSGGSGILFPNTSQDSYYMSDSHPILANNNSSMSMDFCMGDSYLVEGDDCSNQEMSNSNEEPGGNQTVAALPENDFRAKREPEMPASELPLEDKSSNPPQISKKRSRNSGDAQKNKRNASSKKSQKVASTSNNDEGSNAGLNGPASSGCCSEDESNASHELNRGASSSLSSKGTATLNSSGKTRASRGAATDPQSLYARKRRERINERLRILQTLVPNGTKVDISTMLEEAVQYVKFLQLQIKLLSSEDLWMYAPIAYNGMDIGLDHLKVTAP

SEQ ID NO: 81 - DNA, 1065 bp, Populus trichocarpa
atggcagagggagagtggagttctcttggtggaatgtacactagtgaggaggctgatttcatggcacagttgcttggtaactgtcctaatcaggttgattcaagttcaaactttggagttccatctagtttctggcctaaccacgaaccaacaacggacatggaaggggctaatgaatgtttattttattctttggattttgctaatattaatttgcaccatttttcacaagggagtagtagttatagtggtggcagtggcattctttttcccaacacaagccaagatagctactacatgagtgattctcatccaattttggctaacaataatagctcaatgtcaatggatttttgcatgggagactcatatctcgttgaaggcgatgactgctcaaaccaagaaatgagcaatagcaatgaggagcctggtggaaaccagactgtagctgctcttcctgaaaacgattttcgggccaagagagaaccagagatgccagcttctgaactacccctggaagacaaaagcagcaacccacctcagatttctaagaaaagatcacgaaattcaggagatgctcaaaagaacaagaggaatgcaagttcaaagaagagccagaaggttgcctcgactagcaacaatgatgaaggaagtaatgctggccttaatgggcctgcctcaagcggttgctgctcagaggatgaatccaatgcctctcatgagctcaatagaggagcgagttcaagtttgagctcgaaagggactgcaactctcaactcaagtggcaaaacaagagccagcaggggggcagccactgatccccagagtctctatgcaaggaaaagaagagaaagaataaatgagaggctgagaattctacaaacccttgtccccaacggaacaaaggttgacattagcacaatgcttgaagaagctgtccagtatgtgaagtttttgcaactccaaattaagctgctaagctctgaggacttgtggatgtatgcgcctatcgcttacaacgggatggacatcggtcttgatcatctgaaggttaccgcaccatga

SEQ ID NO: 82 - protein, 317 aa, Populus trichocarpa
MEPIGATAEGEWSSLSGMYTSEEADFMEQLLVNCPPNQVDSSSSFGVPSSFWPNHESTMNMEGANECLLYSLDIADTNLYHFSQVSSGYSGELSNGNVEESGGNQTVAALPEPESNLQPKRESKMPASELPLEDKSRKPPENSKKRSRRTGDAQKNKRNVRSKKSQKVASTGNNDEESNGGLNGPVSSGCCSEDESNASQELNGGASSSLSSKGTTTLNSSGKTRASKGAATDPQSLYARKRRERINERLRILQNLVPNGTKVDISTMLEEAVQYVKFLQLQIKLLSSEDLWMYAPIAYNGMDIGLDHLKLTTPRRL

SEQ ID NO: 83 - DNA, 954 bp, Populus trichocarpa
atggagcctattggagccactgcggagggagagtggagttctcttagtggaatgtacacaagtgaggaggctgatttcatggaacagttgcttgtcaactgtcctcctaatcaggttgattcaagttcaagctttggagttccatctagtttttggcctaaccatgaatcaacaatgaacatggaaggggccaatgaatgtttattgtattctttggatattgctgatactaatctgtaccatttttcacaagtgagcagtggttatagtggtgaattgagcaatggaaatgtggaagagtctggtggaaaccagactgtagctgctcttcctgaacctgaaagcaatttgcaacccaagagagaatcaaagatgccagcatctgaactacccctggaagataaaagcagaaagccacctgagaattccaagaaaagatcacgacgtacgggagatgcccaaaagaacaagaggaatgtaaggtcaaagaagagccagaaggttgcctcgactggcaacaatgatgaagaaagcaatggtggccttaatggtcctgtctcaagcggttgctgctcagaggatgaatccaatgcctcccaggagctcaatggaggagcgagttcaagtttgagctcaaaagggacaacaactctcaactcaagtggcaaaacaagagccagtaagggggcagccactgatccccagagcctctatgcaaggaaaagaagagaaagaataaatgagaggctgagaattctacaaaaccttgtccccaatggaacaaaggttgacattagcacaatgcttgaagaggctgtccagtatgtgaagtttttgcaactccaaattaagctgctaagctctgaagacctgtggatgtatgctcctatcgcgtacaatggtatggacatcggtcttgatcatctgaagcttaccacaccaagacgattgtag

SEQ ID NO: 84 - protein, 362 aa, Populus trichocarpa
MNTQAMEAFRDGELWNFSRMFSMEEPDCTPELLGQCSFLQDTDEGLHFTIPSAFFPAPESDASMAEDESLFYSWHTPNPNLHFDSQESSNNSNSSSSVFLPYSSHESYFFNDSNPIQATNNNSMSMDIMDEENIGLFMPLFPEIAMAETACMNGDMSGDKTGDLDDNLKPAANDVLAKGLQLKRKLDVPEPIANTLDDMKKKARVTRNVQKTRKVGQSKKNQKNAPDISHDEEESNAGPDGQSSSSCSSEEDNASQDSDSKVSGVLNSNGKTRATRGAATDPQSLYARKRRERINERLKILQNLVPNGTKVDISTMLEEAVHYVNFLQLQIKLLSSDDLWMYAPLAYNGIDIGLNQKLSMFL

SEQ ID NO: 85 - DNA, 1089 bp, Populus trichocarpa
atgaatacgcaggctatggaagcctttcgtgatggagaattatggaacttcagcagaatgttctccatggaagagcctgattgcaccccagaattacttggtcagtgctcttttcttcaggatactgatgaaggattgcattttacaatcccatcagctttcttccctgctcctgaatccgacgcgagcatggctgaggacgagagtttgttttattcttggcatactcccaaccccaatttgcattttgattctcaagaaagtagtaataacagtaattctagcagtagtgtatttcttccctattccagccatgaatcctacttcttcaatgattctaatcccattcaagctacgaacaataactctatgtccatggatattatggatgaggaaaatattggcttgtttatgccactttttcctgaaattgcaatggcagaaactgcctgtatgaatggagatatgagcggtgacaaaacaggagatttagatgataatctgaagccagcagctaatgatgttctggccaagggattgcagctcaaaaggaagcttgatgttccagaaccaatagccaacacattggacgacatgaagaaaaaagcccgggttacaagaaatgtgcaaaagactaggaaggttggacagtcaaaaaaaaatcagaagaacgcaccagatattagccatgatgaagaagagagtaatgctggaccagacggacaaagttccagcagttgtagttcagaagaggacaatgcctctcaggattctgattccaaggtttctggagttctcaattccaatggaaaaacaagagctactaggggagctgccacagacccccagagcctttatgcaaggaaaagaagggagaggataaacgagagactgaaaatcttgcagaatcttgtccctaacggaaccaaggttgatatcagcacgatgctagaagaggcagtccattacgtaaactttttgcagcttcaaatcaagcttttgagctcggatgatctatggatgtatgcacctctggcttacaatggaatagatattggactcaaccagaagctctctatgtttctatga

SEQ ID NO: 86 - protein, 302 aa, Musa acuminata
MAQESTWSSFDATMLAEEESRMIAQLLSNYQCFGEQDRDVGCCELPPSSCCSSHAADSCYCWSANENSNPGLCYWSQSGDESDGAHAIGTVPVFTNHCLVGDQVAVNQTLSIHEPTAAHAEMPKRKIESHASEDDFRRQSSKKKLQAPTNALKSVKKARPGRNQKSIVCGDEEENNARSSGRSCCSYSSEEDSQAFQADLNAKTRSNRWPATDPQSLYAKQRRERINARLRTLQNLVPNGTKVDISTMLEEAVRYVKFLQLQIKLLSSDELWMYAPVVHSGMIDGQVNSEIFVSANTRNEWF

SEQ ID NO: 87 - DNA, 909 bp, Musa acuminata
atggctcaggagtcaacttggagctcgtttgatgctacaatgcttgctgaggaggagtcccgaatgatcgcacaattgctcagcaactaccagtgttttggcgagcaagatcgagatgttggatgctgtgaactcccgccatcgtcttgttgttcttctcatgcagctgattcatgttactgttggtcagcaaatgagaacagtaacccgggtttgtgctactggtctcagagtggagatgaatccgatggagcacatgcaatcggcactgtgccggtcttcacgaaccattgcttggtgggagatcaagtcgctgtgaatcaaactttgagcattcacgaacctactgctgctcatgcagagatgccaaagcgcaagatagagtctcatgcttctgaagatgatttccgtcgtcaaagttctaagaaaaagcttcaggctccgacgaatgctctgaagagcgtgaagaaggcacgacctgggaggaaccagaagagcattgtgtgtggtgatgaggaagagaacaatgccaggagcagtggccggagttgctgcagctacagctctgaggaagactcacaagctttccaggctgatcttaatgcaaaaacacgatcgaatcgatggccagccacagatcctcaaagcctctatgcaaagcaaagaagggaaagaatcaatgctagattgaggacattgcagaacctggtgcctaatggaactaaagttgacattagcacaatgctcgaagaagctgttcgttacgtcaagttcttgcagctgcagataaagcttttgagctcggatgagctgtggatgtacgctcctgttgtccacagtgggatgattgatggccaagtcaactcagagatatttgtgtctgcaaatactcgtaatgagtggttctga

SEQ ID NO: 88 - protein, 366 aa, Medicago truncatula
MEPIGTFPEGEWDFFRKMFASEDHEYYSQQFLDQNSLLLGENDGLNNGTQSTFCTAEIGENERMFYSFDHAHIQNSNYIPQTQENSYNSNSSASDDTNYYFSYPNHVLENNINNCISNDFRMDENLFASSVPSLNEIVMEENVRMNEDSASDDHIVEKNGYNTQIMEPFDLHTKHEMQMKLKRKLDVIEVEVPVEEKINNNPKKKPRVSNDGQGCMKNARSKKNHKVIASHEEEMTEEINRGSNGNSSSSNISEDDNASQENSGGTTLNSNGKTRASRGSATDPQSLYARKRRERINERLRVLQNLVPNGTKVDISTMLEEAVNYVKFLQTQIKLLSSDDMWMYAPLAYNGLDLGLNLNLNSSLPL

SEQ ID NO: 89 - DNA, 1101 bp, Medicago truncatula
atggaacctataggtactttccctgaaggagaatgggatttctttcgcaaaatgtttgcaagtgaagatcatgaatattactcacaacaatttcttgatcaaaattcacttcttctaggggaaaatgatgggttgaacaatggaacacagtccacattttgcactgctgaaattggtgaaaatgagcgtatgttttattcttttgatcatgctcatatccaaaactctaactatattcctcaaactcaagagaatagttacaatagcaattctagtgctagtgatgatacaaattactattttagttatcctaatcatgtactagaaaataatattaataattgtatatccaatgattttcgcatggatgagaatttgtttgcttcttctgttccatcccttaatgagattgtaatggaagagaatgtgagaatgaatgaagattctgcaagtgatgatcatattgtggagaaaaatggttacaatactcaaataatggaaccttttgatcttcacaccaagcatgagatgcaaatgaagctcaaaaggaaacttgatgtgatagaagtggaggttcccgttgaagaaaaaattaacaacaatccgaagaaaaaacctcgtgtttcgaatgatggccaaggatgcatgaaaaatgcaaggtcaaagaagaaccacaaagttattgctagccatgaagaggagatgacagaagagattaatagaggatcaaatggaaatagttctagtagtaacatttctgaggatgataatgcttctcaagaaaatagtggaggaactactctcaactcaaatgggaagacaagagctagtagaggatctgcaacagatccccaaagtctatatgcaaggaaaagaagagagagaataaatgaacgactaagagtcttacaaaatcttgtaccaaacggaacaaaggttgatatcagtacaatgcttgaagaggcagtcaattatgtgaaatttttacagactcaaatcaagcttttgagctctgatgatatgtggatgtatgcaccacttgcttacaatggacttgaccttggactcaatctcaacctcaacagctctctaccactatga

SEQ ID NO: 90 - protein, 258 aa, Glycine max
  UNSURE (1)..(1): Xaa is unknown
XFLCFSQGSSSSTDNSGNNIFSITSSGAYSCDPEANFDSVSMVLCLGDAKFSPHSFQCDDNSNQQINENTDEESSLDPWKLAIADNNLQAKREYEMMVSEPVEVDRSRNLENLAKRLKSSIEVSKTLRSAKSGKNSKSASVSNDEDDRSLSLQAQRNSCFSQSDSNAYLEPNGGASKDPAPPNLHRKSRATTGAATDPQSLYARKRRERINERLRILQNLVPNGTKVDISTMLEEAVQYVKFLQLQIKLLSSDDLWMY

SEQ ID NO: 91 - DNA, 773 bp, Glycine max
attttttgtgtttctcacaagggagtagctccagtactgataatagtggtaataatatcttttccattacaagtagtggagcctactcctgtgatccagaagcaaactttgattctgtgtccatggttttgtgccttggagatgccaaatttagtccccatagttttcaatgtgatgacaactcaaaccaacagataaatgaaaacactgatgaagagtcaagtctagacccatggaagttggctatagctgacaataatttgcaggctaagagggagtatgaaatgatggtttctgaacctgtagaagtggatagaagcagaaacctggagaacctagcaaaaagactaaagagttcaatagaggtttcaaaaacattgaggagtgctaaatcagggaaaaattcaaaatctgcttcagtgagcaacgatgaagatgatagaagcttgagcctccaagcccaaaggaatagctgtttttcacagagtgactctaatgcttatctggagccaaatggaggggcatcaaaagatcctgcacctcccaatttgcatagaaaatcaagagcaactaccggtgctgccactgatccacagagcctctatgcaagaaagagaagagaaagaataaatgaaaggttgagaatactgcaaaatcttgttcccaacggaactaaggtggatatcagcaccatgcttgaggaagctgtccaatacgtgaagtttttacagctccaaattaagcttctgagctctgacgatctgtggatgtat

SEQ ID NO: 92 - protein, 176 aa, Glycine max
  UNSURE (1)..(1): Xaa is unknown
XNLENLPKRLKSSIEVPKTSRNAKSRKNSKSASTSNDEDDRSLSLQVQRNNSCFSQSDSNAYLEPNGGASKDPAPPNLDRKSRATTSAAADPQSLYARKRRERINERLRILQNLVPNGTKVDISTMLEEAVQYVKFLQLQIKLLSSEDLWMYAPIVYNGINIGLDLGISPTKGRSM

SEQ ID NO: 93 - DNA, 571 bp, Glycine max
gaaacctggagaacctaccaaaaagactaaagagctcaatagaggtcccaaaaacatcgaggaatgctaaatcaaggaaaaattcaaaatctgcttcaactagcaacgatgaagatgatagaagcttgagcctccaagtccaaaggaataatagctgtttttcacagagtgactctaatgcttatcttgagccaaatggaggggcatcaaaagatcctgcacctcctaatttggatagaaaatcaagagcaactaccagtgccgccgctgatccacagagcctctatgcaagaaagagaagagaaagaataaatgaaaggctgagaatactgcaaaatcttgtccccaacggaactaaggtggatatcagcaccatgcttgaagaagctgtccaatacgttaagtttttacagctccaaattaagcttctgagctctgaagatttgtggatgtatgctccaattgtttacaatggaataaacattggactagacctcggtatttctccaaccaaaggaagatcaatgtgatagcatagcaattaaagaggatataatatttcattaactta

SEQ ID NO: 94 - protein, 108 aa, Lactuca saligna
  UNSURE (1)..(1): Xaa is unknown
XRSKEAEILSSNGKRKASRGSATDPQSVYARKRRERINERLRILQNLVPNGTKVDISTMLEEAVEYVKFLQLQIKLLSSDDMWMYAPIAYDGMDIGLHSTTIPSSSTR

SEQ ID NO: 95 - DNA, 419 bp, Lactuca saligna
tgagatcaaaagaggctgaaattctgagctcaaatggcaagagaaaagcaagtagggggtcagcaactgatccacaaagtgtctatgcacggaaaagaagagaaagaattaacgaacgtttaagaatattacaaaatcttgttcctaatggtacaaaggttgatataagcacaatgcttgaagaggctgttgagtacgtgaagtttttgcagcttcaaatcaagctcttgagctccgatgatatgtggatgtatgctccgattgcatacgatggaatggacattgggcttcattcaacaaccatcccatcatcgtcaacaagataatgcaaagttgggctatccatattgtcacatttttgttgaataaaaggcaatcgataacaaaattcaaagtttataaagagtacacatttatgc

SEQ ID NO: 96 - protein, 274 aa, Triticum aestivum
MASKRATTRELRAMYDDEPSSMSLELFGYHGVVVDGDDENDDTATALPQLSFVDNFKGGCGSAADYYSWAYNASGGTPGASSSSTSSVLSFEHAGGAGHQLAYNSGTGDDDCALWMDSMADHQHGAARFGFMNPGSADVVPEIQESSIKQPAKSAQKRSSSGGEAQAAAKKQCGGGRKSKAKVVPTKDPQSAVAKVRRERISERLKVLQDLVPNGTKVDMVTMLEKAITYVKFLQLQVKVLATDEFWPVQGGKAPELSQVKTALDAILSSQQQP

SEQ ID NO: 97 - DNA, 825 bp, Triticum aestivum
atggcgagcaagcgggccaccacgcgggagctccgggcgatgtacgacgacgagccctcctccatgtccctcgagctcttcggctaccatggcgtggtcgtcgacggtgacgatgaaaacgacgacactgccaccgccctgccccagctctccttcgtcgacaacttcaaaggtgggtgcgggtcggcggcggactactacagctgggcgtacaacgcctccggcgggacgccgggcgcctcctccagctccacctcgtcggtgctcagctttgagcatgccggcggtgccggtcatcagctggcttataattccggcacaggcgacgatgactgcgcgctctggatggacagcatggccgatcatcagcacggcgcggccaggtttgggttcatgaacccagggtcggccgatgtcgtcccagaaatccaggagagcagcatcaagcagccggccaagtctgcgcagaagcgctcgagctcgggtggtgaggcgcaagcagcggcgaagaagcagtgtggaggaggcaggaagagcaaggccaaagttgtccctaccaaggatcctcagagcgctgttgcaaaggtccgaagagagcgcatcagtgagaggctcaaagttctgcaggatcttgtacccaacggcacgaaggtggacatggtcaccatgctcgagaaggcaatcacctatgtcaagttcctgcagctgcaagtcaaggtgttggcgaccgacgagttctggccggtgcaaggagggaaggcgccggagctctcccaagtgaagaccgcgctggacgccatcctttcttcccagcagcaaccctag

SEQ ID NO: 98 - protein, 18 aa, Carthamus tinctorius
DSQIIHPMPCDELHKSLI

SEQ ID NO: 99 - DNA, 769 bp, Carthamus tinctorius
gattcacagataatccaccctatgccgtgtgatgaactccacaaatccttaatttaattgtaccacatcaggcgacgttatccatattgggtgttcactgatggtgaaagcacatctttcgcgcgacctctactcaatgactcaagaattagaggtgaactattgcttacactatctactactaaacattgtaaagtgactgccagttctatgagacgttcgtatagcatgatgcatgatcatgagaaaagctaaaagatacagcgcagaaagagccagaagctcgtttctaaaggcaacgaaagtgaagctgaccatgatgcagtttttgggcaaataatgaaaatgtgtggatctgacaatgactcgaattggcctcgggagtcgagcacaagtccaagaccaaaagaggctgcaaatctgaactcaaatgggaagacaaaagcaaatagggggtcagcaacggatccacaaagtgtctacgcacggaagagaagagaacgaattaatgaacggttaagaatactacagagtctggttcctaatggtacaaaggttgatataagcacaatgcttgaagatgctgtccagtatgtgaaatttttgcagctccaaatcaagccgttgagctctgatgatctgtggatgtatgcccccatcgcgtacaacgggatggagacggggcttgattctacgatcccctcgccaaggtgaagactatccaaagttgccgcatcttttttcttgaaaaaagggaagcctggggcaa

SEQ ID NO: 100 - protein, 309 aa, Brachypodium distachyon
MALVREPMVLYDGGFDASEASAFDSIGCFGHGHGHDALLGGVDAAALFGGYAHDEPAGASASAYVKDGSHWAGVGASVLAFDRAARGHGAQAMATAAAQEEEECDAWIDAMDEDNGEAAPAPSIGFDPATGCFSLTQRPGAGARRPFGLLFPSASGGAPSPDSAAPAPASRGSQKRPSAGIARAQDAEPRASKKQCGASRKTTAKAKSPAPAITSPKDPQSLAAKNRREKISERLRTLQEMVPNGTKVDMVTMLEKAISYVKFLQLQVKVLATDEFWPAQGGMAPEISQVKEALDAILSSQRGQFNCSS

SEQ ID NO: 101 - DNA, 930 bp, Brachypodium distachyon
atggcattagtgcgggagccgatggtactgtatgacggcggtttcgacgcctcggaggcgtcggcattcgactccatcggctgcttcggccacggccacggccacgacgcgctcctaggcggcgtcgacgcggccgcgctgttcgggggctacgcgcacgacgagccggccggcgccagcgccagcgcctacgtgaaggacggctcgcactgggccggcgtgggtgcgtccgtgctcgcgttcgaccgtgccgctcggggccacggcgcgcaggccatggcgaccgcggccgctcaggaggaggaagaatgcgacgcgtggatcgacgccatggacgaggacaatggcgaggcggcgccggcgccgtccatcggcttcgacccggccacgggctgcttcagcctcacgcagcggcccggcgccggcgcgcggcgcccgttcgggctcctgttcccgagcgcgtccggtggcgcgccctcgcccgacagcgccgcgccagcgccggcatcccgcggttcccagaagcggccatccgccgggattgcgcgcgcgcaggacgcggagccgcgggccagcaagaagcagtgcggcgcgagcaggaagacgacggccaaggcgaagtcgcctgcgcctgccatcacctcgcccaaggacccgcagagcctcgctgcaaagaaccggagggagaagatcagcgagcggctccggacgttgcaggagatggtgcccaacggcaccaaggtggacatggtcaccatgctcgagaaggccatcagctacgtcaagttcctgcagctgcaagtcaaggtgctcgcgacggacgagttctggccggcgcagggagggatggcgccggagatctcccaggtgaaggaggcgctcgacgccatcctgtcgtcgcagagggggcaattcaactgctccagctag

SEQ ID NO: 102 - protein, 272 aa, Brachypodium distachyon
MASRHATTREPHLRTMYDDEPSMSLELFGYHGVVVDGDDDGDTATDLPQLTFVDNFKGGCGSADYYGWAYSASGGASGACSSSSSSVLSFEQAGGAGHQLAYNAGTGDDDCALWMDGMADQHDTAKFGFMDPGMSDVSLEIQESSMKPPAKMAQKRACQGGETQAAAKKQCGGSKKSKAKAAPAKDPQSAVAKVRRERISERLKVLQDLVPNGTKVDMVTMLEKAITYVKFLQLQVKVLATDDFWPVQGGKAPELSQVKDALDAILSSQNQS

SEQ ID NO: 103 - DNA, 819 bp, Brachypodium distachyon
atggcaagcaggcacgccactacacgggagccacacctccggaccatgtacgacgacgagccatccatgtccctcgagctcttcggctaccatggcgtcgtcgtcgacggtgacgacgatggcgacaccgccaccgaccttccccagctcacctttgttgacaacttcaaaggcgggtgtgggtcagccgactactacggctgggcgtacagcgcctccggtggtgcgtcaggcgcctgctccagctccagctcgtcggtgctcagctttgagcaggcgggtggtgccggtcatcagctggcttataacgccggcacaggtgacgatgactgcgcgctctggatggacggcatggctgaccagcatgacacagccaagtttgggttcatggacccaggcatgtctgatgtcagcctagaaatccaggagagcagcatgaaaccgccggccaagatggcacagaagcgcgcttgccagggtggtgagacgcaagcagcggcgaagaagcagtgtggaggaagcaagaagagcaaggcaaaagctgcccctgccaaggatcctcaaagcgccgttgcaaaggtccgaagagagcgcatcagcgagaggctcaaagttctgcaggatctcgtgcccaatggcacaaaggttgacatggtcaccatgctcgaaaaggcaatcacctatgtcaagttcctgcagctgcaagtcaaggtattggcgactgatgacttctggccggtgcaaggagggaaagctccggagctctcccaagtgaaggacgctctggacgcgatcctgtcttcccagaatcaatcctag

SEQ ID NO: 104 - protein, 240 aa, Brachypodium distachyon
MALVGQATKLCYDGFAGDGVPPFMDAACLAFDHGYDYNNPHAWEFPTGAEPGNSSAFDVAWTGVSSTSPVLTFDAAEWMDATATDRLSSYSPSAATVPASYKRPRAHVQPQQEAEEQESITPNPKKQCGDGKVVIKSSAAATGTSPRKEPQSQAAKSRRERIGERLRALQELVPNGSKVDMVTMLDKAITYVKFMQLQLTVLETDAFWPAQGGAAPEISQVKAALDAIILSSSQKPRQWS

SEQ ID NO: 105 - DNA, 723 bp, Brachypodium distachyon
atggctctagtgggtcaggcaacgaagctctgctacgacggcttcgccggagacggtgtgccgccgttcatggacgcagcttgtctggcattcgaccacgggtatgattacaacaatccccacgcatgggaattccccaccggcgccgagccaggcaacagcagcgcgttcgacgttgcctggaccggcgtctcctccacttctccggtgctcacattcgacgccgccgagtggatggacgccacggccacggaccggctgagctcctacagcccgtctgcggccaccgtgccggcctcttacaagcggcctcgtgcgcacgtgcagccacagcaggaagcagaagaacaggaaagcattactcccaatcccaagaagcagtgcggcgatgggaaagtagttatcaagtcatcggcggcggctaccggcaccagtccacgcaaggaaccccaaagccaagctgccaagagccgtcgtgagcggatcggcgagcggctgagagcgctgcaggagctggtgcccaacggcagcaaggtggacatggtcaccatgctcgacaaggccatcacttatgtcaagttcatgcagctccagctcacggtgctcgagacagacgcgttctggcctgcgcagggtggcgcggcgccggagatctcccaggtgaaggcggcgctcgacgccatcatcctctcctcgtcgcagaagcctcgtcagtggagctag

SEQ ID NO: 106 - protein, 821 aa, Brachypodium distachyon
MEAGGLISEAGWTMFDFPSQGEESEIMSQLLGAFPSHLEEGHQDLPWYQASDPSYYDCNLNTSSESNASSLAVPSECMGYYLGDSSESLDLSSCIAPNDLNLVQEQDATEFLNMTPNLSLDLRGNGESSCEDLTSVGPTNKRKHSSAEEGIDCQARGQKFARKAEPKRTKKTKQSGWEVAVATRNGSTASCCTSDDDSNASQESADTGVCPKGKARAARGASTDPQSLYARKRRERINERLKTLQTLVPNGTKVDMSTMLEEAVHYVKFLQLQIKVLSSDDMWMYAPLAYNGMNIGLDLNIYTPERWRTASAAPSTEGREYAGVDRISDLPDGILGDIVSLLPTAEGARTQILKRRWRHIWRCSAPLNLDCCTLVARGGGREAEDELVGLIPSILSSHQGTGRRFHVPSSRHSDRAATIEAWLQSAALDNLQELDLWCTHTYLYDYVPLPPAVFRFSATVRVVTIANCNLRDSAVQGLQFPQLKQLGFKDIIIMEDSLHHMIAACPDLECLMIERSLGFACVRINSLSLRSIGVSTDHPHPHELQFVELVIDNAPCLKRLLHLEMCYHLDMHITVISAPKLETLSCCSSVSRSSTKLSFGSAAIQGLHIDSLTTVVRTVQILAVEMHSLCLDTIIDFMKCFPCLQKLYIKSFVSGNNWWQRKHRNVIKSLDIRLKTIALESYGGNQSDINFVTFFVLNARVLELMTFDVCSEHYTVEFLAEQYRKLQLDKRASRAARFHFTSNRCVRGIPYIGRAELFLPIKCSHVDTSPNLSSFRLSAVFSVCITRNLLRLKKAMWVISLYYSPEFTKQVAVHNPNEMPF

SEQ ID NO: 107 - DNA, 2466 bp, Brachypodium distachyon
atggaggctggagggctgatttctgaggctggctggaccatgtttgacttcccgtcgcaaggcgaggaatcagagatcatgtcgcagctgctaggcgccttcccctcccatcttgaggaaggccatcaggatctgccttggtaccaggcttctgacccatcctactatgactgtaatcttaatacaagtagtgaaagcaatgctagtagtcttgctgttccatccgagtgtatgggctactatttgggtgattcaagtgagtccctggacctgagctcctgcattgcaccaaatgacctgaacttggtccaggagcaagatgcaactgagtttctgaatatgacaccaaatctttcccttgatttacgtgggaatggtgagtcgagctgcgaggatctcacttcggtcggtcctactaacaagcgaaagcactcctcggcagaagaaggaatcgactgccaagcaagaggccagaaattcgccagaaaggctgaaccgaagcgaacaaagaagaccaagcaaagcggatgggaggttgctgttgccaccaggaatggaagcacagcgagctgctgcacctctgatgatgactcaaacgcttctcaagaatctgcagataccggtgtttgtccgaaaggcaaggctcgggctgcccgtggcgcatcaactgatccccagagcctctatgcaaggaaaaggagggaaaggatcaatgagagactgaagacactgcagacccttgtgcccaatggaaccaaagtagatatgagcaccatgcttgaggaggcagtccactacgtgaagttcctgcagcttcagatcaaggtcttgagctctgatgatatgtggatgtatgcgccgctagcatacaacgggatgaacattgggcttgatctgaacatatatactccggagaggtggaggacagcgtccgcggcgccctcaaccgaagggcgtgaatacgccggcgtcgaccgcatcagcgacctccccgacggcatcctcggcgacatcgtctcgttgctccccaccgccgaaggagcccgcacccagatcctcaagcgcaggtggcgccacatctggcgctgctccgcccctctcaacctcgattgctgtaccttggtcgcccgtggcggcggccgtgaggctgaagatgaactcgtcggtctcataccgtccatcctttcttctcaccaaggcaccggccgccgcttccacgtcccctcgtcgcgccactctgaccgagctgctaccattgaagcctggctccaatctgctgccctcgacaatctccaggagctcgatttatggtgcacccacacctatctttacgactatgttccgctgccacccgccgtctttcgcttctccgccaccgtccgtgttgtcaccatcgcaaattgtaacctccgtgacagcgccgtccaaggccttcaattcccacaacttaaacagctcggattcaaagatatcatcatcatggaggattcgctgcaccacatgattgctgcgtgtccagatctcgagtgcttgatgattgaaaggagcttaggttttgcttgcgtccggatcaattcccttagtcttagaagcatcggtgtgagcactgaccaccctcacccacatgagctccagtttgtggaactcgtcattgataatgcaccttgtcttaagagattgctccatcttgaaatgtgttatcaccttgacatgcatataacagtaatctccgcgcctaaactggagaccttgagctgctgttcttctgtgagtcgctcctccaccaaactctcgtttggctccgcggccattcagggattgcacattgatagcctaacaacagtggtgcgcactgtccaaattttagctgtagagatgcattctctttgtctagacacaattattgacttcatgaaatgctttccatgtctgcagaagttgtacattaagtcatttgtaagtggaaacaattggtggcaacgtaaacaccggaacgttatcaaatcccttgacatccgtctcaagacaatagcgttggaaagttatgggggcaatcagtctgacatcaactttgtcacattctttgtcttgaacgcgagagtgctagagttgatgacatttgacgtttgttctgagcattacactgtggagttcttggcagagcaatataggaagcttcagctagataagagggcttcaagagccgctcggttccattttacaagtaaccgatgtgtccgtggtattccgtatatcggacgtgccgagctattcttgcctatcaaatgttctcatgttgacaccagtccaaacttgagtagtttccgtttgtctgcagtattttcagtttgtattacccggaaccttttgcgtttaaaaaaagctatgtgggtcattagtttgtattattctccagaatttacaaaacaagtggccgtgcacaatcccaatgaaatgccgttttag

SEQ ID NO: 108 - protein, 256 aa, Brachypodium distachyon
MEAKCGAIWSSIDARSEDSEMIAHLQSMFWSNSDVALNLCSSNTSGNSCVTASTLPSSLFLPLVDNESYGAAPSVDTGMDSCFDHQHQSITGHKRISHMDEQMKKTRKKSRTVPSVSKALGSSLVDNQMNADIFNQSSSCCSSGEDSIGTSEKSIVANQSDNTSGCKRPSKNMQSLYAKKRRERINEKLRVLQQLIPNGTKVDISTMLEEAVQYVKFLQLQIKVLSSDETWMYAPLAYNGMDIGLTLALRTAANQE

SEQ ID NO: 109 - DNA, 771 bp, Brachypodium distachyon
atggaggccaagtgtggagctatttggagctctatcgatgcgaggagcgaggactctgagatgattgctcacctgcagtccatgttctggagcaacagtgatgttgctctcaacctctgttcgtcaaacaccagtggcaattcttgtgtcacagctagcacattgcctagcagcttgttccttcctcttgtcgataatgagagctatggtgcagcgccatcggtggacaccggcatggattcatgctttgatcaccagcatcagagcattactggtcacaagaggatatcgcacatggatgagcagatgaagaagacgagaaagaagtcccggactgttccatcggtatcaaaggctctgggttccagcctagtcgataatcagatgaatgctgacattttcaatcagagctcctcctgctgcagctcgggagaagattcaattggaacatctgagaaatccattgttgcaaaccagagtgacaatacgagtggttgtaagcggccttcaaagaatatgcaaagcctttatgcaaagaagagaagagagaggatcaacgagaagttgagagtactgcagcagctgattcccaatggcaccaaagttgacatcagcacaatgttggaggaagcagttcagtatgtcaagtttctgcagctgcaaataaaggtcttaagctctgacgagacatggatgtatgcgcccctcgcctacaatggtatggacatcggtctcactctcgctctgagaactgctgcaaaccaagagtga

SEQ ID NO: 110 - protein, 283 aa, Zea mays
MALVREHGGYYGGFDSVEAAAFDTLGYGHGASLGFDASSALFGEGGYAAGGGDAWAGAGASTVLAFNRTTAAAAVGVEEEEEECDAWIDAMDEDDQSSGPAAAAPEARHALTASVGFDASTGCFTLTERASSSSGGAGRPFGLLFPSTSSSGGTPERTAPVRVPQKRTYQAVSPNKKHCGAGRKASKAKLASTAPTKDPQSLAAKQNRRERISERLRALQELVPNGTKVDLVTMLEKAISYVKFLQLQVKVLATDEFWPAQGGKAPEISQVREALDAILSSAS

SEQ ID NO: 111 - DNA, 849 bp, Zea mays
atggcgttggtgagggagcacggtgggtactacggaggcttcgacagcgtcgaggcggcggccttcgacacgctcggctacggccacggcgcgtcgctgggctttgacgcgtcgtcggcgctgttcggggaaggcggttatgcggcgggcggcggggacgcctgggcgggcgcgggggcgtcgaccgtcctggcgttcaaccgcacaacggcagcggcggccgtgggtgtggaagaggaggaggaggagtgcgacgcgtggatcgacgctatggacgaggacgaccagagctccggccccgccgcggcggcgccagaggcgcgccacgcgctgacggcctccgtgggtttcgacgcctccacggggtgcttcaccctgacggagagggcgtcgtcgtcgtcaggcggagcggggcgcccgttcggcctgctgttcccgagcacgtcgtcgtcgggcggcacgcccgagcgcacggcgccggtgcgcgtcccgcagaaacggacctaccaggctgtgagccccaacaagaagcactgcggcgcgggcaggaaggcgagcaaggccaagctcgcgtccacagccccaaccaaagatccccagagcctcgcggccaagcagaaccggcgcgagcggatcagcgagcggctgcgggcgctgcaggagctggtgcccaacggcaccaaggtcgacctggtcaccatgctcgagaaggccatcagctacgttaagttcctccagttgcaagtcaaggttctggcaacagacgaattctggccggcacagggagggaaggcgccggagatctcccaggtgagggaggcgctcgacgccatcttgtcgtcggcgtcg

SEQ ID NO: 112 - protein, 279 aa, Zea mays
MAQFLGAADDHCFTYEYEHVDESMEAIAALFLPTLDTDSANFSSSCFNYAVPPQCWPQPDHSSSVTSLLDPAENFEFPVRDPLPPSGFDPHCAVAYLTEDSSPLHGKRSSVIEEEAANAAPAAKKRKAGAAMQGSKKSRKASKKDNIGDADDDGGYACVDTQSSSSCTSEDGNFEGNTNSSSKKTCARASRGAATEPQSLYARKRRERINERLRILQNLVPNGTKVDISTMLEEAAQYVKFLQLQIKLLSCDDTWMYAPIAYNGINIGNVDLNIYSLQK

SEQ ID NO: 113 - DNA, 840 bp, Zea mays
atggctcagtttcttggggcggctgatgatcactgcttcacctacgagtatgagcatgtggatgagtccatggaagcaatagcagccctgttcttgcctacccttgacaccgactccgccaacttctcctctagctgtttcaactatgctgtccctccacagtgctggcctcagccagaccatagctctagcgttaccagtttgcttgatccagccgagaactttgagtttccagtcagggacccgctccccccaagcggcttcgatccacattgcgctgtcgcctacctcactgaggattcgagccctctgcatggcaaacgttcatcagtcattgaggaagaagcagccaacgccgcacctgctgctaagaagaggaaggctggtgctgcaatgcagggatcaaagaaatccaggaaggcgagcaaaaaggataacatcggcgacgccgacgatgatggcggctatgcctgtgttgacacgcaaagctccagtagctgcacctccgaggacgggaacttcgaaggaaatacgaattcaagctccaagaagacctgcgccagggccagccgcggagcagcaactgaacctcagagtctctatgcaaggaagaggagagagaggatcaacgaaaggttgagaatcttgcagaacttggttccaaatggaacaaaagtagacattagcacgatgctcgaggaagcggcgcagtatgtcaagtttttacagctccagattaagctgttgagctgtgacgacacatggatgtatgcgccaatcgcgtacaatggaattaacatcggcaatgttgatctgaacatctactctctgcaaaagtaa

SEQ ID NO: 114 - protein, 307 aa, Zea mays
  MOD_RES (7)..(7): Ile or Val
MEDGGLXSEAGAWAELGTGGDESEELVAQLLGAFFRSHGEEGRHQLLWSDDQASSDDVHGDGSLAVPLAYDGCCGYLSYSGSNSDELPLGSSSRAAPAGGPPEELLGAAETEYLNNVAAADHPFFKWCGNGEGLDGPTSVVGTLGLGSGRKRARKKSGDEDEDPSTAIASGSGPTSCCTTSDSDSNASPLESADAGARRPKGNENARAAGRGAAAATTTTAEPQSIYARVRRERINERLKVLQSLVPNGTKVDMSTMLEEAVHYVKFLQLQIRVLQLLSSDDTWMYAPIAYNGMGIGIDLRMHGQDR

SEQ ID NO: 115 - DNA, 924 bp, Zea mays
atggaggacggagggttgrtcagcgaggccggcgcctgggccgagctcggcaccggcggcgacgagtcggaggagctggtggcgcagctgctgggcgccttcttccggtcccacggcgaggaaggccggcaccagctgctttggtctgacgaccaagcttcttccgacgacgtgcacggcgacggcagccttgccgtgccgctcgcatacgacggctgctgcggctatctgagctactcaggtagcaactcggacgagctccccctcgggagcagctcccgcgctgcgccagcaggtggcccaccggaggagctgctcggtgcagctgagactgagtacctgaataatgtggccgccgcagaccatcccttcttcaaatggtgtgggaatggtgagggtctggatggtccgacgagcgtcgtgggcacgcttgggcttggctcgggccggaaacgcgcgcgcaagaagagcggggacgaagacgaagacccgagcacggccatcgccagcggaagcggccccacgagctgctgcactacctccgacagcgactcaaacgcgtctcctctggagtccgcggacgccggcgctcgtcgccccaagggcaacgagaatgcccgggcagctggccgcggcgcggcggcggcgacgacgacgacagcggagccccagagcatctacgcaagggtacggagggagcggatcaacgagaggctcaaggtgctgcagagcctggtgcccaacggcaccaaggtggacatgagcaccatgctcgaggaggccgtccactacgtcaagttcctgcagcttcagatcagggtgctgcagctcctgagctccgacgacacgtggatgtacgcgcccatcgcgtacaacgggatgggcatcgggatcgacctccgcatgcatggacaggacagatga

SEQ ID NO: 116 - protein, 141 aa, Zea mays
SKKSRKASKKDCIVDDDDVYVDPQSSGSCTSEEGNFEGNTYSSAKKTCTRASRGGATDPQSLYARKRRERINERLRILQNLVPNGTKVDISTMLEEAAQYVKFLQLQIKLLSSDDMWMYAPIAYNGINISNVDLNIPALQK

SEQ ID NO: 117 - DNA, 414 bp, Zea mays
tcaaagaaatccaggaaggcgagcaaaaaagattgtattgtcgatgacgacgatgtctatgttgacccgcaaagctccggtagctgcacctccgaggaggggaattttgaagggaatacgtattcaagcgcgaaaaagacctgcaccagggccagccgcggaggagcaactgatcctcagagtctctatgcaaggaagaggagagagaggatcaatgaaaggttgagaatcttgcagaacttggtccccaatggaacaaaggttgacattagtacgatgctcgaggaagcagcacagtatgtcaaatttttacagcttcagattaagctgttgagctctgacgacatgtggatgtatgcgccaatcgcgtacaatgggatcaacatcagcaatgttgatctgaacatccctgca

(19) United States
(12) Patent Application Publication
(10) Pub. No.: US 2013/0081153 A1
(43) Pub. Date: Mar. 28, 2013

(54) Expression Of Transcription Factor Encoding Genes

(71) Applicant: Liam Dolan, Oxford (GB)

(72) Inventor: Liam Dolan, Oxford (GB)

(21) Appl. No.: 13/562,850

(22) Filed: Jul. 31, 2012

(30) Foreign Application Priority Data
Aug. 5, 2011 (GB) 1113499.6

(51) Int. Cl. C12N 15/82

(52) U.S. Cl. CPC C12N 15/8261
USPC 800/278; 435/320.1; 435/419; 800/298; 435/468

(57) ABSTRACT

Constructs, vectors and methods that facilitate the constitutive expression of transcription factor encoding genes in specific cell types are described.

### RELATED APPLICATIONS AND INCORPORATION BY REFERENCE

This application claims benefit of UK patent application Serial No. 1113499.6 filed 5 Aug. 2011.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to the field of molecular engineering and providing systems and compositions for gene expression in an organism.

### INTRODUCTION

Transcription factors control gene expression by interacting with a gene sequence, such as a promoter regulatory sequence. The interaction may be direct sequence-specific binding and the transcription factor directly contacts the gene or gene regulatory sequence. Alternatively, the transcription factor may interact with other proteins to control gene expression. In some cases, the binding and/or effect of one transcription factor is influenced (in an additive, synergistic or inhibitory manner) by another transcription factor.

Manipulation of the expression of transcription factors allows for manipulation of downstream gene expression of target genes of interest as expression of the transcription factor will affect downstream gene expression. Thus, through constitutive gene expression of a transcription factor, downstream gene expression of a gene of interest can also be enhanced.

Promoters that confer constitutive expression in various organisms are known. In plants, the 35S promoter from cauliflower mosaic virus has been widely used. Promoters from other viruses have also been shown to confer similar activity. Whilst constitutive expression of a transgene driven by the 35S promoter is not limited to a specific tissue, it is often desirable to target gene expression to certain sites within an organism and this can be achieved through the use of tissue specific promoters.

The present invention provides alternative means for constitutive expression of a transcription factor in a cell, tissue or organ where it is normally expressed as well as in a cell, tissue or organ where it is not normally expressed.

Citation or identification of any document in this application is not an admission that such document is available as prior art to the present invention.

### SUMMARY

The invention relates to constructs, vectors, systems and methods for constitutive expression of a transcription factor gene by creating a positive feedback loop of expression. Thus, it relates to constitutive expression of transcription factor (TF) encoding genes in a cell, tissue or organism using target gene promoter-transcription factor (TART) fusions. In this way, the expression of the downstream target gene may be increased. In one aspect, the invention relates to an expression construct for constitutive expression of a transcription factor gene which may comprise an isolated nucleic acid sequence encoding a transcription factor operably linked to an isolated promoter nucleic acid sequence wherein said promoter sequence is derived from the promoter sequence of a target gene of said transcription factor and wherein said transcription factor regulates expression of said target gene.

In another aspect, the invention relates to a vector which may comprise an expression construct as described above. Also within the scope of the invention is a host cell expressing such a vector or construct and the use of an expression construct described above for constitutive expression of a transcription factor gene.

In another aspect, the invention relates to a method for constitutive expression of a transcription factor gene which may comprise introducing the expression construct which may comprise an isolated nucleic acid sequence encoding a transcription factor operably linked to an isolated promoter nucleic acid sequence into a host cell or organism wherein said promoter sequence is derived from the promoter sequence of a target gene of said transcription factor and wherein said transcription factor regulates expression of said target gene.

In a further aspect, the invention relates to a method for constitutive expression of a transcription factor gene which may comprise introducing into a host cell or organism a first expression construct which may comprise an isolated nucleic acid sequence encoding a transcription factor gene operably linked to an isolated promoter nucleic acid sequence wherein said promoter sequence is derived from the promoter sequence of a target gene of said transcription factor and wherein said transcription factor regulates expression of said target gene and introducing a second expression construct into said host cell or organism wherein said second expression construct may comprise an isolated nucleic acid sequence encoding said transcription factor operably linked to a second isolated promoter nucleic acid sequence specific to a cell, tissue or organ in which said transcription factor is not normally expressed.

Thus, in one aspect, the invention relates to methods for differential gene expression. These methods comprise constitutive expression of a gene in a tissue or organ where it is not normally expressed.

The organism according to all of the aspects of the invention is prokaryotic or eukaryotic. In a preferred embodiment, the organism is a plant and the nucleic acid sequences described herein are derived from plants.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Schematic representation of transcription factor (T) genes, their target (TAR) genes and a TAR-T gene fusion.

FIG. 2. Schematic representation of RSL4 transcription factor gene, its target (EXP7) genes and a EXP7-RSL4 gene fusion.

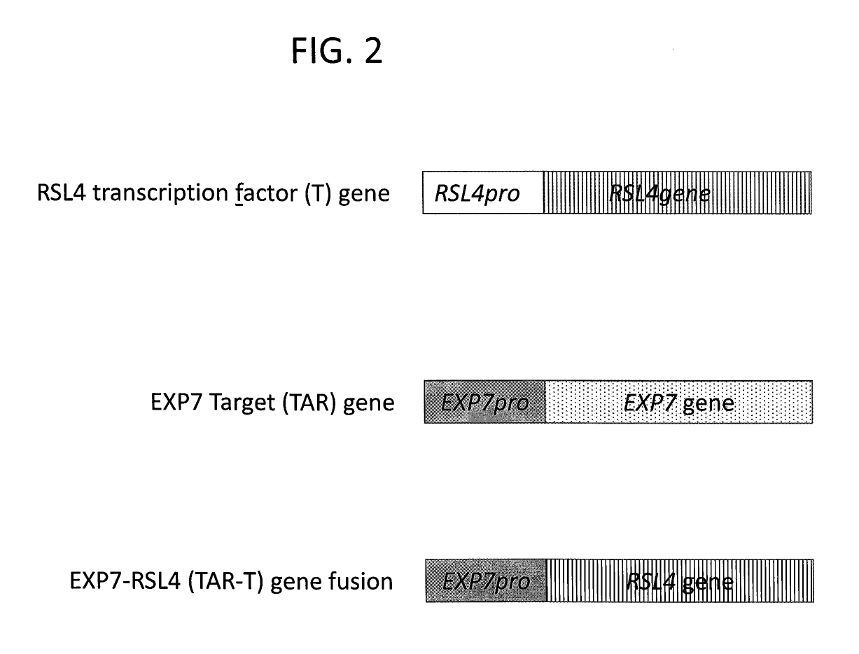

FIG. 3. A: Schematic gene expression in a non-transformed organism; B: positive transcriptional feed back resulting from fusing a target promoter (TAR) to the transcription factor (T) that regulates its transcriptional activity.

FIG. 4. A: Gene expression in a non-transformed Arabidopsis root hair cell; B: positive transcriptional feed back resulting from fusing a target promoter (EXP7) to the RSL4 gene, which controls transcription from the EXP7 promoter.

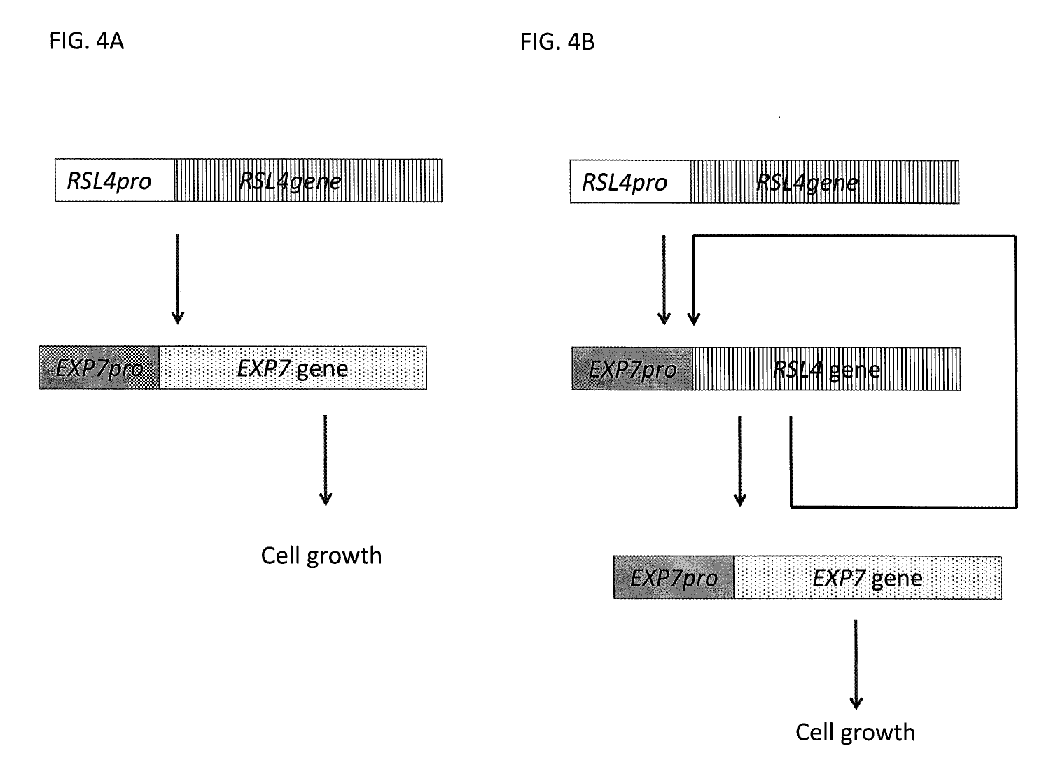

FIG. 5. A (left hand side): wild type plants; B (right hand side): Plants transformed with EXP7:RSL4 transgene C: Plants transformed with 35S:RSL4 transgene.

FIG. 6. A (left hand side): wild type plants; B (right hand side): Plants transformed with 35S:RSL4 transgene.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of one in the art. Such techniques are explained fully in the literature.

The present invention relates to a chimeric/heterologous gene or expression construct which may comprise an isolated polynucleotide sequence operably linked to an isolated promoter nucleic acid sequence. The nucleic acid sequence is “heterologous” or “chimeric” with respect to the promoter sequence as this promoter sequence does not function in nature, i.e. in a wild type organism, to regulate the expression of the transcription factor gene.

Transcriptional activation of genes, including transgenes, is in general controlled by a promoter sequence through a complex set of protein/DNA and protein/protein interactions. Promoters are regulatory sequences that may impart patterns of expression that are either constitutive or limited to specific tissues or times during development. As used herein, the term “promoter” refers to a nucleic acid sequence that functions to direct transcription of a gene. A promoter sequence may comprise binding sites for a protein which regulates transcription of the downstream gene.

Thus in a first aspect, the invention relates to an expression construct for constitutive expression of a transcription factor gene which may comprise an isolated nucleic acid sequence encoding a transcription factor operably linked to an isolated promoter nucleic acid sequence wherein said promoter sequence is derived from the promoter sequence of a target gene of said transcription factor and wherein said transcription factor regulates expression of said target gene. The transcription factor gene thus encodes a protein that interacts with said promoter sequence or interacts with another protein which in turn interacts with the promoter sequence to direct the expression of a downstream target gene. Thus, the transcription factor upregulates its own expression in a positive feedback loop. The promoter and transcription factor nucleic acid sequences are preferably, as described herein, both endogenous to the organism in which the expression construct of the invention is expressed, but in a wild type organism, they are not operably linked.

The transcription factor regulates expression of said target gene from which the promoter is derived. This may be directly or indirectly, for example the transcription factor may bind directly to the promoter or indirectly. In one embodiment, the transcription factor positively regulates expression of said target gene indirectly. For example, the transcription factor binds to the promoter of another gene that encodes a proteins that in turn binds to the promoter.

The downstream target gene is a gene endogenous to the organism and not a further transgene.

As used herein, the term “gene” means the segment of DNA involved in producing a polypeptide chain, which may or may not include regions preceding and/or following the coding region, e.g. 5′ untranslated (5′UTR) or “leader” sequences and 3′UTR or “trailer” sequences, as well as intervening sequences (introns) between individual coding segments (exons). The term “gene” may be used interchangeably herein with the terms “isolated nucleic acid sequence” and “isolated polynucleotide”. The gene has a sequence which encodes a transcription factor and is thus a polynucleotide which may comprise the coding sequence of the transcription factor (i) in isolation, (ii) in combination with additional coding sequences, such as fusion protein or signal peptide, in which the transcription factor coding sequence is the dominant coding sequence, (iii) in combination with non-coding sequences, such as control elements and terminator elements, effective for expression of the coding sequence in a cell.

An increase in gene expression as used herein may be at least 10%, at least 20%, at least 30%, at least 40%, at least 50% or more.

As used herein, the term “operably linked” means that the promoter nucleic acid sequence and transcription factor nucleic acid sequence of the expression construct are in a functional relationship with each other. Thus, the promoter is operably linked to the transcription factor nucleic acid sequence if it affects the transcription of said transcription factor nucleic acid sequence.

As explained in more detail below, the expression construct described herein may, when introduced into a host cell or organism, be used to achieve constitutive expression of a transcription factor gene through a positive feedback loop in a host cell, tissue or organ in which the transcription factor gene is normally expressed. Thus, the nucleic acid encoding a transcription factor gene is preferably a nucleic acid which encodes a transcription factor that is expressed in a specific cell, tissue or organ and/or under specific conditions in a wild type organism.

Furthermore, it is also preferred that the isolated promoter nucleic acid sequence used in the expression construct is a cell, tissue or organ specific promoter and/or regulates gene expression under specific conditions, for example environmental conditions. Thus, in one embodiment, the promoter directs the expression of a downstream target gene of the transcription factor in the same cell, tissue or organ in which the transcription factor gene is normally expressed. Therefore, in a preferred embodiment, the expression construct described herein may be used according to the methods of the invention to drive the expression of the transcription factor gene in those cells, tissues or organs where the transgene product is desired and normally expressed, leaving other cells, tissues or organs unmodified by transgene expression. This is advantageous over the use of expression constructs that use constitutive promoters such as CaMV35S to achieve constitutive expression because using the constructs of the invention, expression may be spatially regulated. Moreover, using developmentally regulated promoters, the timing of gene expression may also be regulated.

As used herein, the term “expression” refers to the process by which a polypeptide is produced based on the nucleic acid sequence of a gene. The process generally includes both transcription and translation.

In one embodiment, the expression construct(s) described herein includes other transcriptional and translational regulatory sequences such as, but not limited to, ribosomal binding sites, transcriptional start and stop sequences, translational start and stop sequences, elements that are responsive to certain environmental conditions, such as heat shock elements, and enhancer, control, terminator or activator sequences. In one embodiment, the vectors and constructs of the invention do not comprise any additional regulatory sequence.

According to the invention, the promoter and transcription factor nucleic acid sequences are both derived from the same type of organism, preferably from the same species. For example, in one embodiment, the promoter and transcription factor nucleic acid sequences are both derived from a prokaryotic organism. In another embodiment, the promoter and transcription factor nucleic acid sequences are both derived from a eukaryotic organism. Examples of a prokaryotic organism are gram-negative bacteria, including E. coli, and gram-positive bacteria. The eukaryotic organism may be yeast, an animal, or a plant. In one embodiment, the eukaryotic organism is an animal, for example a mammal, such as a rodent. In one embodiment, the animal may be a mouse. In a preferred embodiment, the eukaryotic organism is a plant.

As will be immediately apparent to the skilled person, the methods described herein may be used in any type of organism and expression construct for use in an organism of interest and may be designed accordingly. Many transcription factors and their target genes are known in a wide range of organisms and a skilled person would be able to select a transcription factor that targets a gene of interest to manipulate the expression of the target gene and use said sequence to obtain an expression construct according to the invention.

Also within the scope of the invention are artificial promoters that have been specifically designed to not only include sequences to which the specific transcription factor or another protein whose expression is regulated by the transcription factor binds, but also include other sequence features, such as binding sites for inducers etc.

In a preferred embodiment of the different aspects of the invention, the eukaryotic organism is a plant. Thus, in one embodiment, the plant promoter is operably linked to a plant transcription factor gene. A typical plant transcription factor gene may comprise a DNA-binding region, an oligomerization site, a transcription-regulation domain and a nuclear localization signal. Most plant transcription factors exhibit only one type of DNA-binding and oligomerization domain, occasionally in multiple copies, but some contain two distinct types. DNA-binding regions are normally adjacent to or overlap with oligomerization sites, and their combined tertiary structure determines critical aspects of transcription factor activity.

Thus, in this embodiment of the invention, the plant promoter operably linked to a plant transcription factor gene is derived from a downstream target gene of the transcription factor and therefore also a plant sequence, preferably from the same plant species. The promoter used in the constructs of the invention is preferably cell, tissue or organ specific and/or regulates expression during certain developmental stages or under specific conditions, such as biotic or abiotic stress. The transcription factor may direct the expression of the transcription factor in any specific plant tissue or organ, including reproductive and non-reproductive organs. For example, expression may be targeted to in a tissue selected from the following non-limiting list: root, meristem, flower, seed, pollen, embryo, leaf, stem or fruit.

Plant transcription factor classes are known to the person skilled in the field. For example, a non-limiting list of transcription factor families in the model plant Arabidopsis thaliana is shown below (from Riechmann and Ratcliff, 2000). A skilled person would know that TFs in Arabidopsis thaliana have orthologues in other plant species, including monocot crop plants. This is described in the art.

### Table 1. Non-Limiting List of Transcription Factor Families in Arabidopsis

MYB (involved in secondary metabolism, cellular morphogenesis, signal transduction in plant growth, abiotic and biotic stress responses, circadian rhythm and dorsoventrality). This family includes genes such as AtMYB2, ATR1, CCA1, CPC, GL1, LHY, WER. 198 genes in the MYB superfamily from Arabidopsis have been identified in an analysis of the complete Arabidopsis genome sequence, among them, 126 are R2R3-MYB, 5 are R1R2R3-MYB, 64 are MYB-related, and 3 atypical MYB genes (Yanhui et al, Dubos et al).
AP2/EREBP (involved in development, cell proliferation, secondary metabolism, abiotic and biotic stress responses, hormone signalling). AP2 (APETALA2) and EREBPs (ethylene-responsive element binding proteins) are the prototypic members of a family of transcription factors unique to plants, whose distinguishing characteristic is that they contain the so-called AP2 DNA-binding domain. AP2/REBP genes form a large multigene family, and they play a variety of roles throughout the plant life cycle: from being key regulators of several developmental processes, like floral organ identity determination or control of leaf epidermal cell identity, to forming part of the mechanisms used by plants to respond to various types of biotic and environmental stress. AP2/EREBP genes are divided into two subfamilies: AP2 genes with two AP2 domains and EREBP genes with a single AP2/ERF (Ethylene Responsive Element Binding Factor) domain. Expressions of AP2-like genes, including AP2, in Arabidopsis thaliana are regulated by the microRNA miR172. The target site of miR172 is significantly conserved in gymnosperm AP2 homologs, suggesting that regulatory mechanisms of gene expression using microRNA have been conserved over the three hundred million years since the divergence of gymnosperm and flowering plant lineages. Members of this family possess an AP2 domain. In the A. thaliana transcription factor RAV1 the N-terminal AP2 domain binds a 5′-CAACA-3′ sequence, while the C-terminal highly conserved B3 domain binds a 5′-CACCTG-3′ sequence. There are orthologues in, for example, Oryza sativa subsp. Indica, Oryza sativa subsp. Japonica, Sorghum bicolor, Zea mays and Populus trichocarpa.

This family includes genes such as ABI4, ANT, AP2, CBF1-3/DREB1A-C, DREB2A, ERF transcription factors, such as ERF1 (Riechmann et al, 1998).
NAC (involved in development, pattern formation and organ separation, stress response). This family includes genes such as CUC2, NAP, NAC SECONDARY WALL THICKENING PROMOTING FACTOR1 (NST1) and NST3 and, in rice, OsNAC6 (Olsen et al).
bHLH/MYC (involved in anthocyanin biosynthesis, light response, flower development, formation of secondary cell walls and abiotic stress). There are 133 bHLH genes in Arabidopsis thaliana and at least 113 of them are expressed. The AtbHLH genes constitute one of the largest families of transcription factors in A. thaliana with significantly more members than are found in most animal species and about an equivalent number to those invertebrates. Comparisons with animal sequences suggest that the majority of plant bHLH genes have evolved from the ancestral group B class of bHLH genes. By studying the AtbHLH genes collectively, twelve subfamilies have been identified. Within each of these main groups, there are conserved amino acid sequence motifs outside the DNA binding domain. Typically, a bHLH domain may comprise a stretch of about 18 hydrophilic and basic amino acids at the N-terminal end of the domain, followed by two regions of hydrophobic residues predicted to form amphipathic α helices. separated by an intervening loop. This family includes genes such as PIFs, e.g. PIF3 (Heim et al).
bZIP (involved in seed-storage gene expression, photomorphogenesis, leaf development, flower development defense response, ABA response, and gibberellin biosynthesis). The Arabidopsis genome sequence contains 75 distinct members of the bZIP family, This family includes genes such as ABI5, HY5, PAN. Members are also known for example in rice (Nijhawan et al) and soybean. These include root and vascular specific TFs.
HB or HD-Zip proteins (involved in leaf, root, internode development, stem cell identity, cell anthocyanin accumulation, and cell death differentiation, growth responses). This family includes genes such as ANL2, ATHB-2, BEL1, GL2, KNAT1, REV, STM, WUS.

HD-Zip proteins characterized by the presence of a homeodomain associated with a leucine zipper constitute one family of plant transcription factors. The association of the DNA binding domain (HD) with an adjacent dimerization motif (leucine zipper abbreviated ZipLZ or LZ) is a combination found only in the plant kingdom, although the domains are found independently of each other in a large number of eukaryotic transcription factors. This large family of plant TFs has been divided into four subfamilies (I to IV) according to sequence similarity in and outside the conserved domains and by the intron/exon patterns of the corresponding genes. Members of subfamily I interact with the pseudopalindromic sequence CAAT(A/T)ATTG; subfamily II proteins recognize a motif CAAT(C/G)ATTG. In all cases, the formation of protein homo- or hetero-dimers is a prerequisite for DNA binding. Members of the HD-Zip family exhibit a LZ motif just downstream from the HD motif. The two motifs are present in transcription factors belonging to other eukaryotic kingdoms, but their association with each other in a single protein is unique to plants. The HD is responsible for the specific binding to DNA while the LZ acts as a dimerization motif. HD-Zip proteins bind to DNA as dimers, and the absence of the LZ absolutely abolishes their binding ability, indicating that the relative orientation of the monomers, driven by this motif, is crucial for an efficient recognition of DNA.

In Arabidopsis, subfamily I is composed of seventeen members (ATHB1/HAT5, 3/HAT7, 5, 6, 7, 12, 13, 16, 20, 21, 22, 23, 40, 51, 52, 53, 54). HD-Zip I subsets of genes (in Arabidopsis) share their intron/exon distribution in accordance with their phylogenetic relationships. The molecular weight of the encoded proteins is about 35 kDa and exhibit a highly conserved HD and a less conserved LZ. There are numerous homologs and orthologs in other plants.
Z-C₂H₂ (involved in flower development, flowering time, seed development, and root

nodule development). This family includes genes such as FIS2, SUP 352 (Englebrecht et al).
MADS (involved in flower development, fruit development, flowering time and root development). MADS-box transcription factors are key regulators of several plant development processes. Analysis of the complete Arabidopsis genome sequence revealed 107 genes encoding MADS-box proteins, of which 84% are of unknown function. These are divided into five groups (named MIKC, Mα, Mβ, Mγ, Mδ) based on the phylogenetic relationships of the conserved MADS-box domain.

The MIKC type has a characteristic modular structure. From the N- to the C-terminus of the protein, four characteristic domains may be identified: the MADS-box (M), intervening (I), keratin-like (K), and C-terminal (C) domains. The MADS-box is a DNA binding domain of about 58 amino acids that binds DNA at consensus recognition sequences known as CArG boxes [CC(A/T)₆GG]. The interaction with DNA has been studied in detail for the human and yeast MADS-box proteins thanks to the resolved crystal structures. The I domain is less conserved and contributes to the specification of dimerization. The K domain is characterized by a coiled-coil structure, which facilitates the dimerization of MADS-box proteins. The C domain is the least conserved domain; in some cases, it has been shown to contain a transactivation domain or to contribute to the formation of multimeric MADS-box protein complexes.

This family includes genes such as AG, AGL15, ANR1, AP1, AP3, CAL, FLC, FUL, PI, SEP1, SEP2, SEP3, SHP1, SHP2, SOC1, SVP (Parenicová et al).
WRKY (involved in defence response and immunity). The WRKY family proteins contain one or two highly conserved WRKY domains characterized by the hallmark heptapeptide WRKYGQK and a zinc-finger structure distinct from other known zinc-finger motifs. To regulate gene expression, the WRKY domain binds to the W box in the promoter of the target gene to modulate transcription. In addition to the W box, a recent study indicates that the WRKY domain may also bind to SURE, a sugar responsive cis element, as a transcription activator. Members of the WRKY superfamily from the Arabidopsis genome are classified into three groups. Members of Group 1 typically contain two WRKY domains, while most proteins with one WRKY domain belong to Group 2. Group 3 proteins also have a single WRKY domain, but the pattern of the zinc-finger motif is unique (Zhang et al).
ARF-Aux/IAA (involved in auxin responses, development and floral meristem patterning). Aux/IAA proteins are short-lived nuclear proteins that repress expression of primary/early auxin response genes in protoplast transfection assays. Repression is thought to result from Aux/IAA proteins dimerizing with auxin response factor (ARF) transcriptional activators that reside on auxin-responsive promoter elements, referred to as AuxREs. Most Aux/IAA proteins contain four conserved domains, designated domains I, II, III, and IV. Domain II and domains III and IV play roles in protein stability and dimerization, respectively domain I in Aux/IAA proteins may be an active repression domain that is transferable and dominant over activation domains. An LxLxL motif within domain I is important for conferring repression. The dominance of Aux/IAA repression domains over activation domains in ARF transcriptional activators provides a plausible explanation for the repression of auxin response genes via ARF-Aux/IAA dimerization on auxin-responsive promoters.

This family includes genes such as AXR2, AXR3, ETT, MP, NPH4, SHY2. (Tiwari et al)
Dof (involved in seed germination, endosperm-specific expression, and carbon metabolism. This family includes genes such as DAG1 (Yanagisawa et al).
Heat shock transcription factors (Hsfs) that act by binding to a highly conserved palindromic heat shock response sequence in the promoters of the target genes. In addition to mediating the response to heat stress, Hsfs are thought to be involved in cellular responses to oxidative stress, heavy metals and other stress responses. It is known that the basic structure of Hsfs and of their promoter recognition site is conserved throughout the eukaryotic kingdom. Hsfs have a modular structure with a highly conserved N-terminal DNA binding and a C-terminal activation domain. Other conserved domains include an oligomerisation domain, a nuclear localisation sequence and a nuclear export sequence. Thus, Hsfs are easily recognised by their conserved motifs essential for their function as transcription factors. Plant Hsfs are divided into three groups A, B and C (see WO2008/110848).

A skilled person would know that the application is applicable to any transcription factor, specifically any plant transcription factor. A skilled person would also know that many of the families as listed above have homologues and orthologues in other plant species. Any transcription factor within those families above or a homologue and orthologue thereof may be used according to the various aspects of the invention.

Plant transcription factors regulate many developmental and physiological processes and by using the constructs and methods of the invention, these may be altered through constitutive expression of the selected transcription factors involved in said process. Preferably, the transcription factor is involved in the regulation of pathways of agronomic interest. These pathways may concern plant morphology, physiology, growth, development, yield, control of metabolism, nutritional profile, stress resistance, such as disease or pest resistance, and/or environmental or chemical tolerance. Expression of the constructs described herein and the methods of the invention may therefore be used to enhance or confer a beneficial trait compared to a control plant, for example a wild type plant, which does not express the expression construct or vector according to the invention which has been introduced as a transgene into said organism.

A beneficial trait may be, but is not limited to: increased growth/yield, herbicide tolerance, insect control, fungal disease resistance, virus resistance, nematode resistance, bacterial disease resistance, modified plant development, starch production, modified oil production, modified fatty acid content, modified fruit ripening, enhanced value for animal and human nutrition, environmental stress resistance, improved flavour, increased seed storage protein content, modified plant architecture, increased root formation, modified metabolite content or improved nitrogen fixation. Developmental and physiological processes that may be targeted to achieve a benefit include: root formation, flowering time, seed development, senescence, metabolite production, hormone production/signalling or stress tolerance. Stress tolerance may be tolerance again biotic or abiotic stress, for example draught, pathogen invasion, cold, freezing, deficit of nutrients in the soil, heat or other types of stress.

In one embodiment, the beneficial trait relates to an improvement of root architecture. Improved root architecture may be selected from a non exclusive list of altered diameter, length, weight, number, angle or surface of one or more of the root system parts, including but not limited to, the primary root, lateral or branch root, adventitious root, and root hairs, all of which fall within the scope of this invention. These changes may lead to an overall alteration in the area or volume occupied by the root. In one embodiment, growth of root hairs is altered. This is achieved by constitutive expression of an expansin gene, for example EXP7. Expansin refers to a family of closely related nonenzymatic proteins found in the plant cell wall, with important roles in plant cell growth, fruit softening, abscission, emergence of root hairs, pollen tube invasion of the stigma and style, meristem function, and other developmental processes where cell wall loosening occurs. Where a feature is of the root is increased, the increase may be at least 10%, at least 20%, at least 30%, at least 40%, at least 50% or more. In one embodiment, the altered root phenotype is increased or length. The increase may be at least 10%, at least 20%, at least 30%, at least 40%, at least 50% or more. In one embodiment, then total mass/weight of the root is increased. The increase may be at least 10%, at least 20%, at least 30%, at least 40%, at least 50% or more.

The root phenotype is altered compared to a control plant. A control plant as used according to the different aspects of the invention is a plant, which has not been modified according to the methods of the invention. Accordingly, the control plant has not been genetically modified to express a nucleic acid as described herein to alter the root phenotype. In one embodiment, the control plant is a wild type plant. In another embodiment, the control plant is a plant that does not carry a transgenic according to the methods described herein, but expresses a different transgene. The control plant is typically of the same plant species, preferably the same ecotype as the plant to be assessed.

The term “yield” as described herein relates to yield-related traits. Specifically, these include an increase in biomass and/or seed yield. This may be achieved by increased growth. An increase in yield may be, for example, assessed by the harvest index, i.e. the ratio of seed yield to aboveground dry weight. Thus, according to the invention, yield may comprise one or more of: increased seed yield per plant, increased seed filling rate, increased number of filled seeds, increased harvest index, increased number of seed capsules/pods, increased seed size, increased growth or increased branching, for example inflorescences with more branches. Preferably, yield may comprise an increased number of seed capsules/pods and/or increased branching. Yield is increased relative to control plants. An increase in yield may be about 5, 10, 20, 30, 40, 50% or more compared to a control plant. A control plant is a plant that does not express a construct or vector as described herein. The plant may be a wild type plant or a plant which has been genetically modified in another way.

The plant transcription factor gene may be selected from any of the examples in table. 1. In one embodiment, the plant transcription factor gene may for example be selected from RSL4, SND, GL1, MP, ARF7, AGL28, Cr1, WRI1, Opaque2, KN, OCL1, DREB1 or a homologue or orthologue thereof. In one embodiment, the plant transcription factor gene is RSL4 (SEQ ID NO. 2) or a homologue or orthologue thereof. Thus, any ROOT HAIR DEFECTIVE 6 (RHD6)-related gene or RHD6 may be used. RHD6-related genes include genes capable of complementing the rhd6 mutation in plants. Thus, the RSL4 homologue or orthologue thereof may be selected from any of the nucleic acid/amino acid sequences SEQ ID No. 5 to 117. RSL4 or a homologue or orthologue thereof are disclosed in WO 2008/142364. RSL4 or any homologue or orthologue may be expressed using EXP7.

The plant promoter may be selected from any promoter which is a promoter of a downstream target gene of the transcription factor selected. In a preferred embodiment, the promoter is a tissue or organ specific promoter. In another preferred embodiment, the promoter is developmentally regulated.

A preferred tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the aspects of the present invention which causes the desired temporal and spatial expression.

The promoter may be specific to any organ of the plant, including reproductive organs and a non-limiting list includes roots, including parts thereof such as root trichomes, seeds, stems, leaves, fruits, flowers or parts thereof, stems, rhizomes, tubers, embryos and bulbs. The promoter may direct tissue specific expression, for example expression in meristems, parenchyma, collenchyma or sclerenchyma.

Promoters which are seed or embryo specific and may be useful in the invention include soybean Kunitz trysin inhibitor, patatin (potato tubers), convicilin, vicilin, and legumin (pea cotyledons), zein (maize endosperm), phaseolin (bean cotyledon), phytohemagglutinin (bean cotyledon), B-conglycinin and glycinin (soybean cotyledon), glutelin (rice endosperm), hordein (barley endosperm), glutenin and gliadin (wheat endosperm) and sporamin (sweet potato tuberous root).

Plant root systems are essential for crops to capture water and nutrients for growth and yield. There is a positive correlation between the size of the plant root system and greater capture of water and nitrogen and grain-fill. In many environments, water uptake may be a limiting factor for crop yield. Thus, in another embodiment, a root-specific promoter may be used. This is a promoter that is transcriptionally active predominantly in plant roots, substantially to the exclusion of any other parts of a plant, whilst still allowing for any leaky expression in these other plant parts. Examples of promoters specific to roots or part thereof according to the various aspects of the invention include promoters of root expressible genes, for example the promoters of the following genes: RCc3, Arabidopsis PHT1, Medicago phosphate transporter, Arabidopsis Pyk10, tobacco auxin-inducible gene, beta-tubulin, LRX1, ALF5, EXP7, LBD16, ARF1, tobacco RD2, S1REO, Pyk10, PsPR10.

Root hairs play important roles in plant nutrition and water uptake. In most soils they are important for phosphate and iron uptake. In drought conditions they are important in the uptake of other nutrients such as nitrate. Therefore the manipulation of root hair traits will be important in developing crops that may effectively extract nutrients from the soil. In one embodiment, the promoter is specific to root hairs. In a preferred embodiment, the promoter is EXP7 (SEQ ID NO. 1).

One non-limiting embodiment of the first expression according to the various aspects of the invention is shown in example 1. This shows an expression construct (EXP7pro-RSL4) which enables constitutive expression of the plant transcription factor RSL4 in root hairs cells through a positive feedback loop. This in turn activates expression of the RSL4 downstream target EXP7. The construct is expressed in root hair cells where RSL4 is naturally expressed. The introduction and expression of the expression construct results in constitutive expression of RSL4. This in turn increases expression of the downstream target gene EXP7. Transgenic plants expressing said construct develop longer root hairs compared to wild type plants.

Other non-limiting examples of genes and constructs that are within the scope of the various aspects of the invention are set out in table 2. A skilled person would appreciate that homologues and orthologues in other plants may be used.

### Table 2. Non-Limiting Examples of Genes and Constructs

1. Expression of SND in Fibre Cells (Arabidopsis thaliana)
SND is a transcription factor that positively regulates the expression of MYB64 in fibre cells (Zhong et al 2007).
TART construct for constitutive expression in fibre cells: MYB46promoter:SND1
Expected phenotypic consequences: Thinning of cell walls (Zhong et al 2007b).
   2. Expression of GL1 in Trichome Cells (Arabidopsis thaliana)
GL1 is a transcription factor that positively regulates the expression of MYC1, SCL8, SIM and RBR1 genes in trichomes (Morohashi and Grotewold 2010).
TART constructs for constitutive expression in trichomes:
   MYC1promoter:GL1
   SCL8promoter:GL1
   SIMpromoter:GL1
   RBR1promoter:GL1
Expected phenotypic consequences: Reduction in trichomes number.
   3. Constitutive Expression of MP in Embryos (Arabidopsis thaliana)
MP is a transcription factor that positively regulated the expression of TMO5 and TMO7 in embryos (Schlereth et al. 2010).
TART constructs for constitutive expression in embryos:
   TMO5promoter:MP
   TMO7promoter:MP
Expected phenotype: Architectural variation
   4. Constitutive Expression of ARF7 in Lateral Roots (Arabidopsis thaliana)
ARF7 is a transcription factor that positively regulates the expression of LBD16 and LBD18 in lateral roots (Okushima et al 2007).
TART constructs for constitutive expression in lateral roots:
   LBD16promoter:ARF7
   LBD18promoter:ARF7
Expected phenotypic consequences: Increase in the number of lateral roots (Okushima et al 2007).
   5. Constitutive Expression of AGL28 Promotes Flowering (Arabidopsis thaliana)
Constitutive expression of AGL28 promotes flowering by positively regulating expression of FCA and LD.
TART constructs for constitutive expression of AGL28:
   FCApromoter:AGL18
   LDpromoter:AGL18
Expected phenotype: modified flowering time.

### 6. Constitutive Expression of Cr1 During Crown Root Formation in Rice

OsARF1 positively regulates Cr1 during crown root formation in rice (Inukaki et al 2005).
TART construct for constitutive expression of OsARF1:
   Cr1promoter:OsARF1
Phenotypic consequences: increase in crown root number.

### 7. Constitutive Expression of WRI1a in Maize Kernels

WRI1a controls the expression of the following maize genes:
MZ00042142, MZ00024552, MZ00043500, MZ00024718, MZ00016632, MZ00014741, MZ00043050, MZ00056535, MZ00017651, MZ00016866, MZ00017355, MZ00040095, MZ00042163, MZ00016943, MZ00044044, MZ00026553, MZ00015977, MZ00031529, MZ00039375 (Pouvreau et al 2011)
TART constructs for constitutive expression of WR1a:
   MZ00042142promoter:WRI1a
   MZ00024552promoter:WRI1a
   MZ00043500promoter:WRI1a
   MZ00024718promoter:WRI1a
   MZ00016632promoter:WRI1a
   MZ00014741promoter:WRI1a
   MZ00043050promoter:WRI1a
   MZ00056535promoter:WRI1a
   MZ00017651promoter:WRI1a
   MZ00016866promoter:WRI1a
   MZ00017355promoter:WRI1a
   MZ00040095promoter:WRI1a
   MZ00042163promoter:WRI1a
   MZ00016943promoter:WRI1a
   MZ00044044promoter:WRI1a
   MZ00026553promoter:WRI1a
   MZ00015977promoter:WRI1a
   MZ00031529promoter:WRI1a
   MZ00039375promoter:WR1a
Expected phenotype: increases in palmitic acid, succinic acid, linolenic acid, lysine, oleic acid, glyceric acid, stearic acid, citric acid, glutamic acid phosphoric acid, phenylalanine, arabinose, linoleic acid, pyroglutamic acid, norleucine, nicotinic acid, alanine, valine, aminoadipic acid, ornithine content.

### 8. Constitutive Expression of Opaque2 in Maize Endosperm

Opaque2 controls CyPPDK1 22 kd zein proteins encoding genes and p32 protein encoding genes in maize endosperm (Gallusci et al 1996; Maddoloni et al 1996).
TART constructs for constitutive expression of Opaque2:
   CyPPDK1promoter:Opaque2
   Zeinpromoter:Opaque2
   Protein32promoter:Opaque2
Expected phenotype: increased seed storage protein content.

### 9. Constitutive Expression of Knotted (KN) Gene in Maize Meristems

KN1 gene positively regulates the expression of GA2OX1 in maize (Bolduc and Hake, 2009).
TART constructs for constitutive expression of KN1 in maize:
   GA2OX1promoter:KN1
Expected phenotype: modified shoot architecture

### 10. Constitutive Expression of OCL1 in Maize

OCL1 positively regulated the expression of ZmWBC11b, ZmWBC11c, ZmLtpII.12, ZmFAR1, MZ00030315, MZ00029474, MZ00022171, and MZ00031955 (Javelle et al 2010).
TART constructs for constitutive expression of OCL1 in maize:
   ZmWBC11b:promoterOCL1
   ZmWBC11c:promoterOCL1
   ZmLtpII.12:promoterOCL1
   ZmFAR1:promoterOCL1
   MZ00030315:promoterOCL1
   MZ00029474:promoterOCL1
   MZ00022171:promoterOCL1
   MZ00031955:promoterOCL1
Expected phenotype: Modified cuticle and kernel.

### 11. Constitutive Expression of DREB1 in Rice

DREB1 positively regulates the expression of J033041J03, J013078A14, 001-120-D04, J013091D15, J023041L05, J023082D02, J013097O21, 001-125-G03, 001-104-B03, 001-023-B08, J023121A17 and J023042N13 genes in rice (Ito, et al., 2006).
TART constructs for constitutive expression of DREB1 in rice
   J033041J03:promoterDREB1
   J013078A14:promoterDREB1
   001-120-D04:promoterDREB1
   J013091D15:promoterDREB1
   J023041L05:promoterDREB1
   J023082D02:promoterDREB1
   J013097021:promoterDREB1
   001-125-G03:promoterDREB1
   001-104-B03:promoterDREB1
   001-023-B08:promoterDREB1
   J023121A17:promoterDREB1
   J023042N13:promoterDREB1
Expected phenotype: enhanced stress resistance.

Thus, any construct disclosed in table 2 may be used according to the different aspects and embodiments of the invention described herein.

In another aspect, the invention relates to a vector which may comprise a first expression construct as described herein. As used herein, the term “vector” refers to a nucleic acid construct designed for transfer between different host cells. It has the ability to incorporate and express heterologous DNA fragments in a foreign cell. Many prokaryotic and eukaryotic expression vectors for expression in different organisms are commercially available. Selection of appropriate expression vectors is within the knowledge of those having skill in the art. The vector may also comprise further elements that aid in the methods of the invention, for example marker genes for selection.

In one embodiment, the vector, for example a binary vector, further may comprise a second expression construct. Use of this vector in the methods of the invention as explained below allows for expression of the selected transcription factor in a cell, tissue or organ in which it is not normally expressed in vivo and/or under conditions under which it is not normally expressed in vivo.

The second expression construct may comprise a first nucleic acid sequence encoding a transcription factor; this is substantially the same sequence as used in the first expression construct. Further, it may comprise a second isolated promoter nucleic acid sequence operably linked to the first nucleic acid sequence encoding a transcription factor. The promoter sequence used in the second construct is distinct from that used in the first construct. However, the transcription factor nucleic acid sequence is substantially the same as the transcription factor nucleic acid sequence used in the first construct. In the organism from which said second promoter sequence is derived, the promoter directs the expression of a gene in a specific cell, tissue or organ in which the transcription factor gene used in the expression construct is not normally expressed and/or the conditions under which the transcription factor gene is not normally expressed in said organism. Therefore, in the second expression construct, the isolated nucleic acid sequence encoding the transcription factor gene is operably linked to a different promoter than in the first construct. In contrast to the first promoter sequence used, the second promoter is not specific to the cell, tissue or organ in which the transcription factor gene is normally expressed and/or the conditions under which the transcription factor gene is normally expressed. Methods using the vector which may comprise the two expression constructs may therefore ensure constitutive expression of a transcription factor gene in a cell, tissue or organ in which the transcription factor gene is not normally expressed.

As explained elsewhere, the transcription factor nucleic acid sequence and the promoter sequence may be of plant, animal or bacterial origins. In a preferred embodiment, the transcription factor nucleic acid sequence and the promoter sequence are of plant origin.

In one embodiment, the promoter of the first construct is specific to root hairs. In a preferred embodiment, the promoter is EXP7 (SEQ ID NO. 1). In one embodiment, the transcription factor gene is RLS4 (SEQ ID No. 2). In one embodiment, the promoter is EXP7 (SEQ ID NO. 1) and the transcription factor gene is RLS4 (SEQ ID No. 2). In one embodiment, the second promoter is GL2. (SEQ ID No. 3). Orthologues and homologues of RSL4 selected from SEQ ID No. 5-117 may also be used.

The first and second expression construct as described herein may be used, either as part of a single vector or by using separate vectors for the expression of the first and second expression construct respectively, in the methods for constitutive expression of a transcription factor in a desired cell, tissue, organ and/or conditions according to the methods of the invention. Transformation of an organism, for example a plant, with such vector(s) allows constitutive expression of the transcription factor in a cell, tissue or organ that normally does not express this transcription factor gene. Thus, once transcription factor expression is initiated from the first expression construct in the desired cell, tissue, organ and/or under the desired conditions, this activates expression of the transcription factor from the second expression construct. Constitutive expression is thus achieved via a positive feedback loop. Accordingly, constitutive expression of genes that encode desirable gene products may thus be achieved in the desired location due to constitutive expression of the transcription factor which in turn activates expression of downstream target genes. For example, if the transcription factor controls the accumulation of secondary metabolites, the use of the two expression constructs as described may both elevate levels of metabolite production and/or target their production to certain cell types.

The present invention also relates to an isolated host cell which may comprise an expression construct or vector of the present invention. In one embodiment, the host cell is a plant cell. For example, a heterologous nucleic acid construct or vector as described herein is introduced into the genome of a plant host cell by transfection, for example with Agrobacterium tumefaciens for plant transformation, microinjection, electroporation, biobalistics or the like.

The invention also relates to a transgenic prokaryotic or eukaryotic organism which has been transformed with the expression construct or vector of the invention and thus expresses the transgene(s). Thus, in one aspect, the invention relates a transgenic organism, for example a plant, which constitutively expresses an endogenous transcription factor gene of interest and wherein said transcription factor is expressed in the same cell, tissue or organ in which it is normally expressed and/or conditions under which it is normally expressed, but at a constitutive level compared to the level of expression in a control organism that does not express the transgene.

In another aspect, the invention relates to a transgenic organism, for example a plant, which constitutively expresses an endogenous transcription factor gene of interest and wherein said transcription factor is expressed in a cell, tissue or organ in which it is not normally expressed and/or conditions under which it is not normally expressed, at a constitutive level compared to the level of expression in a wild type organism that does not express the transgene. As used herein, the terms “transformed”, “stably transformed” or “transgenic” with reference to host organism mean that the transgene is stably integrated within the host genome such that the polynucleotide is passed on to successive generations. Thus, the expression construct(s) and vector(s) described herein may be expressed in a host organism using recombinant DNA technology. Thus, the host organism is transgenic in respect of the expression construct as it may comprise within its genome a heterologous DNA segment. A transgenic plant for the purposes of the invention is thus understood as meaning, as above, that the nucleic acids used in the method of the invention are not at their natural locus in the genome of said plant, it being possible for the nucleic acids to be expressed homologously or heterologously.

A preferred host organism is a plant or part thereof. The term part thereof includes reference to plant organs (for example, leaves, stems, roots, seeds etc.) and plant cells and their progeny and any material that may be harvested from a plant. The term “plant cell”, as used herein includes, without limitation, cells form the following tissues/organs seeds, embryos, meristematic regions, callus tissue, leaves, roots. Also included are gametophytes, sporophytes, pollen, and microspores. Further included are cells in in vitro suspension cultures.

The term “plant” according to the different aspects of the invention includes both monocotyledenous and dicotyledenous plants. In one embodiment, the plant is a dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, capsicum, tobacco, cotton, oilseed rape, okra, apple, rose, strawberry, alfalfa, bean, soybean, field (fava) bean, pea, lentil, peanut, chickpea, apricots, pears, peach, grape vine or citrus species. In one embodiment, the plant is tobacco. In one embodiment, the plant is barley. In one embodiment, the plant is soybean. In one embodiment, the plant is cotton. In one embodiment, the plant is maize (corn). In one embodiment, the plant is rice. In one embodiment, the plant is oilseed rape including canola. In one embodiment, the plant is wheat. In one embodiment, the plant is sugarcane. In one embodiment, the plant is sugar beet.

In one embodiment, the plant is a dicot plant. A monocot plant may, for example, be selected from the families Arecaceae, Amaryllidaceae or Poaceae. For example, the plant may be a cereal crop, such as wheat, rice, barley, maize, oat, sorghum, rye, onion, leek, millet, buckwheat, turf grass, Italian rye grass, switchgrass, Miscanthus, sugarcane or Festuca species.

Preferably, the plant is a crop plant. By crop plant is meant any plant which is grown on a commercial scale for human or animal consumption or use or other non-food/feed use. Non limiting examples of crop plants include soybean, beet, sugar beet, sunflower, oilseed rape including canola, chicory, carrot, cassaya, alfalfa, trefoil, rapeseed, linseed, cotton, tomato, potato, tobacco, poplar, eucalyptus, pine trees, sugarcane and cereals such as rice, maize, wheat, barley, millet, rye, triticale, sorghum, emmer, spelt, secale, einkorn, teff, milo and oats.

Preferred plants are tobacco, maize, wheat, rice, oilseed rape, sorghum, soybean, potato, tomato, barley, pea, bean, cotton, field bean, lettuce, broccoli or other vegetable brassicas or poplar. In another embodiment the plants of the invention and the plants used in the methods of the invention are selected from the group consisting of maize, rice, wheat, soybean, cotton, oilseed rape including canola, sugarcane, sugar beet and alfalfa.

Also included are biofuel and bioenergy crops such as rape/canola, linseed, lupin and willow, poplar, poplar hybrids, switchgrass, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant). In another embodiment, the invention relates to trees, such as poplar or eucalyptus trees.

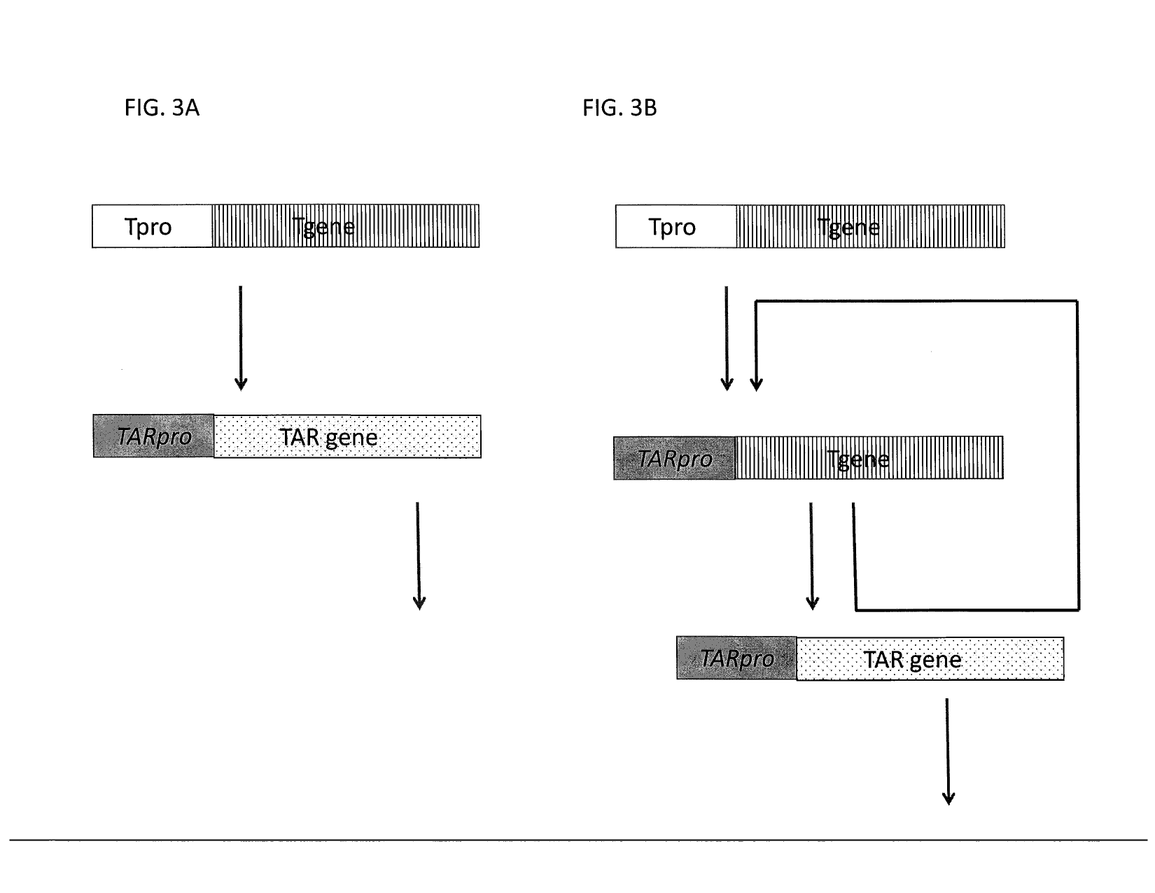

In another aspect, the invention relates to a method for constitutive expression of a transcription factor gene in a host cell or organism. Constitutive expression is compared to expression in a control organism, for example a wild type organism, which does not express the transgene (the expression construct according to the various aspects of the invention). The method may comprise transforming the host cell or organism with an expression construct(s) or vector(s) as described herein which may comprise a nucleic acid sequence encoding a transcription factor operably linked to an isolated promoter nucleic acid sequence of a target gene wherein said transcription factor regulates expression of said target gene. The transgene is stably integrated into the genome of the host cell or organism and thus expressed in the host cell or organism. Preferably, the transcription factor encoding gene is a gene that is normally expressed in a particular cell type, tissue or organ of said organism and/or under specific conditions. Accordingly, in the transformed organism which expresses the transgene, the transcription factor is constitutively expressed in the cell, tissue or organ in which it is normally expressed through a positive feedback loop (see FIG. 3).

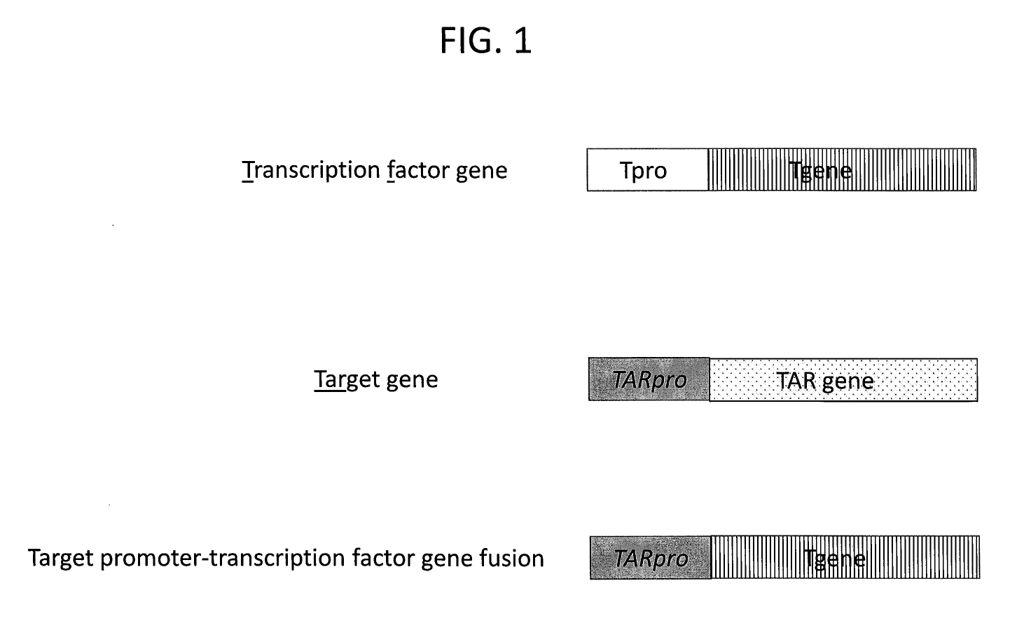

Thus, the transgene or expression construct which is described herein and may comprise a transcription factor encoding gene that is normally expressed in a particular cell type is placed under the control of a promoter of a downstream target gene (see FIG. 1). This construct is then transformed into the host organism. The transcription of the transgene is activated when the endogenous transcription factor gene is expressed and activates transcription of the target promoter. The expression of the transcription factor gene from the transgene in turn activates the expression of the target promoter in the transgene, resulting in still further expression of the transgene. In other words, the transcription factor gene encoded by the construct positively regulates its own transcription. Therefore, once the endogenous transcription factor gene is expressed, this initiates a positive feedback loop that leads to the constitutive expression transcription factor gene from transgene.

The organism may be prokaryotic or eukaryotic as described herein. For example, the organism may be a bacterium, yeast, an animal or preferably a plant. In a preferred embodiment, the organism is a plant. The nucleic acid sequence encoding a transcription factor is a sequence which is endogenous to said organism but which has been operably linked to a promoter sequence that does not usually control expression of the transcription factor gene. Preferably, the invention does not relate to the use of an exogenous nucleic acid sequence encoding a transcription factor. An exogenous sequence is a sequence that does not usually occur in said organism.

In one embodiment, the invention relates to a method for constitutive expression of a plant transcription factor gene in a transgenic plant. The method may comprise transforming a plant with an expression construct or vector as described herein which may comprise a plant transcription factor nucleic acid sequence operably linked to a plant promoter gene sequence wherein said promoter sequence is derived from a plant promoter sequence of a target plant gene of said transcription factor and wherein said transcription factor regulates expression of said target gene. Example 1 shows constitutive expression of the plant transcription factor RSL4 in root hairs using a promoter which drives the expression of the EXP7 gene in plants (EXP7pro-RSL4 construct).

In one embodiment, the transcription factor nucleic acid sequence encodes a transcription factor that is normally expressed in a specific plant tissue or organ and not in the whole plant. In one embodiment, the transcription factor nucleic acid sequence encodes a transcription factor that is normally expressed under specific conditions, such as specific environmental conditions.

Accordingly, because expression of the transcription factor gene is driven by a tissue/organ specific promoter that is the promoter of a downstream target gene of said transcription factor, the transcription factor gene is constitutively expressed in those cells or tissue where it is normally expressed as expression of the transcription factor from the transgene regulates its own expression as the transcription factor encoded by the transgene binds directly or indirectly to the promoter of the transgene to stimulate expression.

In another aspect, the invention relates to a method for constitutive expression of a transcription factor gene in a cell, tissue or organ in which it is not normally expressed. The method may comprise introducing two expression constructs into said organism is as described herein. These may be introduced by using a single vector which may comprise both constructs or by using two vectors. For example, the organism may be transformed with the first vector to generate stable homozygous lines. In a second step, the organism which expresses said first transgene is transformed with the second expression construct, thus generating stable transgenic lines that are homozygous for both transgenes. Alternatively, a first organism may be transformed with the first vector which may comprise a first expression construct to generate stable homozygous lines. A second organism is transformed with the second vector which may comprise a second expression construct to generate stable homozygous lines. Stable homozygous lines derived from the first and second organism are crossed to generate stable homozygous offspring expressing both transgenes.

The first expression construct used in these methods is as described herein and may comprise a nucleic acid sequence encoding a transcription factor operably linked to a promoter sequence wherein said promoter sequence is derived from the promoter sequence of a target gene of said transcription factor and wherein said transcription factor regulates expression of said target gene. The second expression construct may comprise a nucleic acid sequence encoding a transcription factor as in the first construct. However, in the second construct, a nucleic acid sequence encoding a transcription factor is operably linked to a promoter of a gene that is active in desired cell, tissue or organ. As explained above, this leads to a cascade of gene expression in the target tissue.

In one embodiment of this method, the invention relates to a method for constitutive expression of a transcription factor gene in a plant cell, tissue or organ in which it is not normally expressed. Therefore, expression of the transcription factor may be in any plant vegetative or reproductive tissue of interest. In order to achieve stable expression of the transgenic in the plant, a plant may be transformed with both constructs and stable transformants in which the transgenes have been integrated into the genome and are expressed are selected according to methods in the art. Alternatively, a first plant is transformed with the first construct and a second plant is transformed with the second construct. Stable transformants are selected and crossed to achieve co-expression of both constructs. Example 2 shows constitutive expression of GL2:RSL4 and EXP7pro-RSL4 in plants.

Also within the scope of the invention are transgenic cells and organisms obtained or obtainable by the methods of the invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Expression of EXP7pro-RSL4 in Plants

The RSL4 gene controls the expression of the EXP7 gene during root hair development and the promoter of EXP7 is sufficient to drive root hair cell specific gene expression (Keke et al, 2010). We constructed an EXP7:RSL4 gene fusion and transformed Arabidopsis thaliana (EXP7 is the target promoter (TAR) and RSL4 is the upstream transcription factor (T)).

Constructs for Expression of RSL4

A fusion of the EXP7 promoter and the RSL4 coding sequence was made. This is represented here.

### EXP7 Promoter (in Bold)::RSL4 (Underlined)

gagctcgtagttagatgattacaaaggggaaatttaggttaaaagcgtttttttttattctgagtaaaatttgggaatagctttaga
ttgtggggttacagataaagtagagctatgtgttagtaaaagtctttgtggtagtgacttgtgataatatttattgttacaggtaag
tgggaagagagttgggatagttggattggggagcattggatcatttgttgctaaaagacttgaatcatttggctgtgttatctct
tacaactcaaggagtcagaaacagagtagtccataccggtattactctgacattctctcgttagcagagaacaacgatgtact
tgtcctctgctgctctttgacagacgaaacgcaccatattgtgaatagagaagtgatggagttgcttggtaaggatggggttg
tgatcaatgtgggacgaggaaagttgattgatgagaaggagatggtcaagtgtttggttgacggtgtgattggtggtgctgg
tttagatgtgtttgagaatgaaccggcagttcctcaggagttgtttggtttggataatgtagtgttgtctcctcattttgctgtggct
acaccagggtctttggacaatgttgcacagattgctttagctaacttgaaggcgtttttctcgaaccggcctttgctttctccggt
tcaattggattgagagagcgcccggtttgatcaggtagctaaattagttaagctattgtttattataatcaataattcaaaaagaa
agtgtaatgaatatttgaatgtaccctgacattctctcccaaagaagaagaattaatgacgcatattatttaaataattctcccgc
gttgcacatatgactaatttagtcggaacattacgattggcaatataatcataatgtttatgaataaccttttggttctaatgttatt
gtgaaaatactgttaaaacatgatttcatatattagtttatctttggaaacgtaaatagttgacaaacgacaatataaaaataaat
gtctgctgttcaatttaactaatcattgaaaatacataaacgcacgtatatatagacattggatagagtcggtacacgtatcgtc
tatagaacctgctcgcacgtcaacttatactatattcaaaaacctcacttaaacaacaattgaccttttttcctaaattttattagta
tttctattgaaaaaattcaatgaaatgaaacaaatcccaatcggtacggacaaaagtctccaataaaaaaggaattaaaaaaa
aaaaggatagtgatccgcacgtagccaccactactgtcgttgaaaatcccctctatataagattgtctcaaattcgattacttca
tcaaaaaacaaaccaaaaacaaaccctaagaataaagaaaaagaggctagaatgggtccggtaccCATGGACGT
TTTTGTTGATGGTGAATTGGAGTCTCTCTTGGGGATGTTCAACTTTGATCA
ATGTTCATCATCTAAAGAGGAGAGACCGCGAGACGAGTTGCTTGGCCTCT
CTAGCCTTTACAATGGTCATCTTCATCAACATCAACACCATAACAATGTCT
TATCTTCTGATCATCATGCTTTCTTGCTCCCTGATATGTTCCCATTTGGTGC
AATGCCGGGAGGAAATCTTCCGGCCATGCTTGATTCTTGGGATCAAAGTC
ATCACCTCCAAGAAACGTCTTCTCTTAAGAGGAAACTACTTGACGTGGAG
AATCTATGCAAAACTAACTCTAACTGTGACGTCACAAGACAAGAGCTTGC
GAAATCCAAGAAAAAACAGAGGGTAAGCTCGGAAAGCAATACAGTTGAC
GAGAGCAACACTAATTGGGTAGATGGTCAGAGTTTAAGCAACAGTTCAGA
TGATGAGAAAGCTTCGGTCACAAGTGTTAAAGGCAAAACTAGAGCCACC
AAAGGGACAGCCACTGATCCTCAAAGCCTTTATGCTCGGAAACGAAGAG
AGAAGATTAACGAAAGGCTCAAGACACTACAAAACCTTGTGCCAAACGG
GACAAAAGTCGATATAAGCACGATGCTTGAAGAAGCGGTCCATTACGTGA
AGTTCTTGCAGCTTCAGATTAAGTTGTTGAGCTCGGATGATCTATGGATGT
ACGCACCATTGGCTTACAACGGGCCTGGACATGGGGTTCCATCACAACCT
TTTGTCTCGGCTTATGTGAggatcctctagagtcgacctgcaggcatgcaagcttT

This fusion was then ligated into SacI/KpnI-digested pCambia1300 vector (Hajdukiewicz, P et al 1994 The small pPZP family of Agrobacterium binary vectors for plant transformation Plant Molecular Biology 25, 989-994) or any similar vector.

Plant Transformation and Generation of Homozygous Lines Expressing the Transgene

The EXPpro7:RSL4 transgene was transformed into Arabidopsis thaliana plants. Hygromicin-resistant transformants were selected. Self pollinated lines were selected for plants that were either hemizygous or homozygous for the transgene.

Results

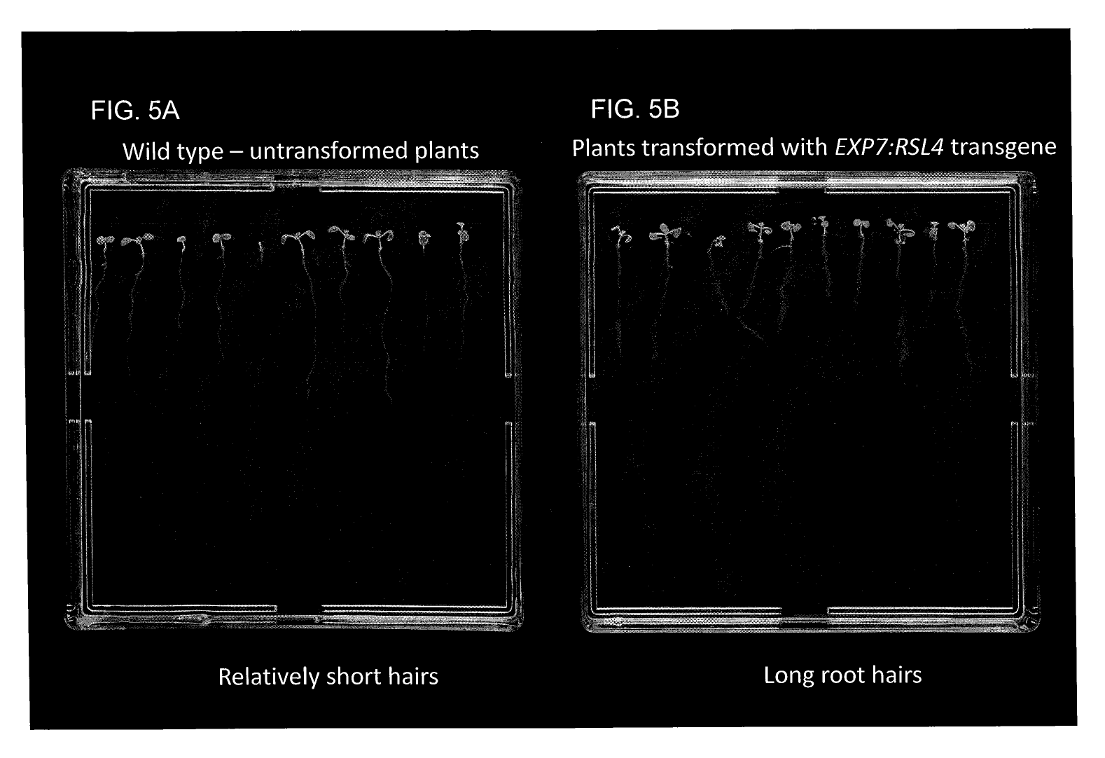

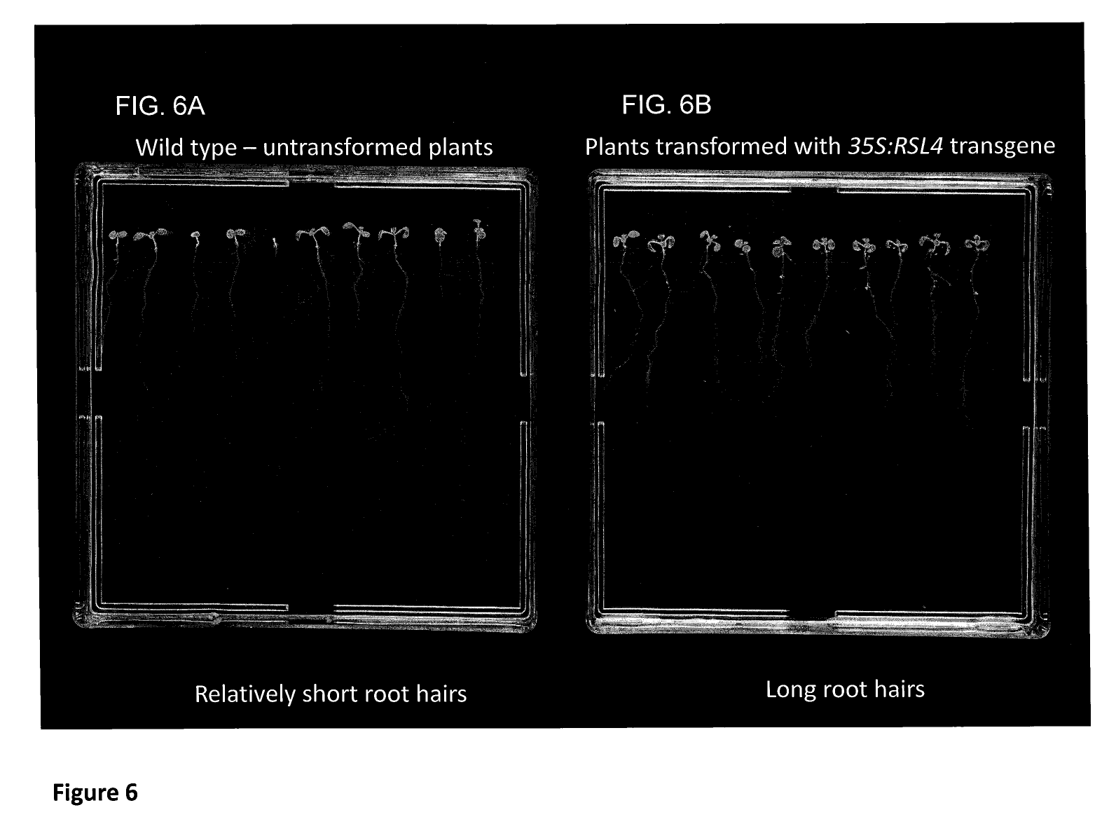

Plants transformed with EXP7:RSL4 had elevated levels of expression of RSL4 transcription indicating that RSL4 is constitutively expressed in root hairs. The root hairs of plants transformed with EXP7-RSL4 grow constitutively until they die and therefore develop very long root hairs (see FIG. 5). This phenotype is identical to that found on roots that constitutively express RSL4 using the CaMV35S promoter (see FIG. 6). Together these data indicates that EXP7:RSL4 results in the constitutive expression of RSL4 in root hair cells.

Without wishing to be bound by theory, we believe that RSL4 positively regulated EXP7 indirectly. That is we think that RSL4 binds to the promoter of another gene that encodes a proteins that in turn binds to the EXP7 promoter.

### Example 2

### Expression GL2:RSL4 and Expression of GL2:RSL4 and EXP7pro-RSL4 in Plants

Constructs for Expression of GL2:RSL4 and Expression of GL2:RSL4 and EXP7pro-RSL4 in Plants

A fusion of the EXP:7 promoter and the RSL4 coding sequence is made. This is represented here.

### EXP7 Promoter (in Bold)::RSL4 (Underlined)

gagctcgtagttagatgattacaaaggggaaatttaggttaaaagcgtttttttttattctgagtaaaatttgggaatagctttaga
ttgtggggttacagataaagtagagctatgtgttagtaaaagtctttgtggtagtgacttgtgataatatttattgttacaggtaag
tgggaagagagttgggatagttggattggggagcattggatcatttgttgctaaaagacttgaatcatttggctgtgttatctct
tacaactcaaggagtcagaaacagagtagtccataccggtattactctgacattctctcgttagcagagaacaacgatgtact
tgtcctctgctgctctttgacagacgaaacgcaccatattgtgaatagagaagtgatggagttgcttggtaaggatggggttg
tgatcaatgtgggacgaggaaagttgattgatgagaaggagatggtcaagtgtttggttgacggtgtgattggtggtgctgg
tttagatgtgtttgagaatgaaccggcagttcctcaggagttgtttggtttggataatgtagtgttgtctcctcattttgctgtggct
acaccagggtctttggacaatgttgcacagattgctttagctaacttgaaggcgtttttctcgaaccggcctttgctttctccggt
tcaattggattgagagagcgcccggtttgatcaggtagctaaattagttaagctattgtttattataatcaataattcaaaaagaa
agtgtaatgaatatttgaatgtaccctgacattctctcccaaagaagaagaattaatgacgcatattatttaaataattctcccgc
gttgcacatatgactaatttagtcggaacattacgattggcaatataatcataatgtttatgaataaccttttggttctaatgttatt
gtgaaaatactgttaaaacatgatttcatatattagtttatctttggaaacgtaaatagttgacaaacgacaatataaaaataaat
gtctgctgttcaatttaactaatcattgaaaatacataaacgcacgtatatatagacattggatagagtcggtacacgtatcgtc
tatagaacctgctcgcacgtcaacttatactatattcaaaaacctcacttaaacaacaattgaccttttttcctaaattttattagta
tttctattgaaaaaattcaatgaaatgaaacaaatcccaatcggtacggacaaaagtctccaataaaaaaggaattaaaaaaa
aaaaggatagtgatccgcacgtagccaccactactgtcgttgaaaatcccctctatataagattgtctcaaattcgattacttca
tcaaaaaacaaaccaaaaacaaaccctaagaataaagaaaaagaggctagaatgggtccggtaccCATGGACGT
TTTTGTTGATGGTGAATTGGAGTCTCTCTTGGGGATGTTCAACTTTGATCA
ATGTTCATCATCTAAAGAGGAGAGACCGCGAGACGAGTTGCTTGGCCTCT
CTAGCCTTTACAATGGTCATCTTCATCAACATCAACACCATAACAATGTCT
TATCTTCTGATCATCATGCTTTCTTGCTCCCTGATATGTTCCCATTTGGTGC
AATGCCGGGAGGAAATCTTCCGGCCATGCTTGATTCTTGGGATCAAAGTC
ATCACCTCCAAGAAACGTCTTCTCTTAAGAGGAAACTACTTGACGTGGAG
AATCTATGCAAAACTAACTCTAACTGTGACGTCACAAGACAAGAGCTTGC
GAAATCCAAGAAAAAACAGAGGGTAAGCTCGGAAAGCAATACAGTTGAC
GAGAGCAACACTAATTGGGTAGATGGTCAGAGTTTAAGCAACAGTTCAGA
TGATGAGAAAGCTTCGGTCACAAGTGTTAAAGGCAAAACTAGAGCCACC
AAAGGGACAGCCACTGATCCTCAAAGCCTTTATGCTCGGAAACGAAGAG
AGAAGATTAACGAAAGGCTCAAGACACTACAAAACCTTGTGCCAAACGG
GACAAAAGTCGATATAAGCACGATGCTTGAAGAAGCGGTCCATTACGTGA
AGTTCTTGCAGCTTCAGATTAAGTTGTTGAGCTCGGATGATCTATGGATGT
ACGCACCATTGGCTTACAACGGGCCTGGACATGGGGTTCCATCACAACCT
TTTGTCTCGGCTTATGTGAggatcctctagagtcgacctgcaggcatgcaagcttT

This fusion is ligated to SacI/KpnI-digested pCambia1300 vector (Hajdukiewicz, P et al 1994 The small pPZP family of Agrobacterium binary vectors for plant transformation Plant Molecular Biology 25, 989-994) or any similar vector.

Plant Transformation and Generation of Homozygous Lines Expressing the Transgene

The EXPpro7:RSL4 transgene is transformed into Arabidopsis thaliana plants. Hygromicin-resistant transformants are selected and grown. Self pollinated lines are selected for plants that are either hemizygous or homozygous for the transgene.

Construction of the gene fusion expressing RSL4 under the control of the GL2 promoter

A fusion of the GL2 promoter and the RSL4 coding sequence is made. This is represented here.

### GL2pro (in Bold)::RSL4 (Underlined)

tctaagcggctttggtctgaattttttatatacaaggcctgtctccgtttttgtaaagggaaaacagtaggatccattttagcctct
gtaagtaacaatattgggcccctaaaagcccacccattttggggccagcaaaccaaggcaccctcggttccgcacgctcg
ctaagacgctaacctatgcatatgttgatatgttttttctcttccttttggtatgaatcttgatttgttttgatactcatgatgtacattc
gtattctcttacgtattgtaaaccatcctatttcagatcacgattatatctttacatttacatttttcatttttatttctgtttgaatgttac
aatttactagtagagttattcattaaaatactacaactggtatacagaaatgtaatttgagtgataaattatatgaaataattaagt
aatatatgtgatatttatggatccaaacaaaaactaattactggttcattttctattttagatgtaagcaaaatgtgtaagattcaag
gtatatatatatcccaatatacgtatatatgtggtactcactagctagtagctctctcacaactgtgtcttttggttttcatcagctg
atcctctccaactaactatccatcttttgttttgcggttggacttggaggtaccaagaatattagcaacgtacgactcgtatggta
tcatttcctttgtacaaaaagtgaatatcaaaatgcattgtattaattatatataagttagtatatggagttagttgtcctcactgtct
ttatctggcgcaatctcctatgccatcattccctcttcacacgtacgtgtgcacactcgatgtcacatttgtataaacacgtttgc
ttttagcgtgagatcatcaccatattccatttttggtgggtcagttctctttctagatagttatttgtaaggacgtgaattaaaagg
gatcgtcgtcacttgttgagataaaagaaaagatatatggtcagtttctgcatcttggaatcaacttaagggttgtcttaattaatt
ttgatagaccctactttaaaaattaattagttgctttcattggccctcaataagaaaagccaaaaaagaaagaagactggtctt
ggaagtttgccaacacgggtaatagattaatggtgaaaagggcgaatttttttacccaaaaccctaattaagtagaagtattaa
tcgagagcaaaaaagagagagagagtcagtagccaaaaggaatgaatggaagaaagaaaaaggaatctctataggcag
catatattcaagtaattaattaaagtagatagatagagcaaaaggagaggttaggaggcattaattaattatttaagagcatgt
ggtgaatgtaaatgtttatggttgcttccctctctatacattatgtatctacctttcctaactaacaattccctaggccgtacgacg
actaacaaagaaaaaaacaaaagaaactgataaagcttttgaattgtagataaatcatctgctacagttataccattatatatct
tattaaagacctaagtttccttcactatacgtcttcgtccatttacgtacgtattatacggacggtttaagctactatatctatattgt
taacaatgtaactgttgagatatatcttgcaataatatgtcatggtgtatgcatacgataatatgaatcaatgtttgaaatcttgac
gtgcccgtgatacaataagatgatcaaaatttcaaattttgtcaaatattaaaacaacatacacatacacatgtgtccaggtgg
cattataaaatgtatatatggtggatatagagagagagggagatgcgtatagtgaataggaaagtaagtaataaagagagg
gtggaggaattggaaaggggttggaggcaaacccataaagagcattcatttccttttaaggtcgctgaaattaatgagtaac
gatcggtcaatgcctctcgctgacctttttctttttttacaacaacaaataaaaataaaataaatttcgacgtctctttccgctgct
gaattacatttgttgaattaattttctctgcttacgtacgtcttctaaactttctctatccgaattcttttttaactttctaacttatattca
acaactcttctttcctgcctttaccgttagtctaattgttttcctaatactgctacgtacatacccctactatactagtcagtgtatta
gattcgattgggattaatccaggaatatagatatcccattagtttttataaaaatattggaagaggacaagtctcaagcaattta
gggttccatgtagcgctgcaatatactgttagtaactctctcttacccatatattgtatatgctaattcttatcaaatatatatatatg
cttctcccagagtcccagtttcctataatcctgacgcaattatactaatagagccaagtttacataataaagtatatatgattaat
agatagggtttcttattaagccatatcttaaattaagatgtgatgatagcgttttgtataagttaccaattgtttgaaagaagagat
catcacaataataaatcataagtagtagtatatagtaataaataaatacacaagtcataataagagtaatgagaggataattaa
ggagggaagaagaaagcagaaaatgcggttggagaattaggtgctaaaagttagttgagtccatctcagtatctaacggtc
aactctctctctctctagagaaaacaattaagaaatctgacatacacatatgtctctctctctctctctctctagtctatacacaca
attcaattaaagaagagacagagaagttcgtcttttttgtttttatacccttaaatcaatcatgcaattgtaacccttccttcttattc
tcattccttccccccctgtctacagtaatctatagcaacgccattatgtactacttttaacggataatttgctcatgtttcaatatgg
cttcattgtatatatgttcaagttcttctcaatcc
GGTACCCATGGACGTTTTTGTTGATGGTGAATTGGAGTCTCTCTTGGGGAT
GTTCAACTTTGATCAATGTTCATCATCTAAAGAGGAGAGACCGCGAGACG
AGTTGCTTGGCCTCTCTAGCCTTTACAATGGTCATCTTCATCAACATCAAC
ACCATAACAATGTCTTATCTTCTGATCATCATGCTTTCTTGCTCCCTGATAT
GTTCCCATTTGGTGCAATGCCGGGAGGAAATCTTCCGGCCATGCTTGATTC
TTGGGATCAAAGTCATCACCTCCAAGAAACGTCTTCTCTTAAGAGGAAAC
TACTTGACGTGGAGAATCTATGCAAAACTAACTCTAACTGTGACGTCACA
AGACAAGAGCTTGCGAAATCCAAGAAAAAACAGAGGGTAAGCTCGGAAA
GCAATACAGTTGACGAGAGCAACACTAATTGGGTAGATGGTCAGAGTTTA
AGCAACAGTTCAGATGATGAGAAAGCTTCGGTCACAAGTGTTAAAGGCA
AAACTAGAGCCACCAAAGGGACAGCCACTGATCCTCAAAGCCTTTATGCT
CGGAAACGAAGAGAGAAGATTAACGAAAGGCTCAAGACACTACAAAACC
TTGTGCCAAACGGGACAAAAGTCGATATAAGCACGATGCTTGAAGAAGC
GGTCCATTACGTGAAGTTCTTGCAGCTTCAGATTAAGTTGTTGAGCTCGGA
TGATCTATGGATGTACGCACCATTGGCTTACAACGGGCCTGGACATGGGG
TTCCATCACAACCTTTTGTCTCGGCTTATGTGAGGATCCTCTAGAGTCGAC
CTGCAGGCATGCAAGCTTT

This fusion is ligated to digested pCambia1300 vector (Hajdukiewicz, P et al 1994 The small pPZP family of Agrobacterium binary vectors for plant transformation Plant Molecular Biology 25, 989-994), or any similar vector.

Plant Transformation and Generation of Homozygous Lines Expressing the Transgene

The GL2:RSL4 transgene is transformed into Arabidopsis thaliana plants. Hygromicin-resistant transformants are selected and grown. Self-pollinated lines are selected for plants that are either hemizygous or homozygous for the transgene. RSL4 is constitutively expressed in these plants.

### REFERENCES

All references cited herein are explicitly incorporated by reference.
Bolduc and Hake, 2009 Plant Cell 21, 1647-1658.
Dubos et al, Trends in Plant Science Vol. 15 No. 10, 573-581
Englebrecht et al, 2004, BMC Genomics 2004, 5:39, http://www.biomedcentral.com/1471-2164/5/39
Gallusci et al 1996, Plant Mol. Biol. 31: 45-50
Heim et al Mol. Biol. Evol. 20(5):735-747. 2003
Hyung-Taeg Cho et al 2004 Plant Cell 14, 3237-3253
Ito, et al., 2006 Plant and Cell Physiology 47, 141-153
Javelle et al 2010 Plant Physiology 154: 273-286
Keke Y, Bell E, Menand B, Dolan L 2010 Nature Genetics 42, 264-267
Inukai et al 2005 Plant Cell 17, 1387
Maddaloni et al. 1996, Mol Gen Genet. 250: 647-654
Morohashi and Grotewold 2010 PLoS Genetics 5(2): e1000396. doi:10.1371/journal.pgen.1000396
Nijhawan et al Plant Physiology Preview. Published on Dec. 7, 2007, as DOI:10.1104/pp. 107.112821
Olsen et al, Trends Plant Sci. 2005 February; 10(2):79-87.
Okushima et al 2007 Plant Cell 19, 118-130
Parenicová et al, Plant Cell 2003 July; 15(7):1538-51.
Pouvreau et al 2011, Plant Cell DOI:10.1104/pp. 111.173641
Riechmann and Ratcliff, 2000, Current Opinion in Plant Biology, 3, 423-434
Riechmann et al, Biol. Chem. 1998 June; 379(6):633-46.
Schlereth et al. 2010 Nature 464, 913-916
Szymanski et al., 1998 Development 125, 1161-1171
Tiwari et al The Plant Cell, Vol. 16, 533-543, February 2004
Yanagisawa Plant Cell Physiol. 45(4): 386-391 (2004)
Yanhui et al Plant Mol. Biol. 2006 January; 60(1):107-24.
Zhang et al 2003 BMC Evolutionary Biology 2005, 5:1
Zhong et al 2007 Plant Cell 19 2776-2792
Zhong et al 2007b Planta 225, 1603-1611

SEQUENCE LISTING

SEQ ID NO 1:
EXP7pro nucleic acid sequence (Hyung-Taeg Cho et al 2004)
GTAGTTAGATGATTACAAAGGGGAAATTTAGGTTAAAAGCGTTTTTTTTTA
TTCTGAGTAAAATTTGGGAATAGCTTTAGATTGTGGGGTTACAGATAAAG
TAGAGCTATGTGTTAGTAAAAGTCTTTGTGGTAGTGACTTGTGATAATATT
TATTGTTACAGGTAAGTGGGAAGAGAGTTGGGATAGTTGGATTGGGGAGC
ATTGGATCATTTGTTGCTAAAAGACTTGAATCATTTGGCTGTGTTATCTCT
TACAACTCAAGGAGTCAGAAACAGAGTAGTCCATACCGGTATTACTCTGA
CATTCTCTCGTTAGCAGAGAACAACGATGTACTTGTCCTCTGCTGCTCTTT
GACAGACGAAACGCACCATATTGTGAATAGAGAAGTGATGGAGTTGCTTG
GTAAGGATGGGGTTGTGATCAATGTGGGACGAGGAAAGTTGATTGATGA
GAAGGAGATGGTCAAGTGTTTGGTTGACGGTGTGATTGGTGGTGCTGGTT
TAGATGTGTTTGAGAATGAACCGGCAGTTCCTCAGGAGTTGTTTGGTTTGG
ATAATGTAGTGTTGTCTCCTCATTTTGCTGTGGCTACACCAGGGTCTTTGG
ACAATGTTGCACAGATTGCTTTAGCTAACTTGAAGGCGTTTTTCTCGAACC
GGCCTTTGCTTTCTCCGGTTCAATTGGATTGAGAGAGCGCCCGGTTTGATC
AGGTAGCTAAATTAGTTAAGCTATTGTTTATTATAATCAATAATTCAAAAA
GAAAGTGTAATGAATATTTGAATGTACCCTGACATTCTCTCCCAAAGAAG
AAGAATTAATGACGCATATTATTTAAATAATTCTCCCGCGTTGCACATATG
ACTAATTTAGTCGGAACATTACGATTGGCAATATAATCATAATGTTTATGA
ATAACCTTTTGGTTCTAATGTTATTGTGAAAATACTGTTAAAACATGATTT
CATATATTAGTTTATCTTTGGAAACGTAAATAGTTGACAAACGACAATAT
AAAAATAAATGTCTGCTGTTCAATTTAACTAATCATTGAAAATACATAAA
CGCACGTATATATAGACATTGGATAGAGTCGGTACACGTATCGTCTATAG
AACCTGCTCGCACGTCAACTTATACTATATTCAAAAACCTCACTTAAACA
ACAATTGACCTTTTTTCCTAAATTTTATTAGTATTTCTATTGAAAAAATTCA
ATGAAATGAAACAAATCCCAATCGGTACGGACAAAAGTCTCCAATAAAA
AAGGAATTAAAAAAAAAAAGGATAGTGATCCGCACGTAGCCACCACTAC
TGTCGTTGAAAATCCCCTCTATATAAGATTGTCTCAAATTCGATTACTTCA
TCAAAAAACAAACCAAAAACAAACCCTAAGAATAAAGAAAAAG
AGGCTAGAATGGGTCC
SEQ ID NO 2:
RSL4 nucleic acid sequence (CDS) (Keke et al 2010)
ATGGACGTTTTTGTTGATGGTGAATTGGAGTCTCTCTTGGGGATGTTCAAC
TTTGATCAATGTTCATCATCTAAAGAGGAGAGACCGCGAGACGAGTTGCT
TGGCCTCTCTAGCCTTTACAATGGTCATCTTCATCAACATCAACACCATAA
CAATGTCTTATCTTCTGATCATCATGCTTTCTTGCTCCCTGATATGTTCCCA
TTTGGTGCAATGCCGGGAGGAAATCTTCCGGCCATGCTTGATTCTTGGGAT
CAAAGTCATCACCTCCAAGAAACGTCTTCTCTTAAGAGGAAACTACTTGA
CGTGGAGAATCTATGCAAAACTAACTCTAACTGTGACGTCACAAGACAAG
AGCTTGCGAAATCCAAGAAAAAACAGAGGGTAAGCTCGGAAAGCAATAC
AGTTGACGAGAGCAACACTAATTGGGTAGATGGTCAGAGTTTAAGCAACA
GTTCAGATGATGAGAAAGCTTCGGTCACAAGTGTTAAAGGCAAAACTAGA
GCCACCAAAGGGACAGCCACTGATCCTCAAAGCCTTTATGCTCGGAAACG
AAGAGAGAAGATTAACGAAAGGCTCAAGACACTACAAAACCTTGTGCCA
AACGGGACAAAAGTCGATATAAGCACGATGCTTGAAGAAGCGGTCCATT
ACGTGAAGTTCTTGCAGCTTCAGATTAAGTTGTTGAGCTCGGATGATCTAT
GGATGTACGCACCATTGGCTTACAACGGGCCTGGACATGGGGTTCCATCA
CAACCTTTTGTCTCGGCTTATGTGA
SEQ ID NO 3;
GL2 nucleic acid sequence of promoter (30033902-30036956) (Szymanski et al., 1998)
tctaagcggctttggtctgaattttttatatacaaggcctgtctccgtttttgtaaagggaaaacagtaggatccattttagcctct
gtaagtaacaatattgggcccctaaaagcccacccattttggggccagcaaaccaaggcaccctcggttccgcacgctcgctaa
gacgctaacctatgcatatgttgatatgttttttctcttccttttggtatgaatcttgatttgttttgatactcatgatgtacattc
gtattctcttacgtattgtaaaccatcctatttcagatcacgattatatctttacatttacatttttcatttttatttctgtttgaatgt
tacaatttactagtagagttattcattaaaatactacaactggtatacagaaatgtaatttgagtgataaattatatgaaataattaagt
aatatatgtgatatttatggatccaaacaaaaactaattactggttcattttctattttagatgtaagcaaaatgtgtaagattcaag
gtatatatatatcccaatatacgtatatatgtggtactcactagctagtagctctctcacaactgtgtcttttggttttcatcagctg
atcctctccaactaactatccatcttttgttttgcggttggacttggaggtaccaagaatattagcaacgtacgactcgtatggta
tcatttcctttgtacaaaaagtgaatatcaaaatgcattgtattaattatatataagttagtatatggagttagttgtcctcactgtct
ttatctggcgcaatctcctatgccatcattccctcttcacacgtacgtgtgcacactcgatgtcacatttgtataaacacgtttgc
ttttagcgtgagatcatcaccatattccatttttggtgggtcagttctctttctagatagttatttgtaaggacgtgaattaaaagg
gatcgtcgtcacttgttgagataaaagaaaagatatatggtcagtttctgcatcttggaatcaacttaagggttgtcttaattaatt
ttgatagaccctactttaaaaattaattagttgctttcattggccctcaataagaaaagccaaaaaagaaagaagactggtctt
ggaagtttgccaacacgggtaatagattaatggtgaaaagggcgaatttttttacccaaaaccctaattaagtagaagtattaa
tcgagagcaaaaaagagagagagagtcagtagccaaaaggaatgaatggaagaaagaaaaaggaatctctataggcag
catatattcaagtaattaattaaagtagatagatagagcaaaaggagaggttaggaggcattaattaattatttaagagcatgt
ggtgaatgtaaatgtttatggttgcttccctctctatacattatgtatctacctttcctaactaacaattccctaggccgtacgacg
actaacaaagaaaaaaacaaaagaaactgataaagcttttgaattgtagataaatcatctgctacagttataccattatatatct
tattaaagacctaagtttccttcactatacgtcttcgtccatttacgtacgtattatacggacggtttaagctactatatctatattgt
taacaatgtaactgttgagatatatcttgcaataatatgtcatggtgtatgcatacgataatatgaatcaatgtttgaaatcttgac
gtgcccgtgatacaataagatgatcaaaatttcaaattttgtcaaatattaaaacaacatacacatacacatgtgtccaggtgg
cattataaaatgtatatatggtggatatagagagagagggagatgcgtatagtgaataggaaagtaagtaataaagagagg
gtggaggaattggaaaggggttggaggcaaacccataaagagcattcatttccttttaaggtcgctgaaattaatgagtaac
gatcggtcaatgcctctcgctgacctttttctttttttacaacaacaaataaaaataaaataaatttcgacgtctctttccgctgct
gaattacatttgttgaattaattttctctgcttacgtacgtcttctaaactttctctatccgaattcttttttaactttctaacttatat
tcaacaactcttctttcctgcctttaccgttagtctaattgttttcctaatactgctacgtacatacccctactatactagtcagtgtat
tagattcgattgggattaatccaggaatatagatatcccattagtttttataaaaatattggaagaggacaagtctcaagcaattta
gggttccatgtagcgctgcaatatactgttagtaactctctcttacccatatattgtatatgctaattcttatcaaatatatatatatg
cttctcccagagtcccagtttcctataatcctgacgcaattatactaatagagccaagtttacataataaagtatatatgattaat
agatagggtttcttattaagccatatcttaaattaagatgtgatgatagcgttttgtataagttaccaattgtttgaaagaagagat
catcacaataataaatcataagtagtagtatatagtaataaataaatacacaagtcataataagagtaatgagaggataattaa
ggagggaagaagaaagcagaaaatgcggttggagaattaggtgctaaaagttagttgagtccatctcagtatctaacggtc
aactctctctctctctagagaaaacaattaagaaatctgacatacacatatgtctctctctctctctctctctagtctatacacaca
attcaattaaagaagagacagagaagttcgtcttttttgtttttatacccttaaatcaatcatgcaattgtaacccttccttcttattc
tcattccttccccccctgtctacagtaatctatagcaacgccattatgtactacttttaacggataatttgctcatgtttcaatatgg
cttcattgtatatatgttcaagttcttctcaatcc
SEQ ID NO. 4
RSL4 peptide sequence
MENEAFVDGELESLLGMFNFDQCSSNESSFCNAPNETDVFSSDDFFPFGTILQ
SNYAAVLDGSNHQTNRNVDSRQDLLKPRKKQKLSSESNLVTEPKTAWRDGQ
SLSSYNSSDDEKALGLVSNTSKSLKRKAKANRGIASDPQSLYARKRRERINDR
LKTLQSLVPNGTKVDISTMLEDAVHYVKFLQLQIKLLSSEDLWMYAPLAHNG
LNMGLHHNLLSRLI
RHD6 amino acid sequence (At1g66470; NP_176820.1 GI: 15219658 SEQ ID NO: 5)
MALVNDHPNETNYLSKQNSSSSEDLSSPGLDQPDAAYAGGGGGGGSASSSST
MNSDHQQHQGFVFYPSGEDHHNSLMDFNGSSFLNFDHHESFPPPAISCGGSS
GGGGFSFLEGNNMSYGFTNWNHQHHMDIISPRSTETPQGQKDWLYSDSTVV
TTGSRNESLSPKSAGNKRSHTGESTQPSKKLSSGVTGKTKPKPTTSPKDPQSL
AAKNRRERISERLKILQELVPNGTKVDLVTMLEKAISYVKFLQVQVKVLATD
EFWPAQGGKAPDISQVKDAIDAILSSSQRDRNSNLITN
RHD6 nucleotide sequence (NM_105318.2 GI: 30697352 SEQ ID NO: 6)
atggcactcgttaatgaccatcccaacgagaccaattacttgtcaaaacaaaattcctcc
tcttccgaagatctctcctcgccgggactggatcagccagatgcagcttatgccggtgga
ggaggaggaggaggctcggcttcgagcagtagcacgatgaattcagatcatcaacaacat
caggggtttgtattttacccatccggtgaagatcatcacaactctttgatggatttcaac
ggatcatcatttcttaactttgatcatcacgagagctttcctcctccagccataagctgt
ggtggtagtagcggtgggggcggcttctccttcttggagggcaacaacatgagctacggc
ttcacaaactggaatcatcaacatcatatggatattattagccctagatccaccgaaact
ccccaaggccagaaagactggttatattctgattcaactgttgtaaccactggttctaga
aacgagtctctttcgcctaaatccgctggaaacaaacgttctcacacgggagagagcact
caaccgtcgaagaaactgagtagcggtgtgaccggaaagaccaagcctaagccaacaact
tcacctaaagatccacaaagcctagcagccaagaatcgaagagaaaggataagtgaacgt
ctcaagatattgcaagaacttgttcccaatggcaccaaggttgatttggtgacaatgctt
gaaaaggctattagttatgtcaagttccttcaagtacaagttaaggtattagcgaccgat
gagttttggccggctcaaggaggaaaagctcctgacatttctcaagttaaagacgccatt
gatgccattctctcctcatcacaacgagacaggaattcgaatctgatcaccaattaa
RSL1 amino acid sequence (At5g37800 SEQ ID NO: 7)
MSLINEHCNERNYISTPNSSEDLSSPQNCGLDEGASASSSSTINSDHQNN
QGFVFYPSGETIEDHNSLMDFNASSFFTFDNHRSLISPVTNGGAFPVVDG
NMSYSYDGWSHHQVDSISPRVIKTPNSFETTSSFGLTSNSMSKPATNHGN
GDWLYSGSTIVNIGSRHESTSPKLAGNKRPFTGENTQLSKKPSSGTNGKI
KPKATTSPKDPQSLAAKNRRERISERLKVLQELVPNGTKVDLVTMLEKAI
GYVKFLQVQV KVLAADEFWP AQGGKAPDIS QVKEAIDAIL
SSSQRDSNSTRETSIAE
RSL1 nucleotide sequence (SEQ ID NO: 8)
atgtcactcattaacgaacattgcaatgagcgtaattacatctcaaccccaaattcttca
gaagatctctcttcaccacagaattgcggattagacgaaggagcttcagcttcaagcagt
agcaccataaattctgatcatcaaaataatcaagggtttgtgttttacccttccggggaa
accattgaagatcataattctttgatggatttcaatgcttcatcattcttcacctttgat
aatcaccgaagccttatctctcccgtgaccaacggtggtgccttcccggtcgtggacggg
aacatgagttacagctatgatggctggagtcatcatcaagtggatagtattagccctaga
gtcatcaaaactccaaatagctttgaaacaacgagcagttttggattgacttcaaactcc
atgagtaaaccggccacaaaccatggaaatggagactggttatactctggttcaactatt
gtaaacatcggttcaaggcacgagtccacgtcccctaaactggctggcaataaacggcct
ttcacgggagagaacacacaactttcaaagaagccgagtagcggtacgaatggaaagatc
aagcctaaggcaacaacttcacctaaagatccacaaagcctagcagccaagaaccgaaga
gaaaggataagcgaacgcctcaaggtattgcaagaacttgtaccgaatggtaccaaggtg
gatttggtaactatgcttgagaaagcaattggctatgtaaagtttcttcaagtacaagtt
aaggtacttgcagccgatgagttttggccggcacaaggagggaaagctccggacatttct
caagttaaagaagctattgacgcaatcctctcatcatcacaacgagatagtaactcaact
agagaaacaagtatagcagaataa
RSL2 amino acid sequence (At4g33880; SEQ ID NO: 9)
MEAMGEWSNN LGGMYTYATE EADFMNQLLA SYDHPGTGSS
SGAAASGDHQ GLYWNLGSHHNHLSLVSEAG SFCFSQESSS YSAGNSGYYT
VVPPTVEENQ NETMDFGMED VTINTNSYLVGEETSECDVE KYSSGKTLMP
LETVVENHDD EESLLQSEIS VTTTKSLTGS KKRSRATSTDKNKRARVNKR
AQKNVEMSGD NNEGEEEEGE TKLKKRKNGA MMSRQNSSTT
FCTEEESNCADQDGGGEDSS SKEDDPSKAL NLNGKTRASR GAATDPQSLY
ARKRRERINE RLRILQNLVP
NGTKVDISTM LEEAVHYVKF LQLQIKLLSS DDLWMYAPIA FNGMDIGLSS
PR
RSL2 nucleotide sequence (SEQ ID NO: 10)
atggaagccatgggagaatggagcaacaacctcggaggaatgtacacttatgcaaccgag
gaagccgatttcatgaaccagcttctcgcctcttatgatcatcctggcaccggctcatcc
tccggcgcagcagccagtggtgaccaccaaggcttgtattggaaccttggttctcatcac
aaccaccttagcctcgtgtctgaagccggtagcttctgtttctctcaagagagcagcagc
tacagcgctgggaacagcggatattacaccgttgttccacccacggttgaagagaaccaa
aatgagacaatggactttgggatggaagatgtgaccatcaatacaaactcataccttgtt
ggtgaggagacaagtgagtgtgacgttgagaaatactcttctggaaagactcttatgcct
ttggaaaccgtagtggagaaccacgatgacgaggaaagcttgttgcaatctgagatctct
gtgactactacaaaatctctcaccggctccaaaaagagatcccgtgccacatctactgat
aaaaacaagagagcaagagtgaataagagggcccagaagaacgtagagatgagtggggat
aacaatgaaggagaagaggaagaaggagagacgaagttgaagaaaagaaagaatggggca
atgatgagtagacagaactcaagcaccactttctgtacggaggaagaatcaaactgcgct
gatcaagacggtggaggagaagactcatcctctaaggaagatgatccctcaaaggccctc
aacctcaatggtaaaacaagagccagtcgtggtgcagccaccgatcctcaaagcctctat
gcaaggaaaagaagagaaaggattaacgagagactaaggattttacaaaatctcgtcccc
aatggaacaaaggtcgatattagtacaatgcttgaggaagcagttcattacgtcaaattt
ttgcagctccaaattaagttattgagctctgatgatctatggatgtatgcgccgattgct
ttcaatgggatggacattggtctcagctcaccgagatga
RSL3 amino acid sequence (At2g14760; SEQ ID NO: 11)
MEAMGEWSTGLGGIYTEEADFMNQLLASYEQPCGGSSSETTATLTAYHHQ
GSQWNGGFCFSQESSSYSGYCAAMPRQEEDNNGMEDATINTNLYLVGEET
SECDATEYSGKSLLPLETVAENHDHSMLQPENSLTTTTDEKMFNQCESSK
KRTRATTTDKNKRANKARRSQKCVEMSGENENSGEEEYTEKAAGKRKTKP
LKPQKTCCSDDESNGGDTFLSKEDGEDSKALNLNGKTRASRGAATDPQSL
YARVDISTML EEAVQYVKFL QLQIKRLLAI GTNHRNRSIP LWTARNRQIS
KAHSRKRLRLRAVAKIIWSDEMTRFLLELITLEKQAGNYRGKS LIEKGKE
NVLVKFKKRFPITLNWNKVNRLDTLKKQYEIYPAKLRSH PLRFIPLLDV
VFRDETVVVE
ESWQPRRGVHRRAPVLDLSDSECPNNNGDEREDLMQNRERDHMRPPTPDW
MSQTPMENSPTSANSDPPFASQERSSTHTQVKNVSRNRKRKQNPADSTLD
RIAATMKKI
RSL3 nucleotide sequence (SEQ ID NO: 12)
atggaagccatgggagaatggagcaccggcctaggcggaatatatacagaggaagctgac
tttatgaatcagctccttgcctcctatgagcaaccttgtggcggttcatcttcagagaca
accgccacactcacggcctaccaccaccagggttctcaatggaatggtggcttttgcttc
tctcaggagagcagtagttatagtggttactgcgcggcgatgccacggcaagaagaagat
aacaatgggatggaggacgcgacaatcaacacgaacttgtaccttgttggtgaagagaca
agtgaatgtgatgcgacggaatactccggtaaaagcctcttgcctttggagactgtcgca
gaaaaccacgaccatagtatgctacagcctgagaactccttgaccacgaccactgatgag
aaaatgttcaaccaatgtgagagttcaaagaagaggacgcgtgccacaacaactgataag
aacaagagagccaacaaggcacgaaggagccagaaatgcgtagagatgagtggcgaaaat
gaaaatagcggcgaagaagaatatacggagaaggctgcggggaagagaaagaccaaacca
cttaagccgcaaaagacttgttgttcggatgacgaatcaaacggtggagacactttcttg
tccaaagaagatggcgaggactctaaggctctcaacctcaacggcaagactagggccagc
cgcggcgcggccacagatcctcaaagcctttacgcaaggaaaagaagagagaggataaac
gagaggctaaggattttgcaacatctcgtccctaatggaacaaaggttgatattagcacg
atgttggaagaagcagtacaatacgtcaaatttctacagctccaaattaagttattgagc
tctgatgatctatggatgtatgcgcctattgcttacaacggaatggacattggccttgac
ctaaaactcaatgcactgaccagatga
RSL5 amino acid sequence (At5g43175; SEQ ID NO: 13)
MENEAFVDGELESLLGMFNFDQCSSNESSFCNAPNETDVFSSDDFFPFGTILQ
SNYAAVLDGSNHQTNRNVDSRQDLLKPRKKQKLSSESNLVTEPKTAWRDGQ
SLSSYNSSDDEKALGLVSNTSKSLKRKAKANRGIASDPQSLYARKRRERINDR
LKTLQSLVPNGTKVDISTMLEDAVHYVKFLQLQIKLLSSEDLWMYAPLAHNG
LNMGLHHNLLSRLI
RSL5 nucleotide sequence (SEQ ID NO: 14)
atggagaatgaagcttttgtagatggtgaattggagtctcttttggggatgttcaacttt
gatcaatgttcatctaacgaatcgagcttttgcaatgctccaaatgagactgatgttttc
tcttctgatgatttcttcccatttggtacaattctgcaaagtaactatgcggccgttctt
gatggttccaaccaccaaacgaaccgaaatgtcgactcaagacaagatctgttgaaacca
aggaagaagcaaaagttaagctcggaaagcaatttggttaccgagcctaagactgcttgg
agagatggtcaaagcctaagcagttataatagttcagatgatgaaaaggctttaggttta
gtgtctaatacatcaaaaagcctaaaacgcaaagcgaaagccaacagagggatagcttcc
gatcctcagagcctatacgctaggaaacgaagagaaaggataaacgataggctaaagaca
ttgcagagcctagttcctaatgggacaaaggtcgatataagcacaatgctggaagatgct
gtccattacgtgaagttcctgcagcttcaaatcaagctcttgagttcagaagatctatgg
atgtatgcacctcttgctcacaatggtctgaatatgggactacatcacaatcttttgtct
cggcttatttaa
AtRHD6 bHLH amino acid sequence (SEQ ID NO: 15)
TSPKDPQSLAAKNRRERISERLKILQELVPNGTKVDLVTMLEKAISYVKFLQV
QVKVLATDEFWPAQ
AtRLD1 bHLH amino acid sequence (SEQ ID NO: 16)
TSPKDPQSLAAKNRRERISERLKVLQELVPNGTKVDLVTMLEKAIGYVKFLQ
VQVKVLAADEFWPAQ
PpRSL1 bHLH amino acid sequence (SEQ ID NO: 17)
GSANDPQSIAARVRRERISERLKVLQALIPNGDKVDMVTMLEKAISYVQCLEF
QIKMLKNDSLWPKA
PpRSL2 bHLH amino acid sequence (SEQ ID NO: 18)
GSANDPQSIAARVRRERISERLKVLQALIPNGDKVDMVTMLEKAITYVQCLE
LQIKMLKNDSIWPKA
PpRSL5 bHLH amino acid sequence (SEQ ID NO: 19)
GSATDPQSVYARHRREKINERLKSLQNLVPNGAKVDIVTMLDEAIHYVKFLQ
NQVELLKSDELWIYA
PpRSL6 bHLH amino acid sequence (SEQ ID NO: 20)
GSATDPQSVYARHRREKINERLKNLQNLVPNGAKVDIVTMLDEAIHYVKFLQ
TQVELLKSDEFWMFA
PpRSL3 bHLH amino acid sequence (SEQ ID NO: 21)
GSATDPQSVYARHRREKINERLKTLQHLVPNGAKVDIVTMLDEAIHYVQFLQ
LQVTLLKSDEYWMYA
PpRSL4 bHLH amino acid sequence (SEQ ID NO: 22)
GSATDPQSVHARARREKIAERLRKLQHLIPNGGKVDIVTMLDEAVHYVQFLK
RQVTLLKSDEYWMYA
PpRSL7 bHLH amino acid sequence (SEQ ID NO: 23)
GSATDPQSVYARHRREKINERLKTLQRLVPNGEQVDIVTMLEEAIHFVKFLEF
QLELLRSDDRWMFA
At4g33880 bHLH amino acid sequence (SEQ ID NO: 24)
GAATDPQSLYARKRRERINERLRILQNLVPNGTKVDISTMLEEAVHYVKFLQ
LQIKLLSSDDLWMYA
At2g14760 bHLH amino acid sequence (SEQ ID NO: 25)
GAATDPQSLYARKRRERINERLRILQHLVPNGTKVDISTMLEEAVQYVKFLQ
LQIKLLSSDDLWMYA
At1g27740 bHLH amino acid sequence (SEQ ID NO: 26)
GTATDPQSLYARKRREKINERLKTLQNLVPNGTKVDISTMLEEAVHYVKFLQ
LQIKLLSSDDLWMYA
At5g43175 bHLH amino acid sequence (SEQ ID NO: 27)
GIASDPQSLYARKRRERINDRLKTLQSLVPNGTKVDISTMLEDAVHYVKFLQ
LQIKLLSSEDLWMYA
Physcomitrella RHD SIX LIKE 1 (PpRSL1) amino acid sequence (SEQ ID NO: 28;
AB084930.1 GI: 140084327)
MAGPAGALWSTCDPQPIQQAEIFSGPDNQAGLMSFHVDTPFHWGSEPWALH
SRSDDIALMSPSLVHDISPYDSVLHLSGVSGDVQDLVCGNPKFRQSGQWGQS
EFSYSVQDNMQDLLTNQFIPYNTSSLGLNHLSPNFTDLDCAPVYNDTKAFGT
VTHNRAVPSTNTQSAQHGSSSMVSSNRPITSTASPTTQYGGPRTPSQTTQYGG
SSMVTNSMEMFASAAPQGIMTTSGLSGGCNSDLMHLPKRQHAHSLPPTTGR
DLTASEVVSGNSISNISGVGSFNSSQKSSASVMMSPLAASSHMHKAAAVSEEL
KMASFNPGPFVPTQKKQQHEQQDTMTSNRIWADKNNLGKISSSPIPIMGFEQS
QQQSMSNSSPVTSLGFEQRQKMSMGSSPSITIIGFEQRQKQPMSSSSPISNMVF
EPRQKQPMSSSSPISNIVFEQRQLPTVGSSPPISISGFEPKKQPSLSNSPPLSNLGF
EQRLQPMSNASPISNLPFEQQRQQATMSNTRSAEPDSVESTTKWPLRMDGAI
GGCAGLPSSQKAPVIMQPETGTMKCPIPRTMPSNAKACPAVQNANSVNKRPL
TVDDKDQTGSMNKKSMQKFLGPQGCSRLESISALAHQKVSQSTTSGRALGP
ALNTNLKPRARQGSANDPQSIAARVRRERISERLKVLQALIPNGDKVDMVTM
LEKAISYVQCLEFQIKMLKNDSLWPKALGPLPNTLQELLELAGPEFAGIDGKN
TEESSEKPKKSALEVIELDGNQPSAD*
Physcomitrella RHD SIX LIKE 1 (PpRSL1) nucleotide sequence (SEQ ID NO: 29;
EF156393.1 GI: 140084326)
atggcaggtccagcaggagctttatggagtacttgtgatccacagcctattcaacaggcagagatatttagtggtcctgaca
accaagctggtttgatgtcttttcatgtggataccccgttccattggggatctgaaccatgggctctccactctcggtcagatg
acatcgccttgatgtccccctcgcttgttcacgacatatcaccttatgattctgtcttgcatctttccggagtgtctggggatgtg
caagatttagtttgcgggaatcccaaatttcgccaaagtgggcaatgggggcagagcgagttttcatactctgttcaggaca
acatgcaagatctcctaaccaaccagttcataccgtacaacacatcttcattgggtttaaatcatctctccccgaatttcaccga
cttggattgcgcaccggtatacaatgataccaaggcttttggcactgttacacacaacagggcagtcccgagcactaatacc
cagagtgctcagcacgggagttcgtctatggtttcaagtaacaggccaatcactagcacagcttctcctactactcagtatgg
aggtccgaggactccatcccaaaccacccagtacgggggttcatctatggttaccaactcgatggaaatgtttgcttcagct
gcacctcagggtattatgactacatctggcttgagtggcggttgcaactcagacttgatgcatctgccgaagcgccagcatg
ctcactctcttcctcctaccactggcagagatttaactgcatctgaagtggtatctggaaattcgatatcaaacatttccggggt
tggatcttttaacagcagccagaaaagcagtgcatccgtgatgatgtctcctttagctgcttcttctcacatgcacaaggctgc
tgctgtatctgaagaacttaagatggcaagtttcaaccctggtccattcgtacctacgcagaaaaagcagcaacatgagcag
caggatacgatgacctctaatcgtatatgggcggataagaacaacttgggaaaaattagttcatcgcccattccgatcatgg
ggtttgagcagagtcaacagcaatccatgagcaattcctcccctgttaccagtttggggtttgagcaaaggcaaaaaatgtc
catgggtagctctccctccatcacgatcattggatttgagcaaagacagaagcaacctatgagtagttcttcccccatttcaaa
catggtttttgaaccaagacaaaaacagccaatgagtagctcttctcctatctctaatattgtctttgagcaaagacaactccca
actgtgggtagctctcctccgatttcaatctcaggatttgagccaaagaaacaaccatctttgagcaattctcctcccctctcta
atctgggttttgagcaaaggctacaacccatgagtaatgcatctcctatttccaacttaccctttgagcaacaaagacaacaa
gcaaccatgagtaacaccagatctgcagaacccgattctgtcgagtctaccacgaagtggcccttgcggatggatggtgc
cataggtggatgtgctggcttaccaagcagtcagaaagctcctgttatcatgcagcctgagactgggactatgaagtgtcct
attccgaggaccatgcccagcaatgctaaggcttgcccagctgtgcagaatgctaattccgtaaacaagcgccctcttacg
gttgatgacaaggaccaaactggatcgatgaataagaagtcgatgcaaaagtttttgggacctcaaggttgtagcagacttg
aaagtatcagtgctttagctcaccaaaaagtgagtcaaagtacaacaagcggtcgtgctctagggcctgctttgaacaccaa
tctcaagcctcgtgcacgccaagggagtgccaatgatccgcagagcattgctgctagggtgcgaagagaaagaataagt
gagcggctcaaagttttgcaagccttgatacctaacggtgataaagtggatatggtcaccatgctggagaaggctatcagct
acgtgcagtgtttggaatttcagattaagatgttaaaaaatgactctttgtggcctaaggcgcttggccctctaccgaacacttt
gcaagagcttctcgaacttgctgggccagagtttgccggcatagatggcaagaatactgaggagtcgtcagagaaaccga
agaaatctgctcttgaagtaattgagttggacggcaatcagccttctgctgactaa
Physcomitrella RHD SIX LIKE 2 (PpRSL2) amino acid sequence (SEQ ID NO: 30
AB084931.1 GI: 140084334)
MNKKPMQKALGPQGCSRLESISALAHQKVSQSASGRALGPALNTNLKPRAR
QGSANDPQSIAARVRRERISERLKVLQALIPNGDKVDMVTMLEKAITYVQCL
ELQIKMLKNDSIWPKALGPLPNTLQELLELAGPEFSGTESKNVEEPPAKPKKS
APDVIEFDGNQPSADKE*
Physcomitrella RHD SIX LIKE 2 (PpRSL2) nucleotide sequence (SEQ ID NO: 31;
EF156394.1 GI: 140084333)
atgaataagaagcctatgcaaaaagctttgggacctcaaggatgcagcaggctagaaagcatcagtgctttagctcatcaa
aaagtgagtcagagtgcaagtggtcgtgcactagggcctgctctgaacaccaacctcaagcctcgtgctcgtcaagggag
tgccaatgacccacagagcattgccgctagggttcgaagagaaaggataagtgagcggctgaaagttttgcaagccttgat
acctaatggtgataaggtagatatggtgaccatgctggagaaggctatcacctacgtgcagtgtctggaactccagattaag
atgttaaagaatgattctatctggcccaaggcgcttggacctctaccaaacactcttcaagagcttctggagcttgctggacc
agaattttctggaacggaaagcaagaatgtagaggagcccccagcgaagccaaagaaatcagctcctgacgttattgagtt
cgacggcaatcaaccttctgccgacaaagagtag
Physcomitrella RHD SIX LIKE 3 (PpRSL3) amino acid sequence (SEQ ID NO: 32;
AB084932.1 GI: 140084346)
GSATDPQSVYARHRREKINERLKTLQHLVPNGAKVDIVTMLDEAIHYVQFLQ
LQVTLLKSDEYWMYA
Physcomitrella RHD SIX LIKE 3 (PpRSL3) nucleotide sequence (SEQ ID NO: 33;
EF156395.1 GI: 140084345)
ggttcagcgactgatccgcagagtgtatatgccaggcatagaagggagaagatcaacgagcgcttgaagacattacagc
acttggtaccaaatggagctaaggtagacatcgtgaccatgcttgacgaagccattcactacgtccaatttctgcagctccaa
gtgacgctgttgaagtcggatgaatattggatgtacgcc
Physcomitrella RHD SIX LIKE 4 (PpRSL4) amino acid sequence (SEQ ID NO: 34;
AB084933.1 GI:140084359)
MTDLISILESSGSSREEMCPVAVPSSVASSCERLIWEGWTAQPSPVEESTTSKL
LPKLLPELETSSYSALTLQQPDALSSILSVLHPFSHYSSASLELARNPDWSLKSS
NPLRESSSEAGIRTSSFEGLYSGQHTTKKIHLGVIPYHLSEDQRQCAVSPPENE
CRLLSANSSGSLHWWHSIGPESPSSTLAFHNIGIQHSTFEKCEPRGQSHSSWPA
ASGTSPTVQYFHAHSADNEGVEVVKQDDSQISKALATYQPHGDHSLVLNSD
RIASTTSHSEDPCGPKPGRRPAASYDTEMILSPSESFLTTPNMLSTLECVISGAS
NISDQYMNFVREPQEQRLSSISDLSLIPDSHADPHSIGFISGTFRTDSHGTGIRK
NRIFLSDEESDFLPKKRSKYTVRGDFQMDRFDAVWGNTGLRGSSCPGNSVSQ
MMAIYEFGPALNRNGRPRVQRGSATDPQSVHARARREKIAERLRKLQHLIPN
GGKVDIVTMLDEAVHYVQFLKRQVTLLKSDEYWMYATPTSYRSKFDDCSLV
PGENN*
Physcomitrella RHD SIX LIKE 4 (PpRSL4) nucleotide sequence (SEQ ID NO: 35;
EF156396.1 GI: 140084358)
ATGACCGATCTGATTTCGATCTTGGAGTCATCAGGGTCATCACGAGAGGA
GATGTGCCCTGTTGCTGTGCCAAGCTCCGTGGCTTCTTCTTGTGAAAGGTT
GATATGGGAGGGGTGGACTGCACAACCATCTCCTGTCGAAGAAAGCACC
ACCAGCAAGTTACTTCCAAAGCTACTTCCAGAGCTCGAGACATCATCCTA
CTCTGCACTCACCCTTCAGCAACCTGATGCGCTCTCCAGCATACTTTCAGT
CCTCCACCCTTTTTCTCATTACAGTTCGGCCAGTTTAGAACTCGCTCGCAA
TCCTGACTGGAGCTTGAAATCTTCAAATCCTCTGCGGGAAAGCAGCTCGG
AGGCTGGCATCCGAACCTCATCTTTCGAAGGCTTGTACTCTGGTCAGCAC
ACCACCAAAAAGATTCATTTGGGGGTCATACCCTACCACTTGTCCGAAGA
TCAGCGCCAGTGCGCTGTCAGTCCTCCGGAAAATGAGTGCCGCCTACTGT
CTGCAAATTCCTCTGGATCCCTTCACTGGTGGCATTCCATAGGCCCCGAGT
CTCCTTCCTCTACTCTTGCATTCCATAATATTGGGATCCAACACTCTACCTT
CGAAAAGTGTGAGCCTAGGGGCCAGTCGCACTCATCATGGCCAGCGGCC
AGCGGCACGTCGCCAACAGTTCAATACTTTCATGCCCATTCTGCAGATAA
TGAAGGTGTCGAGGTCGTCAAGCAAGATGACTCGCAGATATCCAAGGCTC
TGGCGACCTATCAACCCCACGGCGACCATAGTCTCGTGCTAAATTCAGAC
CGCATTGCAAGCACAACCAGCCACTCAGAAGATCCTTGCGGCCCTAAACC
TGGACGCAGACCAGCTGCATCATACGACACCGAGATGATTCTTAGCCCAA
GTGAGAGTTTCTTGACAACTCCCAATATGTTATCAACGTTGGAGTGCGTA
ATATCCGGTGCAAGTAACATATCTGATCAGTATATGAACTTCGTCAGAGA
ACCGCAGGAGCAAAGGCTGTCCTCTATCTCCGATCTGTCCCTTATTCCTGA
CAGCCACGCGGATCCGCACAGTATCGGATTTATCTCTGGGACCTTTAGAA
CAGACTCCCACGGAACTGGAATAAGAAAGAACCGCATCTTTCTCAGTGAT
GAGGAATCCGACTTCTTGCCTAAGAAGCGATCCAAGTACACGGTCCGCGG
CGATTTTCAGATGGATCGCTTCGACGCAGTTTGGGGGAATACCGGTCTTC
GGGGATCTAGCTGTCCTGGAAATTCAGTATCCCAGATGATGGCGATTTAC
GAATTCGGACCCGCACTGAACAGGAACGGCAGGCCGCGAGTACAACGTG
GTTCGGCGACTGATCCGCAGAGTGTACACGCCAGGGCGCGGAGGGAGAA
AATCGCCGAGCGCTTGAGAAAGTTGCAGCACCTCATTCCAAACGGCGGGA
AGGTGGACATCGTAACCATGCTCGACGAAGCCGTTCACTATGTTCAGTTTT
TGAAGCGACAAGTTACGCTTCTGAAATCCGACGAGTATTGGATGTACGCC
ACGCCGACCTCGTACCGGAGCAAATTCGACGACTGCAGTCTGGTTCCCGG
CGAGAACAACTGA
Physcomitrella RHD SIX LIKE 5 (PpRSL5) amino acid sequence (SEQ ID NO: 36:
AB084934.1 GI: 140084368)
MVQLYMSSVEEQRETMVQPYVSSMDSGSTSGRQTPSCVVQQGSNTFETSNL
WEEWTQASNGDDTVSTSNFLPEISSFTSSRLSFQQSDSLTTWMSGFPPLSQTA
LSPDLSHSSDPVDHPPAFMQEGLGPGDSILDYSPALTEMYPKSSSKHNSSDCL
PYPAASAPDKKMTDHELGSAISLAYDRGTVSRQLLRALGPLSPSSPLALQNGL
QNPLGDPWDASPSAMPWPMATTGHAYGPGATRTSIPDHLANAINHLEGIAPS
SASHASKPRHTDIFIAPNGTFDSTPGGWTPQYYDGSVTTDESVKAMKLIASLR
EAGHAEATIGFCTESKPSFLRGGDRTTSPVDSFFGKCVGAKTSIKQACSGKHP
LELEEIVDSENSELNPTQLKRSKLFENHPNALWSDQSMNGRELRSYSHLVGSS
LTASQPMDIIAIGPALNTDGKPRAKRGSATDPQSVYARHRREKINERLKSLQN
LVPNGAKVDIVTMLDEAIHYVKFLQNQVELLKSDELWIYATPNKYNGMDISD
LSDMYLQELESRA*
Physcomitrella RHD SIX LIKE 5 (PpRSL5) nucleotide sequence (SEQ ID NO: 37;
EF156397.1 GI: 140084367)
ATGGTGCAGTTATACATGTCCTCAGTTGAAGAGCAGCGGGAAACAATGGT
ACAGCCATACGTCTCAAGCATGGACTCAGGCTCAACGTCGGGGCGCCAGA
CGCCATCTTGCGTCGTTCAGCAGGGAAGTAACACATTTGAGACTTCGAAT
CTGTGGGAGGAATGGACGCAAGCATCGAACGGCGACGATACAGTCTCCA
CCAGCAATTTCCTCCCCGAAATCAGTTCCTTCACGTCGAGTCGTCTCTCCT
TCCAGCAAAGCGACTCTCTCACCACTTGGATGTCAGGGTTCCCTCCCCTCT
CCCAAACTGCCTTGAGCCCGGATCTTAGTCACTCCTCCGACCCCGTGGATC
ATCCCCCAGCATTCATGCAGGAGGGTTTAGGCCCCGGTGATTCTATTCTGG
ACTATTCCCCCGCTCTCACAGAGATGTACCCGAAAAGTAGCTCCAAACAT
AATTCCTCGGATTGTTTACCTTACCCTGCGGCCAGTGCACCAGACAAAAA
AATGACTGATCACGAACTAGGTTCGGCTATTTCCCTCGCGTATGATAGAG
GCACCGTTTCCCGCCAGCTTCTTCGAGCCTTGGGCCCATTGTCGCCTTCAT
CGCCTCTAGCATTGCAGAATGGGCTGCAAAACCCGCTTGGGGACCCCTGG
GATGCTTCTCCATCTGCAATGCCGTGGCCAATGGCAACAACCGGTCATGC
TTATGGACCAGGCGCCACCAGGACTTCTATTCCAGATCACTTAGCAAATG
CAATTAATCACCTGGAGGGCATTGCACCGTCCAGTGCCAGTCATGCATCG
AAACCTCGTCACACTGATATTTTCATTGCACCCAATGGCACGTTCGATTCG
ACGCCGGGAGGTTGGACACCGCAGTATTACGATGGGTCCGTGACGACAG
ATGAGTCTGTGAAGGCGATGAAGCTGATTGCGTCCCTACGTGAAGCAGGC
CACGCAGAGGCTACAATTGGATTCTGTACAGAGAGCAAGCCTAGTTTTCT
CAGGGGTGGGGACAGAACAACCTCGCCAGTGGACAGCTTCTTCGGCAAA
TGTGTAGGGGCCAAAACGAGTATAAAGCAAGCCTGTTCTGGGAAACACCC
TCTTGAACTTGAGGAGATCGTTGATAGTGAAAACAGTGAATTAAATCCCA
CCCAGCTCAAACGCTCTAAACTTTTTGAGAATCATCCGAATGCCTTGTGGA
GCGATCAGAGTATGAATGGAAGAGAACTGAGATCGTACTCTCATTTGGTT
GGCAGCAGTCTTACTGCATCGCAGCCCATGGACATAATTGCAATTGGCCC
AGCGCTCAACACTGATGGCAAACCACGAGCAAAGCGGGGTTCAGCAACC
GATCCTCAGAGTGTTTACGCTAGACATAGGAGAGAAAAAATCAACGAAC
GATTGAAGAGTTTACAAAACCTAGTACCTAATGGAGCCAAGGTTGACATA
GTAACCATGCTGGACGAAGCTATACATTACGTCAAATTTTTACAAAATCA
AGTTGAGCTGCTGAAGTCCGACGAGTTGTGGATTTACGCAACACCAAATA
AGTACAACGGCATGGACATTTCCGACCTCTCTGACATGTATTTGCAGGAG
CTGGAGTCACGTGCGTGA
Physcomitrella RHD SIX LIKE 6 (PpRSL6) amino acid sequence (SEQ ID NO: 38;
AB084935.1 GI: 140084376)
MVRFNYMYPVQEQLEAMTDQHTPSMDSVSSAGEKTSSCIVQQGGNASETSN
LWEEWTQGSNGDDSVSTSNFLPELNSSTSSRLAFHQSDILSTWISGYHPLSQSS
LSSEFSHTSDRENHPPAFMQEGLIPSGLILDSDPALTDIYTRSSSSDSLPYPTARI
MDKALTDHELESAVPLAYEKGCVPPQVLRNLGPLSPSSPLAFQNGLLNPLRD
PWDSCPSALPWSNVTTASQTYGQVTTRTFIPDHSASAIDKLEAVATITAGYGA
SKPQHTDVFIEPNGTFQSTPAGWAPQFYDGSEATGLLVKPMRAIASLGEAGC
GEATSEFCTKTKPGLLKGGDTITSPVGSLLGDCKKAESSMKQVWPGKHRLEL
VELVDGEDTKSSPTQLKRPKHSTDYANVLLSDHILKGAELRSYFHSGDVGLN
ASQAMDIIVIGPALNTNGKPRAKRGSATDPQSVYARHRREKINERLKNLQNL
VPNGAKVDIVTMLDEAIHYVKFLQTQVELLKSDEFWMFANPHNYNGIDISDP
SSMHSPELESNI*
Physcomitrella RHD SIX LIKE 6 (PpRSL6) nucleotide sequence (SEQ ID NO: 39:
EF156398.1 GI: 140084375)
ATGGTGCGGTTTAACTACATGTACCCGGTTCAAGAGCAGCTGGAAGCCAT
GACGGACCAACACACCCCAAGCATGGATTCGGTCTCGTCGGCCGGAGAG
AAGACATCCTCTTGCATCGTCCAGCAGGGAGGAAATGCATCCGAAACTTC
AAACTTGTGGGAAGAATGGACACAAGGGTCGAACGGCGACGATTCTGTCT
CTACCAGCAACTTCCTCCCCGAACTGAATTCCTCCACCTCCAGTCGTCTCG
CATTCCACCAAAGCGACATTCTTTCCACTTGGATCTCAGGCTACCACCCAC
TCTCGCAAAGCAGCCTGAGTTCCGAATTCAGCCACACCTCCGACCGCGAG
AATCACCCCCCAGCATTCATGCAAGAGGGTTTAATCCCCAGTGGTTTAATT
CTTGACTCTGATCCTGCTCTCACAGATATTTATACGAGAAGCAGCTCCTCG
GACTCTTTGCCATACCCCACGGCTAGGATCATGGACAAAGCATTGACCGA
TCACGAGCTTGAGTCTGCTGTCCCACTTGCATATGAAAAAGGCTGCGTTCC
TCCCCAGGTTCTGCGTAACCTAGGGCCATTGTCACCTTCTTCGCCTCTGGC
ATTCCAGAATGGACTGCTAAACCCCCTCAGGGACCCTTGGGATTCGTGTC
CATCTGCATTGCCATGGTCAAATGTGACCACAGCCAGCCAGACTTACGGT
CAAGTGACAACCAGGACTTTCATTCCAGATCACTCTGCAAGTGCAATCGA
CAAGTTGGAGGCCGTCGCAACGATCACTGCCGGATACGGCGCGTCGAAA
CCACAACATACTGACGTCTTCATAGAACCCAACGGGACGTTTCAGTCGAC
TCCGGCAGGGTGGGCACCGCAGTTTTACGATGGATCCGAGGCGACGGGCC
TGTTGGTCAAGCCAATGAGGGCCATCGCATCTCTGGGTGAAGCCGGCTGT
GGGGAGGCCACTAGTGAATTCTGCACAAAGACCAAGCCAGGACTTCTCA
AAGGTGGGGACACAATAACCTCGCCGGTGGGTAGCCTGTTGGGCGATTGC
AAAAAAGCTGAGTCAAGTATGAAGCAAGTTTGGCCTGGAAAACACCGTCT
TGAACTCGTGGAACTAGTCGATGGTGAAGACACCAAATCAAGTCCCACCC
AGCTCAAACGGCCGAAACATTCTACGGATTATGCGAATGTCCTGTTGAGC
GATCATATTCTGAAAGGAGCGGAGCTGCGGTCCTACTTCCATTCTGGTGA
TGTTGGTCTAAATGCATCTCAAGCGATGGACATTATTGTAATTGGCCCAGC
CTTGAATACTAATGGCAAGCCGCGAGCTAAACGGGGTTCAGCCACCGATC
CCCAGAGTGTGTACGCTAGACATAGGCGAGAAAAAATCAACGAACGACT
GAAGAATTTACAAAATCTCGTGCCAAATGGAGCCAAGGTTGACATTGTGA
CCATGCTAGACGAAGCCATACACTACGTCAAATTCTTGCAAACTCAAGTT
GAGCTGCTGAAATCCGACGAGTTCTGGATGTTCGCAAATCCACACAACTA
CAACGGCATAGATATCTCCGATCCCTCTAGCATGCATTCGCCGGAGCTGG
AGTCGAATATTTAG
Physcomitrella RHD SIX LIKE 7 (PpRSL7) amino acid sequence (SEQ ID NO: 40;
ABO84936.1 GI: 140084384)
GSATDPQSVYARHRREKINERLKTLQRLVPNGEQVDIVTMLEEAIHFVKFLEF
QLELLRSDDRWMFA
Physcomitrella RHD SIX LIKE 7 (PpRSL7) nucleotide sequence (SEQ ID NO: 41;
EF156399.1 GI: 140084383)
Gggtcagctactgatcctcagagtgtgtacgcaaggcatcgccgggagaagattaacgagcgcctaaagacattgcagc
ggttggttcctaacggagaacaggtcgacattgtgaccatgctggaagaagccattcactttgtcaaatttttggagttccaac
tggagctgttgcgatccgatgatcgctggatgttcgcc
Selaginella moelendorfii SmRSLa amino acid sequence (SEQ ID NO: 42)
LNTNLKPRAKQGCANDPQSIAARQRRERISDRLKILQELIPNGSKVDLVTMLE
KAINYVKFLQLQVKVLMNDEYWPPKGD
Selaginella moelendorfii SmRSLa nucleotide sequence(SEQ ID NO: 43)
CTCAACACTAATCTTAAGCCGCGAGCAAAGCAAGGTTGTGCTAATGATCC
ACAAAGCATTGCTGCCAGACAACGAAGAGAACGGATAAGTGACCGGCTT
AAAATCCTGCAGGAGCTCATACCAAATGGATCCAAGGTCGATCTGGTAAC
CATGCTGGAGAAGGCCATCAACTACGTCAAGTTCTTGCAATTGCAAGTCA
AAGTTCTTATGAACGATGAGTATTGGCCACCAAAGGGAGAT
Selaginella moelendorfii SmRSLb amino acid sequence (SEQ ID NO: 44)
LNTNLKPRAKQGCANDPQSIAARQRRERISDRLKILQELIPNGSKVDLVTMLE
KAINYVKFLQLQVKVLMNDEYWPPKGD
Selaginella moelendorfii SmRSLb nucleotide sequence (SEQ ID NO: 45)
CTCAACACTAATCTTAAGCCGCGAGCAAAGCAAGGTTGTGCTAATGATCC
ACAAAGCATTGCTGCCAGACAACGAAGAGAACGGATAAGTGACCGGCTT
AAAATCCTGCAGGAGCTCATACCAAATGGATCCAAGGTCGATCTGGTAAC
CATGTTGGAGAAGGCCATCAACTACGTCAAGTTCTTGCAATTGCAAGTCA
AAGTTCTTATGAACGATGAGTATTGGCCACCAAAGGGAGAT
Selaginella moelendorfii SmRSLc amino acid sequence (SEQ ID NO: 46)
LNTNFKPRARQGSANDPQSIAARHRRERISDRLKILQELVPNSTKVDLVTMLE
KAINYVKFLQLQVKVLTSDDYWP
Selaginella moelendorfii SmRSLc nucleotide sequence (SEQ ID NO: 47)
CTCAACACCAATTTCAAGCCTCGAGCCAGGCAGGGAAGCGCCAATGATCC
CCAGAGCATCGCTGCTAGACATCGCCGGGAGAGGATCAGTGACAGGCTC
AAGATCTTGCAAGAGCTCGTTCCAAACAGCACAAAGGTTGATCTAGTGAC
GATGCTGGAGAAGGCCATCAATTACGTCAAGTTCCTCCAGCTGCAAGTTA
AGGTGCTTACGTCGGACGACTACTGGCCA
Selaginella moelendorfii SmRSLd amino acid sequence (SEQ ID NO: 48)
LNTNFKPRARQGSANDPQSIAARHRRERISDRLKILQELVPNSTKVDLVTMLE
KAINYVKFLQLQVKVLTSDDYWP
Selaginella moelendorfii SmRSLd nucleotide sequence (SEQ ID NO: 49)
CTCAACACCAATTTCAAGCCTCGAGCCAGGCAGGGAAGCGCCAATGATCC
CCAGAGCATCGCTGCTAGACATCGCCGGGAGAGGATCAGTGACAGGCTC
AAGATCTTGCAAGAGCTCGTTCCAAACAGCACAAAGGTTGATCTAGTGAC
GATGCTGGAGAAGGCCATCAATTACGTCAAGTTCCTCCAGCTGCAAGTTA
AGGTGCTTACGTCGGACGACTATTGGCCA
Selaginella moelendorfii SmRSLe amino acid sequence (SEQ ID NO: 50)
LNTDGKPRAKRGSATDPQSIYARQRRERINERLRALQGLVPNGAKVDIVTML
EEAINYVKFLQLQVKLLSSDEYWMYAPT
Selaginella moelendorfii SmRSLe nucleotide sequence (SEQ ID NO: 51)
CTAAACACCGACGGAAAGCCACGCGCAAAGCGTGGATCTGCCACGGACC
CGCAAAGCATCTACGCTCGGCAAAGAAGAGAAAGGATCAACGAGCGTTT
GAGAGCGCTACAAGGACTCGTACCAAACGGAGCGAAGGTTGACATTGTG
ACGATGCTCGAGGAAGCCATCAACTATGTCAAGTTTTTGCAGCTGCAAGT
AAAGCTGCTCAGCTCGGACGAGTATTGGATGTACGCCCCCACA
Selaginella moelendorfii SmRSLf amino acid sequence (SEQ ID NO: 52)
LNTNGKPRAKRGSATDPQSVYARHRRERINERLKTLQHLVPNGAKVDIVTM
LEEAIHYVKFLQLQVNMLSSDEYWIYAPT
Selaginella moelendorfii SmRSLf nucleotide sequence (SEQ ID NO: 53)
CTCAACACGAATGGCAAGCCCAGAGCAAAGCGTGGATCTGCAACAGATC
CCCAAAGCGTTTACGCAAGGCACCGGAGAGAGAGGATCAACGAGAGGCT
CAAAACTTTACAACACCTTGTTCCAAATGGTGCAAAGGTTGACATAGTGA
CAATGCTTGAAGAAGCAATACATTACGTGAAGTTTCTACAGCTGCAAGTC
AACATGTTAAGCTCTGATGAGTACTGGATTTATGCACCCACA
Selaginella moelendorfii SmRSLg amino acid sequence (SEQ ID NO: 54)
LNTNGKPRAKRGSATDPQSVYARHRRERINERLKTLQHLVPNGAKVDIVTM
LEEAIHYVKFLQLQVNMLSSDEYWTYAPT
Selaginella moelendorfii SmRSLg nucleotide sequence (SEQ ID NO: 55)
CTCAACACGAATGGCAAGCCCCGAGCAAAGCGTGGATCTGCAACAGATC
CCCAAAGCGTTTATGCAAGGCACCGGAGAGAGAGGATCAACGAGAGGCT
CAAAACTTTACAACACCTTGTTCCAAATGGTGCAAAGGTTGACATTGTGA
CAATGCTTGAAGAAGCAATACATTACGTGAAGTTTCTACAGCTGCAAGTC
AACATGTTAAGCTCTGATGAGTACTGGACTTATGCACCCACA
Selaginella moelendorfii SmRSLh amino acid sequence (SEQ ID NO: 56)
LNTDGKPRAKRGSATDPQSIYARQRRERINERLRALQGLVPNGAKVDIVTML
EEAINYVKFLQLQVKLLSSDEYWMYAPT
Selaginella moelendorfii SmRSLh nucleotide sequence (SEQ ID NO: 57)
CTAAACACCGACGGAAAGCCACGCGCAAAGCGTGGATCTGCCACGGACC
CGCAAAGTATCTACGCTCGGCAAAGAAGAGAAAGGATCAACGAGCGTTT
GAGAGCGCTACAAGGACTCGTACCAAACGGAGCGAAGGTTGACATTGTG
ACGATGCTCGAGGAAGCCATCAACTATGTCAAGTTTTTGCAGCTGCAAGT
AAAGCTGCTCAGCTCGGACGAGTATTGGATGTACGCCCCCACA
Rice (Oryza sativa subsp. Japonica) OsRSLa amino acid sequence (SEQ ID NO: 58;
LOC_Os01g02110.1 11971.m06853)
MMAAQASSKRGMLLPREAVLYDDEPSMPLEILGYHGNGVGGGGCVDADYY
YSWSGSSSSSSSSVLSFDQAAVGGSGGGCARQLAFHPGGDDDDCAMWMDA
AAGAMVENTSVVAGGGNNYCHRLQFHGGAAGFGLASPGSSVVDNGLEIHES
NVSKPPPPAAKKRACPSGEARAAGKKQCRKGSKPNKAASASSPSPSPSPSPSP
NKEQPQSAAAKVRRERISERLKVLQDLVPNGTKVDLVTMLEKAINYVKFLQL
QVKVLATDEFWPAQGGKAPELSQVKDALDAILSSQHPNK*
Rice OsRSLa nucleotide sequence (SEQ ID NO: 59; LOC_Os01g02110.1
11971.m06853)
ATGATGGCAGCTCAGGCAAGCAGCAAGCGCGGCATGCTGCTGCCACGGG
AGGCGGTGCTCTACGACGACGAGCCCTCCATGCCGCTGGAGATCTTGGGC
TACCACGGCAATGGCGTCGGCGGCGGTGGCTGCGTTGACGCCGATTACTA
CTACAGCTGGTCGGGGTCCAGCTCCAGCTCCAGCTCGTCGGTGCTCAGCT
TTGACCAGGCGGCGGTCGGCGGCAGCGGCGGCGGCTGCGCCCGGCAGCT
GGCTTTCCATCCCGGCGGCGACGACGACGACTGCGCCATGTGGATGGACG
CCGCCGCCGGCGCCATGGTCGAGAACACGTCTGTCGTCGCCGGCGGCGGC
AACAACTACTGTCATCGCCTGCAGTTCCACGGCGGCGCCGCCGGTTTCGG
ACTCGCGAGCCCAGGCTCGTCGGTCGTTGACAACGGCCTCGAAATCCACG
AGAGCAACGTCAGCAAGCCGCCACCGCCGGCAGCCAAGAAGCGCGCATG
CCCGAGCGGCGAGGCGAGAGCAGCGGGGAAGAAGCAGTGCAGGAAAGG
GAGCAAGCCAAACAAGGCTGCTTCTGCTTCTTCTCCTTCTCCTTCTCCTTC
TCCTTCTCCTTCTCCTAACAAGGAACAACCTCAAAGCGCCGCTGCAAAGG
TAAGAAGAGAGCGGATCAGTGAGAGGCTCAAAGTTCTTCAGGATCTCGTG
CCTAATGGCACAAAGGTAGACTTGGTCACCATGCTAGAAAAGGCGATCAA
CTACGTCAAATTCCTCCAGCTGCAAGTGAAGGTTTTGGCTACTGATGAGTT
CTGGCCGGCACAAGGAGGGAAAGCACCAGAGCTCTCTCAAGTCAAGGAC
GCCTTGGACGCCATCCTATCTTCTCAGCATCCAAACAAATGA
Rice OsRSLb amino acid sequence (SEQ ID NO: 60; LOC_Os02g48060.1
11972.m09840)
MRMALVRERAMVYGGGCDAEAFGGGFESSQMGYGHDALLDIDAAALFGG
YEAAASAGCALVQDGAAGWAGAGASSSVLAFDRAAQAEEAECDAWIEAM
DQSYGAGGEAAPYRSTTAVAFDAATGCFSLTERATGGGGGAGGRQFGLLFP
STSGGGVSPERAAPAPAPRGSQKRAHAESSQAMSPSKKQCGAGRKAGKAKS
APTTPTKDPQSLAAKNRRERISERLRILQELVPNGTKVDLVTMLEKAISYVKF
LQLQVKVLATDEFWPAQGGKAPEISQVKEALDAILSSSSPLMGQLMN*
Rice OsRSLb nucleotide sequence (SEQ ID NO: 61; LOC_Os02g48060.1
11972.m09840)
ATGCGCATGGCGCTGGTGCGGGAGCGCGCGATGGTGTACGGTGGAGGGT
GCGACGCCGAGGCGTTCGGCGGCGGGTTCGAGTCGTCCCAGATGGGGTAC
GGCCACGACGCGCTGCTCGACATCGACGCGGCGGCGCTGTTCGGGGGGTA
CGAGGCGGCCGCCAGCGCCGGGTGCGCCCTCGTGCAGGACGGCGCCGCG
GGGTGGGCGGGCGCGGGCGCGTCGTCCTCGGTGCTGGCGTTCGACCGCGC
CGCTCAGGCGGAGGAGGCCGAGTGCGACGCGTGGATCGAAGCCATGGAC
CAGAGCTACGGCGCCGGCGGCGAGGCGGCGCCGTACCGGTCGACGACGG
CCGTCGCCTTCGACGCGGCCACCGGCTGCTTCAGCCTGACGGAGAGAGCC
ACCGGCGGCGGCGGCGGCGCGGGTGGGCGGCAGTTCGGGCTGCTGTTCCC
GAGCACGTCGGGCGGCGGCGTCTCCCCCGAACGCGCCGCGCCGGCGCCG
GCGCCCCGCGGCTCGCAGAAGCGGGCCCACGCGGAGTCGTCGCAGGCCA
TGAGCCCTAGCAAGAAGCAGTGCGGCGCCGGCAGGAAGGCGGGCAAGGC
CAAGTCGGCGCCGACCACCCCAACCAAGGACCCGCAAAGCCTCGCGGCC
AAGAATCGGCGCGAGAGGATCAGCGAGCGGCTGCGGATCCTGCAGGAGC
TCGTGCCCAACGGCACCAAGGTCGACCTCGTCACCATGCTCGAGAAGGCC
ATCAGCTACGTCAAGTTCCTCCAGCTTCAAGTCAAGGTTCTTGCGACGGA
CGAGTTCTGGCCGGCGCAGGGAGGGAAGGCGCCGGAGATATCCCAGGTG
AAGGAGGCGCTCGACGCCATCTTGTCGTCGTCGTCGCCGCTGATGGGACA
ACTCATGAACTGA
Rice OsRSLc amino acid sequence (SEQ ID NO: 62; LOC_Os06g30090.1
11976.m07553)
MAMVAGDEAMSVPWHDVGVVVDPEAAGTAPFDAGAGYVPSYGQCQYYY
YYDDHHHHPCSTELIHAGDAGSAVAVAYDGVDGWVHAAAAATSPSSSSALT
FDGHGAEEHSAVSWMDMDMDAHGAAPPLIGYGPTAATSSPSSCFSSGGSGD
SGMVMVTTTTPRSAAASGSQRRARPPPSPLQGSELHEYSKKQRANNKETQSS
AAKSRRERISERLRALQELVPSGGKVDMVTMLDRAISYVKFMQMQLRVLET
DAFWPASDGATPDISRVKDALDAIILSSSSPSQKASPPRSG*
Rice OsRSLc nucleotide sequence (SEQ ID NO: 63; LOC_Os06g30090.1
11976.m07553)
ATGGCTATGGTGGCCGGCGACGAGGCGATGTCAGTGCCATGGCACGACGT
CGGCGTCGTCGTCGACCCCGAGGCGGCCGGGACGGCGCCGTTCGACGCCG
GCGCCGGCTATGTCCCATCGTACGGTCAGTGCCAATACTACTACTACTAC
GACGACCACCACCACCACCCGTGCAGCACGGAGCTGATCCACGCGGGCG
ACGCTGGCAGTGCGGTTGCGGTTGCGTACGACGGCGTCGACGGCTGGGTT
CACGCCGCCGCCGCAGCCACCTCCCCGTCCTCGTCATCTGCGCTCACCTTC
GATGGTCACGGCGCCGAGGAGCACAGCGCAGTGTCGTGGATGGACATGG
ACATGGACGCGCACGGCGCCGCGCCTCCCCTAATCGGCTACGGCCCGACG
GCGGCGACCTCCTCCCCCTCCTCCTGCTTCAGCTCCGGCGGCTCCGGCGAC
AGCGGCATGGTGATGGTGACCACCACCACCCCGAGGAGCGCCGCCGCCTC
TGGTTCGCAGAGGCGGGCACGCCCGCCGCCGTCGCCGTTGCAGGGATCAG
AGCTGCACGAGTACTCCAAGAAGCAGCGCGCCAACAACAAGGAGACACA
GAGCTCAGCTGCCAAGAGCCGGCGGGAGAGGATCAGCGAGCGGCTGAGG
GCGCTGCAGGAGCTGGTGCCGAGCGGCGGGAAGGTGGACATGGTGACCA
TGCTGGACAGGGCCATCAGCTACGTCAAGTTCATGCAGATGCAGCTCAGG
GTGCTGGAGACCGACGCGTTCTGGCCGGCGTCCGACGGCGCCACGCCGGA
CATCTCCCGGGTCAAGGACGCGCTCGACGCCATCATCCTCTCCTCGTCCTC
GCCCTCGCAAAAGGCTTCTCCTCCTCGGTCGGGCTAG
Rice OsRSLd amino acid sequence (SEQ ID NO: 64; LOC_Os03g10770.1
11973.m06529)
MEDSEAMAQLLGVQYFGNDQEQQQPAAAAPPAMYWPAHDAADQYYGSAP
YCYMQQQQHYGCYDGGAMVAGGDFFVPEEQLVADPSFMVDLNLEFEDQH
GGDAGGAGSSAAAAAAATKMTPACKRKVEDHKDESCTDNVARKKARSTA
ATVVQKKGNKNAQSKKAQKGACSRSSNQKESNGGGDGGNVQSSSTNYLSD
DDSLSLEMTSCSNVSSASKKSSLSSPATGHGGAKARAGRGAATDPQSLYARK
RRERINERLKILQNLIPNGTKVDISTMLEEAVHYVKFLQLQIKLLSSDDMWMF
APIAYNGVNVGLDLKISPPQQQ*
Rice OsRSLd nucleotide sequence (SEQ ID NO: 65; LOC_Os03g10770.1
11973.m06529)
ATGGAGGACTCGGAGGCGATGGCGCAGCTGCTCGGCGTGCAGTACTTCGG
CAATGACCAGGAGCAGCAGCAGCCGGCGGCGGCGGCGCCGCCGGCGATG
TACTGGCCGGCGCACGACGCGGCCGACCAGTACTACGGCTCGGCGCCATA
CTGCTACATGCAGCAGCAGCAGCATTACGGGTGCTACGACGGCGGCGCG
ATGGTGGCCGGCGGCGACTTCTTCGTGCCGGAGGAGCAGCTGGTGGCCGA
CCCGAGCTTCATGGTGGACCTGAACCTCGAGTTCGAGGACCAGCACGGCG
GCGATGCTGGCGGCGCTGGGAGCAGCGCCGCCGCCGCCGCCGCCGCCAC
CAAGATGACACCGGCGTGCAAGAGGAAGGTTGAGGATCACAAGGATGAG
AGCTGCACGGACAACGTCGCGAGGAAGAAGGCGCGCTCCACGGCAGCAA
CAGTGGTGCAGAAGAAGGGTAATAAGAACGCGCAGTCAAAGAAGGCGCA
GAAGGGCGCGTGCAGCCGGAGCAGCAACCAGAAGGAGAGCAATGGCGG
CGGCGACGGCGGCAATGTGCAGAGCTCGAGCACCAACTACCTCTCTGATG
ACGACTCGCTGTCGCTGGAGATGACTTCGTGCAGCAACGTGAGCTCGGCG
TCCAAGAAGTCGTCGTTGTCATCGCCGGCGACCGGGCACGGCGGCGCGAA
GGCGAGGGCCGGGCGCGGGGCGGCGACCGATCCGCAAAGCCTCTATGCC
AGGAAGAGGAGAGAAAGGATCAATGAACGGCTAAAGATACTGCAGAATC
TTATCCCAAATGGAACCAAGGTGGACATCAGCACGATGCTTGAAGAAGCA
GTTCACTACGTCAAGTTCTTGCAGCTCCAAATCAAGCTTCTGAGCTCGGAT
GATATGTGGATGTTCGCGCCGATCGCGTACAACGGGGTCAACGTCGGGCT
CGACCTCAAGATCTCTCCACCGCAGCAGCAATGA
Rice OsRSLe amino acid sequence (SEQ ID NO: 66; LOC_Os03g42100.1
11973.m09268)
MESGGVIAEAGWSSLDMSSQAEESEMMAQLLGTCFPSNGEDDHHQELPWSV
DTPSAYYLHCNGGSSSAYSSTTSSNSASGSFTLIAPRSEYEGYYVSDSNEAAL
GISIQEQGAAQFMDAILNRNGDPGFDDLADSSVNLLDSIGASNKRKIQEQGRL
DDQTKSRKSAKKAGSKRGKKAAQCEGEDGSIAVTNRQSLSCCTSENDSIGSQ
ESPVAAKSNGKAQSGHRSATDPQSLYARKRRERINERLKILQNLVPNGTKVDI
STMLEEAMHYVKFLQLQIKLLSSDEMWMYAPIAYNGMNIGIDLNLSQH*
Rice OsRSLe nucleotide sequence (SEQ ID NO: 67; LOC_Os03g42100.1
11973.m09268)
ATGGAGTCCGGAGGGGTGATCGCGGAGGCGGGGTGGAGCTCGCTCGACA
TGTCGTCGCAGGCCGAGGAGTCGGAGATGATGGCGCAGCTGCTTGGAACC
TGCTTCCCCTCCAATGGCGAGGATGATCATCACCAAGAGCTTCCTTGGTC
GGTTGACACCCCCAGTGCCTACTACCTCCATTGCAATGGAGGTAGCTCAA
GTGCATACAGCTCTACCACTAGCAGCAACAGTGCTAGTGGTAGCTTCACT
CTCATTGCACCAAGATCTGAGTATGAGGGGTACTATGTGAGTGACTCTAA
TGAGGCGGCCCTCGGGATCAGCATCCAGGAGCAAGGTGCAGCTCAGTTCA
TGGATGCCATTCTCAACCGGAACGGCGATCCGGGCTTCGATGATCTCGCT
GACTCGAGCGTTAATCTGCTGGATTCCATCGGCGCTTCTAACAAGAGAAA
GATTCAGGAGCAAGGCAGGCTAGATGACCAAACGAAAAGTAGGAAATCT
GCGAAGAAGGCTGGCTCGAAGCGGGGAAAGAAGGCGGCGCAATGTGAAG
GTGAAGATGGCAGCATTGCTGTCACCAACAGGCAAAGCTTGAGCTGCTGC
ACCTCTGAAAATGATTCGATTGGTTCTCAAGAATCTCCTGTTGCTGCTAAG
TCGAATGGCAAGGCTCAATCTGGCCATCGGTCAGCAACCGATCCCCAGAG
CCTCTATGCAAGGAAAAGAAGAGAGAGGATCAATGAGAGGCTCAAGATT
CTGCAGAACCTTGTACCAAATGGAACCAAAGTAGATATCAGCACTATGCT
TGAAGAGGCAATGCATTACGTGAAGTTCTTGCAGCTTCAAATCAAGCTCC
TCAGCTCTGATGAAATGTGGATGTACGCACCGATTGCTTACAACGGGATG
AACATCGGGATCGATTTGAACCTCTCTCAGCATTGA
Rice OsRSLf amino acid sequence (SEQ ID NO: 68; LOC_Os11g41640.1
11981.m08005)
MDARCANIWSSADARSEESEMIDQLKSMFWSSTDAEINFYSPDSSVNSCVTTS
TMPSSLFLPLMDDEGFGTVQLMHQVITGNKRMFPMDEHFEQQQKKPKKKTR
TSRSVSSSSTITDYETSSELVNPSCSSGSSVGEDSIAATDGSVVLKQSDNSRGH
KQCSKDTQSLYAKRRRERINERLRILQQLVPNGTKVDISTMLEEAVQYVKFL
QLQIKLLSSDDTWMFAPLAYNGMNMDLGHTLAENQE*
Rice OsRSLf nucleotide sequence (SEQ ID NO: 69; LOC_Os11g41640.1
11981.m08005)
ATGGATGCAAGGTGTGCAAACATCTGGAGCTCTGCTGATGCAAGGAGTGA
GGAATCTGAGATGATTGATCAACTAAAGTCCATGTTCTGGAGCAGCACTG
ATGCTGAAATCAACTTTTATTCTCCTGACAGTAGTGTAAATTCTTGTGTCA
CAACTAGCACAATGCCTAGCAGCTTGTTTCTTCCTCTGATGGATGATGAGG
GATTTGGCACAGTGCAATTGATGCATCAGGTCATCACTGGGAACAAGAGG
ATGTTCCCCATGGATGAGCACTTTGAGCAGCAGCAGAAGAAGCCGAAGA
AGAAAACCCGAACTTCTCGCTCGGTATCAAGTAGTTCAACCATTACTGAC
TATGAGACTAGCTCTGAACTTGTCAATCCTAGCTGTTCCTCCGGGAGCAGC
GTCGGAGAGGATTCAATTGCTGCAACTGATGGATCTGTAGTGCTGAAACA
AAGTGACAATTCAAGAGGCCATAAGCAGTGCTCCAAGGATACACAAAGC
CTCTATGCTAAGAGGAGAAGGGAAAGGATTAATGAGAGACTGAGAATAC
TTCAGCAGCTTGTTCCCAATGGCACTAAAGTTGACATCAGCACAATGCTG
GAGGAAGCAGTTCAGTATGTCAAGTTTTTGCAGTTGCAAATAAAGCTATT
GAGCTCTGACGACACATGGATGTTTGCGCCCCTAGCCTATAATGGCATGA
ACATGGATCTCGGTCATACTCTTGCTGAAAACCAAGAATGA
Rice OsRSLg amino acid sequence (SEQ ID NO: 70; LOC_Os12g32400.1
11982.m07043)
MECSSFEAICNESEMIAHLQSLFWSSSDADPCFGSSSFSLISSEGYDTMTTEFV
NSSTNVCFDYQDDSFVSAEETTIGNKRKVQMDTENELMTNRSKEVRTKMSV
SKACKHSVSAESSQSYYAKNRRQRINERLRILQELIPNGTKVDISTMLEEAIQY
VKFLHLQIKLLSSDEMWMYAPLAFDSGNNRLYQNSLSQE*
Rice OsRSLg nucleotide sequence (SEQ ID NO: 71; LOC_Os12g32400.1
11982.m07043)
ATGGAATGCAGCTCCTTTGAAGCAATCTGCAATGAGTCGGAGATGATTGC
GCATTTGCAGTCATTGTTCTGGAGCAGCAGCGATGCTGATCCTTGTTTTGG
TAGCTCATCATTTTCTCTCATCAGTAGTGAGGGCTACGACACAATGACCAC
AGAGTTTGTGAATAGCAGCACAAATGTATGTTTTGATTACCAAGATGATA
GCTTCGTTTCAGCAGAGGAGACTACCATTGGTAACAAGAGAAAAGTTCAG
ATGGATACTGAGAATGAGCTGATGACGAACCGCAGCAAGGAAGTTCGCA
CCAAGATGTCGGTGTCAAAAGCATGCAAACATTCTGTTTCTGCAGAGAGC
TCACAGTCTTATTATGCAAAGAACAGGAGACAGAGGATCAATGAGAGATT
GAGAATACTGCAAGAACTGATCCCTAATGGAACAAAAGTTGACATCAGC
ACAATGTTGGAGGAAGCAATTCAGTATGTCAAGTTTCTACACCTGCAAAT
CAAGCTCTTGAGCTCTGATGAAATGTGGATGTATGCGCCCCTTGCTTTTGA
CAGTGGTAACAACAGGCTCTATCAGAACTCTCTGTCACAAGAGTAG
Rice OsRSLh amino acid sequence (SEQ ID NO: 72; LOC_Os12g39850.1
11982.m07769)
MEGGGLIADMSWTVFDLPSHSDESEMMAQLFSAFPIHGEEEGHEQLPWFDQS
SNPCYYSCNASSTAYSNSNASSIPAPSEYEGYCFSDSNEALGVSSSIAPHDLSM
VQVQGATEFLNVIPNHSLDSFGNGELGHEDLDSVSGTNKRKQSAEGEFDGQT
RGSKCARKAEPKRAKKAKQTVEKDASVAIPNGSCSISDNDSSSSQEVADAGA
TSKGKSRAGRGAATDPQSLYARKRRERINERLKTLQNLVPNGTKVDISTMLE
EAVHYVKFLQLQIKLLSSDEMWMYAPIAYNGMNIGLDLNIDT*
Rice OsRSLh nucleotide sequence (SEQ ID NO: 73; LOC_Os12g39850.1
11982.m07769)
ATGGAGGGTGGAGGACTGATCGCCGATATGAGCTGGACCGTCTTCGACTT
GCCATCGCACAGCGATGAGTCGGAGATGATGGCGCAGCTCTTCAGTGCAT
TCCCCATCCATGGTGAGGAGGAAGGCCATGAGCAGCTCCCATGGTTTGAT
CAATCTTCCAATCCATGCTACTATAGCTGCAATGCTAGCAGCACTGCATA
CAGCAACAGCAATGCTAGTAGCATTCCTGCTCCATCTGAGTATGAAGGAT
ACTGCTTCAGTGACTCAAATGAGGCCCTGGGTGTCAGCTCCAGCATTGCA
CCACATGACCTGAGCATGGTCCAGGTGCAAGGTGCAACTGAGTTTCTGAA
TGTGATCCCAAACCATTCCCTTGATTCATTCGGTAATGGCGAGCTGGGCCA
CGAGGATCTTGATTCGGTTAGTGGGACTAACAAGAGAAAACAGTCGGCA
GAAGGAGAATTTGATGGCCAAACAAGAGGTTCAAAATGCGCGAGAAAGG
CTGAACCGAAGCGAGCGAAGAAGGCCAAGCAAACTGTGGAGAAGGATGC
AAGTGTTGCCATCCCAAATGGGAGCTGTTCCATTTCTGACAATGATTCCAG
TTCATCCCAGGAGGTTGCAGATGCTGGTGCTACTTCGAAAGGCAAATCCC
GGGCTGGCCGCGGAGCAGCCACTGATCCCCAGAGCCTCTATGCAAGGAA
AAGGAGAGAGAGGATCAATGAGAGGCTCAAGACACTTCAGAACCTTGTG
CCCAATGGCACCAAAGTTGATATCAGCACCATGCTTGAGGAGGCAGTCCA
CTATGTGAAGTTCCTGCAGCTTCAGATCAAGCTCCTCAGCTCCGATGAAAT
GTGGATGTATGCGCCAATTGCGTACAACGGGATGAACATTGGGCTCGATC
TGAACATTGATACATGA
Rice OsRSLi amino acid sequence (SEQ ID NO: 74; LOC_Os07g39940.1
11977.m08236)
MAQFLGAHGDHCFTYEQMDESMEAMAAMFLPGLDTDSNSSSGCLNYDVPP
QCWPQHGHSSSVTSFPDPAHSYGSFEFPVMDPFPIADLDAHCAIPYLTEDLISP
PHGNHPSARVEEATKVVTPVATKRKSSAAMTASKKSKKAGKKDPIGSDEGG
NTYIDTQSSSSCTSEEGNLEGNAKPSSKKMGTRANRGAATDPQSLYARKRRE
RINERLRILQNLVPNGTKVDISTMLEEAVQYVKFLQLQIKLLSSDDTWMYAPI
AYNGVNISNIDLNISSLQK*
Rice OsRSLi nucleotide sequence (SEQ ID NO: 75; LOC_Os07g39940.1
11977 .m08236)
ATGGCGCAGTTTCTTGGAGCTCATGGTGATCACTGCTTCACCTACGAGCA
AATGGATGAGTCCATGGAGGCAATGGCAGCGATGTTCTTGCCTGGCCTTG
ACACCGACTCCAATTCTTCTTCTGGTTGTCTCAACTACGATGTGCCTCCAC
AATGCTGGCCTCAGCATGGCCATAGCTCTAGCGTCACCAGCTTCCCTGAT
CCAGCTCATAGCTATGGAAGCTTTGAGTTCCCGGTCATGGATCCGTTCCCG
ATCGCCGATCTCGACGCGCATTGCGCCATCCCCTACCTTACTGAGGATCTG
ATCAGCCCTCCACATGGCAACCATCCATCAGCAAGAGTGGAAGAAGCTAC
AAAGGTTGTTACACCAGTGGCTACCAAGAGGAAGTCTAGTGCTGCCATGA
CGGCATCAAAGAAGAGCAAGAAGGCTGGCAAAAAAGATCCTATTGGCAG
CGACGAAGGCGGCAACACCTACATTGATACGCAAAGTTCTAGCAGTTGCA
CCTCAGAGGAAGGAAACCTGGAGGGCAACGCGAAGCCGAGCTCGAAGAA
GATGGGTACTAGGGCCAACCGTGGGGCGGCAACCGATCCCCAGAGTCTCT
ATGCAAGGAAGAGGAGAGAGAGGATCAATGAAAGATTGAGGATCCTGCA
GAACTTGGTTCCCAATGGAACAAAGGTTGACATCAGTACAATGCTGGAGG
AAGCAGTGCAGTATGTCAAATTTTTGCAACTTCAGATTAAGTTGCTAAGCT
CTGATGACACGTGGATGTATGCACCAATCGCTTACAATGGAGTCAACATC
AGCAATATTGATCTGAACATCTCTTCTCTGCAAAAATAA
Populus trichocarpa PtRSLa amino acid sequence (SEQ ID NO: 76)
MALAKDRMGSVQTCPYNGNVMGDFSSMGSYGFDEYQKVAFYEEGNSTFEK
TSGLMIKNLAMTSSPSSLGSPSSAISGELVFQATDHQAEEAHSLISFKGIGFDNI
MHNNGSLLSFEQSSRVSQTSSQKDDYSAWEGNLSYNYQWNEMNPKCNTSPR
LMEDFNCFQRAGNFISMTGKENHGDWLYAESTIVADSIQDSATPDASSFHKR
PNMGESMQALKKQCNNATKKPKPKSAAGPAKDLQSIAAKNRRERISERLKV
LQDLVPNGSKVDLVTMLEKAISYVKFLQLQVKVLATDELWPVQGGKAPDIS
QVKEAIDALLSSQTKDGNSSSSPK*
Populus trichocarpa PtRSLa nucleotide sequence (SEQ ID NO: 77)
ATGGCACTTGCCAAGGACCGTATGGGATCGGTTCAAACTTGCCCCTATAA
TGGAAATGTGATGGGGGATTTTTCCTCCATGGGGTCTTACGGATTTGATGA
ATATCAGAAGGTAGCATTTTATGAAGAGGGAAATAGCACCTTTGAGAAAA
CCAGTGGGCTTATGATCAAGAATTTAGCTATGACCTCTTCTCCTTCTTCTC
TTGGCAGTCCGAGCAGCGCGATTTCTGGTGAATTAGTGTTTCAGGCTACTG
ACCATCAAGCTGAGGAAGCTCATTCTTTGATCAGCTTCAAAGGTATCGGA
TTCGATAACATCATGCATAATAATGGATCTTTGCTTAGCTTTGAGCAAAGT
AGTAGGGTTTCTCAAACTAGTAGCCAGAAAGATGACTACTCAGCCTGGGA
GGGTAATTTGAGTTACAACTACCAGTGGAACGAAATGAATCCAAAATGTA
ACACAAGTCCTCGGTTGATGGAAGATTTTAATTGCTTTCAAAGAGCTGGC
AACTTCATTTCCATGACTGGAAAGGAAAATCATGGTGATTGGTTATACGC
TGAATCCACAATTGTTGCTGATAGCATTCAGGATTCTGCAACACCAGATG
CCAGCAGCTTCCATAAGCGTCCTAATATGGGAGAGAGTATGCAGGCTCTA
AAGAAGCAATGCAACAATGCAACAAAAAAGCCAAAACCGAAGTCCGCAG
CAGGTCCAGCTAAGGATCTACAGAGTATTGCTGCCAAGAATCGACGAGAG
AGGATTAGCGAGAGGCTTAAGGTATTGCAGGATTTAGTCCCTAATGGCTC
AAAGGTTGATTTGGTTACTATGCTAGAGAAAGCCATTAGTTATGTTAAGTT
TCTTCAATTGCAAGTAAAGGTGTTAGCCACTGATGAATTATGGCCAGTTC
AAGGTGGTAAAGCTCCTGATATTTCTCAAGTAAAGGAAGCCATCGATGCC
CTACTCTCATCTCAGACTAAAGACGGAAACTCAAGCTCAAGCCCAAAGTA
A
Populus trichocarpa PtRSLb amino acid sequence (SEQ ID NO: 78)
MALAKDRMDSVQTCALYGNVMGDLSSLGPNYRFDEEGDRNFEKNSALMIK
NLAMSPSPPSLGSPSSANSGELVFQATDNQVEEAHSLINFKGTGFDSIMHANG
SLISFEQSNRVSQTSSHKDDYSAWEGNLSCNYQWNQINPKCNANPRLMEDLN
CYQSASNFNSITNSAEKENHGDWLYTHESTIVTDSIPDSATPDASSFHKRPNM
GESMQALKKQRDSATKKPKPKSAGPAKDPQSIAAKNRRERISERLKMLQDLV
PNGSKVDLVTMLEKAISYVKFLQLQVKVLATDEFWPVQGGKAPDISQVKGAI
DATLSSQTKDRNSNSSSK*
Populus trichocarpa PtRSLb nucleotide sequence (SEQ ID NO: 79)
ATGGCACTTGCCAAGGACCGTATGGATTCGGTTCAAACTTGCGCCCTTTAT
GGAAATGTGATGGGGGATCTTTCCTCCTTGGGGCCTAATTATAGATTTGAT
GAAGAGGGAGATAGGAACTTTGAGAAAAATAGTGCGCTTATGATCAAGA
ATTTAGCTATGAGCCCTTCTCCTCCTTCTCTTGGCAGTCCAAGCAGTGCAA
ATTCTGGTGAACTAGTGTTTCAGGCTACTGACAATCAAGTTGAGGAAGCT
CATTCTTTGATCAACTTCAAAGGTACCGGATTTGATAGTATCATGCATGCT
AATGGATCTTTGATTAGCTTTGAGCAAAGTAATAGGGTTTCTCAAACTAGT
AGTCACAAAGATGACTACTCTGCTTGGGAGGGTAATTTGAGTTGCAATTA
CCAGTGGAACCAAATCAATCCAAAATGTAACGCAAATCCTCGGTTGATGG
AAGATCTTAATTGCTATCAAAGTGCAAGCAACTTCAACTCCATAACCAAC
AGTGCTGAAAAGGAAAACCATGGTGATTGGTTATACACTCATGAATCCAC
AATTGTTACTGATAGCATTCCCGATTCTGCAACACCAGATGCCAGCAGCTT
CCATAAGCGTCCCAATATGGGAGAGAGTATGCAGGCTCTAAAGAAGCAA
CGCGACAGCGCCACAAAAAAGCCGAAACCCAAGTCTGCTGGTCCAGCTA
AGGATCCACAAAGTATTGCTGCCAAGAATCGACGAGAGCGGATTAGCGA
GCGCCTTAAGATGTTGCAGGATTTAGTCCCTAACGGCTCCAAGGTTGATTT
GGTTACTATGCTAGAGAAAGCCATTAGTTATGTTAAGTTTCTTCAATTGCA
AGTAAAGGTGTTGGCCACTGATGAATTCTGGCCAGTTCAAGGTGGTAAAG
CTCCTGATATTTCTCAAGTAAAGGGAGCCATTGATGCCACACTCTCATCTC
AGACTAAAGACAGAAATTCAAACTCAAGCTCAAAGTGA
Populus trichocarpa PtRSLc amino acid sequence (SEQ ID NO: 80)
MAEGEWSSLGGMYTSEEADFMAQLLGNCPNQVDSSSNFGVPSSFWPNHEPT
TDMEGANECLFYSLDFANINLHHFSQGSSSYSGGSGILFPNTSQDSYYMSDSH
PILANNNSSMSMDFCMGDSYLVEGDDCSNQEMSNSNEEPGGNQTVAALPEN
DFRAKREPEMPASELPLEDKSSNPPQISKKRSRNSGDAQKNKRNASSKKSQK
VASTSNNDEGSNAGLNGPASSGCCSEDESNASHELNRGASSSLSSKGTATLNS
SGKTRASRGAATDPQSLYARKRRERINERLRILQTLVPNGTKVDISTMLEEAV
QYVKFLQLQIKLLSSEDLWMYAPIAYNGMDIGLDHLKVTAP*
Populus trichocarpa PtRSLc nucleotide sequence (SEQ ID NO: 81)
ATGGCAGAGGGAGAGTGGAGTTCTCTTGGTGGAATGTACACTAGTGAGGA
GGCTGATTTCATGGCACAGTTGCTTGGTAACTGTCCTAATCAGGTTGATTC
AAGTTCAAACTTTGGAGTTCCATCTAGTTTCTGGCCTAACCACGAACCAAC
AACGGACATGGAAGGGGCTAATGAATGTTTATTTTATTCTTTGGATTTTGC
TAATATTAATTTGCACCATTTTTCACAAGGGAGTAGTAGTTATAGTGGTGG
CAGTGGCATTCTTTTTCCCAACACAAGCCAAGATAGCTACTACATGAGTG
ATTCTCATCCAATTTTGGCTAACAATAATAGCTCAATGTCAATGGATTTTT
GCATGGGAGACTCATATCTCGTTGAAGGCGATGACTGCTCAAACCAAGAA
ATGAGCAATAGCAATGAGGAGCCTGGTGGAAACCAGACTGTAGCTGCTCT
TCCTGAAAACGATTTTCGGGCCAAGAGAGAACCAGAGATGCCAGCTTCTG
AACTACCCCTGGAAGACAAAAGCAGCAACCCACCTCAGATTTCTAAGAA
AAGATCACGAAATTCAGGAGATGCTCAAAAGAACAAGAGGAATGCAAGT
TCAAAGAAGAGCCAGAAGGTTGCCTCGACTAGCAACAATGATGAAGGAA
GTAATGCTGGCCTTAATGGGCCTGCCTCAAGCGGTTGCTGCTCAGAGGAT
GAATCCAATGCCTCTCATGAGCTCAATAGAGGAGCGAGTTCAAGTTTGAG
CTCGAAAGGGACTGCAACTCTCAACTCAAGTGGCAAAACAAGAGCCAGC
AGGGGGGCAGCCACTGATCCCCAGAGTCTCTATGCAAGGAAAAGAAGAG
AAAGAATAAATGAGAGGCTGAGAATTCTACAAACCCTTGTCCCCAACGGA
ACAAAGGTTGACATTAGCACAATGCTTGAAGAAGCTGTCCAGTATGTGAA
GTTTTTGCAACTCCAAATTAAGCTGCTAAGCTCTGAGGACTTGTGGATGTA
TGCGCCTATCGCTTACAACGGGATGGACATCGGTCTTGATCATCTGAAGG
TTACCGCACCATGA
Populus trichocarpa PtRSLd amino acid sequence (SEQ ID NO: 82)
MEPIGATAEGEWSSLSGMYTSEEADFMEQLLVNCPPNQVDSSSSFGVPSSFW
PNHESTMNMEGANECLLYSLDIADTNLYHFSQVSSGYSGELSNGNVEESGGN
QTVAALPEPESNLQPKRESKMPASELPLEDKSRKPPENSKKRSRRTGDAQKN
KRNVRSKKSQKVASTGNNDEESNGGLNGPVSSGCCSEDESNASQELNGGASS
SLSSKGTTTLNSSGKTRASKGAATDPQSLYARKRRERINERLRILQNLVPNGT
KVDISTMLEEAVQYVKFLQLQIKLLSSEDLWMYAPIAYNGMDIGLDHLKLTT
PRRL*
Populus trichocarpa PtRSLd nucleotide sequence (SEQ ID NO: 83)
ATGGAGCCTATTGGAGCCACTGCGGAGGGAGAGTGGAGTTCTCTTAGTGG
AATGTACACAAGTGAGGAGGCTGATTTCATGGAACAGTTGCTTGTCAACT
GTCCTCCTAATCAGGTTGATTCAAGTTCAAGCTTTGGAGTTCCATCTAGTT
TTTGGCCTAACCATGAATCAACAATGAACATGGAAGGGGCCAATGAATGT
TTATTGTATTCTTTGGATATTGCTGATACTAATCTGTACCATTTTTCACAAG
TGAGCAGTGGTTATAGTGGTGAATTGAGCAATGGAAATGTGGAAGAGTCT
GGTGGAAACCAGACTGTAGCTGCTCTTCCTGAACCTGAAAGCAATTTGCA
ACCCAAGAGAGAATCAAAGATGCCAGCATCTGAACTACCCCTGGAAGAT
AAAAGCAGAAAGCCACCTGAGAATTCCAAGAAAAGATCACGACGTACGG
GAGATGCCCAAAAGAACAAGAGGAATGTAAGGTCAAAGAAGAGCCAGA
AGGTTGCCTCGACTGGCAACAATGATGAAGAAAGCAATGGTGGCCTTAAT
GGTCCTGTCTCAAGCGGTTGCTGCTCAGAGGATGAATCCAATGCCTCCCA
GGAGCTCAATGGAGGAGCGAGTTCAAGTTTGAGCTCAAAAGGGACAACA
ACTCTCAACTCAAGTGGCAAAACAAGAGCCAGTAAGGGGGCAGCCACTG
ATCCCCAGAGCCTCTATGCAAGGAAAAGAAGAGAAAGAATAAATGAGAG
GCTGAGAATTCTACAAAACCTTGTCCCCAATGGAACAAAGGTTGACATTA
GCACAATGCTTGAAGAGGCTGTCCAGTATGTGAAGTTTTTGCAACTCCAA
ATTAAGCTGCTAAGCTCTGAAGACCTGTGGATGTATGCTCCTATCGCGTAC
AATGGTATGGACATCGGTCTTGATCATCTGAAGCTTACCACACCAAGACG
ATTGTAG
Populus trichocarpa PtRSLe amino acid sequence (SEQ ID NO: 84)
MNTQAMEAFRDGELWNFSRMFSMEEPDCTPELLGQCSFLQDTDEGLHFTIPS
AFFPAPESDASMAEDESLFYSWHTPNPNLHFDSQESSNNSNSSSSVFLPYSSHE
SYFFNDSNPIQATNNNSMSMDIMDEENIGLFMPLFPEIAMAETACMNGDMSG
DKTGDLDDNLKPAANDVLAKGLQLKRKLDVPEPIANTLDDMKKKARVTRN
VQKTRKVGQSKKNQKNAPDISHDEEESNAGPDGQSSSSCSSEEDNASQDSDS
KVSGVLNSNGKTRATRGAATDPQSLYARKRRERINERLKILQNLVPNGTKVD
ISTMLEEAVHYVNFLQLQIKLLSSDDLWMYAPLAYNGIDIGLNQKLSMFL*
Populus trichocarpa PtRSLe nucleotide sequence (SEQ ID NO: 85)
ATGAATACGCAGGCTATGGAAGCCTTTCGTGATGGAGAATTATGGAACTT
CAGCAGAATGTTCTCCATGGAAGAGCCTGATTGCACCCCAGAATTACTTG
GTCAGTGCTCTTTTCTTCAGGATACTGATGAAGGATTGCATTTTACAATCC
CATCAGCTTTCTTCCCTGCTCCTGAATCCGACGCGAGCATGGCTGAGGAC
GAGAGTTTGTTTTATTCTTGGCATACTCCCAACCCCAATTTGCATTTTGATT
CTCAAGAAAGTAGTAATAACAGTAATTCTAGCAGTAGTGTATTTCTTCCCT
ATTCCAGCCATGAATCCTACTTCTTCAATGATTCTAATCCCATTCAAGCTA
CGAACAATAACTCTATGTCCATGGATATTATGGATGAGGAAAATATTGGC
TTGTTTATGCCACTTTTTCCTGAAATTGCAATGGCAGAAACTGCCTGTATG
AATGGAGATATGAGCGGTGACAAAACAGGAGATTTAGATGATAATCTGA
AGCCAGCAGCTAATGATGTTCTGGCCAAGGGATTGCAGCTCAAAAGGAA
GCTTGATGTTCCAGAACCAATAGCCAACACATTGGACGACATGAAGAAAA
AAGCCCGGGTTACAAGAAATGTGCAAAAGACTAGGAAGGTTGGACAGTC
AAAAAAAAATCAGAAGAACGCACCAGATATTAGCCATGATGAAGAAGAG
AGTAATGCTGGACCAGACGGACAAAGTTCCAGCAGTTGTAGTTCAGAAGA
GGACAATGCCTCTCAGGATTCTGATTCCAAGGTTTCTGGAGTTCTCAATTC
CAATGGAAAAACAAGAGCTACTAGGGGAGCTGCCACAGACCCCCAGAGC
CTTTATGCAAGGAAAAGAAGGGAGAGGATAAACGAGAGACTGAAAATCT
TGCAGAATCTTGTCCCTAACGGAACCAAGGTTGATATCAGCACGATGCTA
GAAGAGGCAGTCCATTACGTAAACTTTTTGCAGCTTCAAATCAAGCTTTTG
AGCTCGGATGATCTATGGATGTATGCACCTCTGGCTTACAATGGAATAGA
TATTGGACTCAACCAGAAGCTCTCTATGTTTCTATGA
Musa acuminata MaRSLa amino acid sequence (SEQ ID NO: 86; GI102139852,
ABF70010.1)
MAQESTWSSFDATMLAEEESRMIAQLLSNYQCFGEQDRDVGCCELPPSSCCS
SHAADSCYCWSANENSNPGLCYWSQSGDESDGAHAIGTVPVFTNHCLVGDQ
VAVNQTLSIHEPTAAHAEMPKRKIESHASEDDFRRQSSKKKLQAPTNALKSV
KKARPGRNQKSIVCGDEEENNARSSGRSCCSYSSEEDSQAFQADLNAKTRSN
RWPATDPQSLYAKQRRERINARLRTLQNLVPNGTKVDISTMLEEAVRYVKFL
QLQIKLLSSDELWMYAPVVHSGMIDGQVNSEIFVSANTRNEWF*
Musa acuminata MaRSLa nucleotide sequence (SEQ ID NO: 87).
atggctcaggagtcaacttggagctcgtttgatgctacaatgcttgctgaggaggagtcccgaatgatcgcacaattgctca
gcaactaccagtgttttggcgagcaagatcgagatgttggatgctgtgaactcccgccatcgtcttgttgttcttctcatgcag
ctgattcatgttactgttggtcagcaaatgagaacagtaacccgggtttgtgctactggtctcagagtggagatgaatccgat
ggagcacatgcaatcggcactgtgccggtcttcacgaaccattgcttggtgggagatcaagtcgctgtgaatcaaactttga
gcattcacgaacctactgctgctcatgcagagatgccaaagcgcaagatagagtctcatgcttctgaagatgatttccgtcgt
caaagttctaagaaaaagcttcaggctccgacgaatgctctgaagagcgtgaagaaggcacgacctgggaggaaccag
aagagcattgtgtgtggtgatgaggaagagaacaatgccaggagcagtggccggagttgctgcagctacagctctgagg
aagactcacaagctttccaggctgatcttaatgcaaaaacacgatcgaatcgatggccagccacagatcctcaaagcctct
atgcaaagcaaagaagggaaagaatcaatgctagattgaggacattgcagaacctggtgcctaatggaactaaagttgac
attagcacaatgctcgaagaagctgttcgttacgtcaagttcttgcagctgcagataaagcttttgagctcggatgagctgtg
gatgtacgctcctgttgtccacagtgggatgattgatggccaagtcaactcagagatatttgtgtctgcaaatactcgtaatga
gtggttctga
Medicago truncatula MtRSLa amino acid sequence (SEQ ID NO: 88; AC140548.11
GI: 156231148)
MEPIGTFPEGEWDFFRKMFASEDHEYYSQQFLDQNSLLLGENDGLNNGTQST
FCTAEIGENERMFYSFDHAHIQNSNYIPQTQENSYNSNSSASDDTNYYFSYPN
HVLENNINNCISNDFRMDENLFASSVPSLNEIVMEENVRMNEDSASDDHIVEK
NGYNTQIMEPFDLHTKHEMQMKLKRKLDVIEVEVPVEEKINNNPKKKPRVS
NDGQGCMKNARSKKNHKVIASHEEEMTEEINRGSNGNSSSSNISEDDNASQE
NSGGTTLNSNGKTRASRGSATDPQSLYARKRRERINERLRVLQNLVPNGTKV
DISTMLEEAVNYVKFLQTQIKLLSSDDMWMYAPLAYNGLDLGLNLNLNSSLP
L*
Medicago truncatula nucleotide sequence (SEQ ID NO: 89, AC140548.11
GI: 156231148)
atggaacctataggtactttccctgaaggagaatgggatttctttcgcaaaatgtttgcaagtgaagatcatgaatattactcac
aacaatttcttgatcaaaattcacttcttctaggggaaaatgatgggttgaacaatggaacacagtccacattttgcactgctga
aattggtgaaaatgagcgtatgttttattcttttgatcatgctcatatccaaaactctaactatattcctcaaactcaagagaatag
ttacaatagcaattctagtgctagtgatgatacaaattactattttagttatcctaatcatgtactagaaaataatattaataattgta
tatccaatgattttcgcatggatgagaatttgtttgcttcttctgttccatcccttaatgagattgtaatggaagagaatgtgagaa
tgaatgaagattctgcaagtgatgatcatattgtggagaaaaatggttacaatactcaaataatggaaccttttgatcttcacac
caagcatgagatgcaaatgaagctcaaaaggaaacttgatgtgatagaagtggaggttcccgttgaagaaaaaattaacaa
caatccgaagaaaaaacctcgtgtttcgaatgatggccaaggatgcatgaaaaatgcaaggtcaaagaagaaccacaaa
gttattgctagccatgaagaggagatgacagaagagattaatagaggatcaaatggaaatagttctagtagtaacatttctga
ggatgataatgcttctcaagaaaatagtggaggaactactctcaactcaaatgggaagacaagagctagtagaggatctgc
aacagatccccaaagtctatatgcaaggaaaagaagagagagaataaatgaacgactaagagtcttacaaaatcttgtacc
aaacggaacaaaggttgatatcagtacaatgcttgaagaggcagtcaattatgtgaaatttttacagactcaaatcaagctttt
gagctctgatgatatgtggatgtatgcaccacttgcttacaatggacttgaccttggactcaatctcaacctcaacagctctcta
ccactatga
Soybean GmRSLa amino acid sequence (SEQ ID NO: 90)
(gi|26056905|gb|CA799819.1|CA799819)
XFLCFSQGSSSSTDNSGNNIFSITSSGAYSCDPEANFDSVSMVLCLGDAKFSPH
SFQCDDNSNQQINENTDEESSLDPWKLAIADNNLQAKREYEMMVSEPVEVD
RSRNLENLAKRLKSSIEVSKTLRSAKSGKNSKSASVSNDEDDRSLSLQAQRNS
CFSQSDSNAYLEPNGGASKDPAPPNLHRKSRATTGAATDPQSLYARKRRERI
NERLRILQNLVPNGTKVDISTMLEEAVQYVKFLQLQIKLLS SDDLWMY
Soybean GmRSLa nucleotide sequence (SEQ ID NO: 91)
(gi|26056905|gb|CA799819.1|CA799819)
ATTTTTTGTGTTTCTCACAAGGGAGTAGCTCCAGTACTGATAATAGTGGTA
ATAATATCTTTTCCATTACAAGTAGTGGAGCCTACTCCTGTGATCCAGAAG
CAAACTTTGATTCTGTGTCCATGGTTTTGTGCCTTGGAGATGCCAAATTTA
GTCCCCATAGTTTTCAATGTGATGACAACTCAAACCAACAGATAAATGAA
AACACTGATGAAGAGTCAAGTCTAGACCCATGGAAGTTGGCTATAGCTGA
CAATAATTTGCAGGCTAAGAGGGAGTATGAAATGATGGTTTCTGAACCTG
TAGAAGTGGATAGAAGCAGAAACCTGGAGAACCTAGCAAAAAGACTAAA
GAGTTCAATAGAGGTTTCAAAAACATTGAGGAGTGCTAAATCAGGGAAA
AATTCAAAATCTGCTTCAGTGAGCAACGATGAAGATGATAGAAGCTTGAG
CCTCCAAGCCCAAAGGAATAGCTGTTTTTCACAGAGTGACTCTAATGCTT
ATCTGGAGCCAAATGGAGGGGCATCAAAAGATCCTGCACCTCCCAATTTG
CATAGAAAATCAAGAGCAACTACCGGTGCTGCCACTGATCCACAGAGCCT
CTATGCAAGAAAGAGAAGAGAAAGAATAAATGAAAGGTTGAGAATACTG
CAAAATCTTGTTCCCAACGGAACTAAGGTGGATATCAGCACCATGCTTGA
GGAAGCTGTCCAATACGTGAAGTTTTTACAGCTCCAAATTAAGCTTCTGA
GCTCTGACGATCTGTGGATGTAT
Soybean GmRSLb amino acid sequence (SEQ ID NO: 92)
(gi|15663066|gb|B1700437.1|B1700437)
XNLENLPKRLKSS1EVPKTSRNAKSRKNSKSASTSNDEDDRSLSLQVQRNNSC
FSQSDSNAYLEPNGGASKDPAPPNLDRKSRATTSAAADPQSLYARKRRERIN
ERLRILQNLVPNGTKVDISTMLEEAVQYVKFLQLQIKLLS SEDLWMYAPIVYN
GINIGLDLGISPTKGRSM*
Soybean GmRSLb nucleotide sequence (SEQ ID NO: 93).
(gi|15663066|gb|B1700437.1|B1700437)
GAAACCTGGAGAACCTACCAAAAAGACTAAAGAGCTCAATAGAGGTCCC
AAAAACATCGAGGAATGCTAAATCAAGGAAAAATTCAAAATCTGCTTCA
ACTAGCAACGATGAAGATGATAGAAGCTTGAGCCTCCAAGTCCAAAGGA
ATAATAGCTGTTTTTCACAGAGTGACTCTAATGCTTATCTTGAGCCAAATG
GAGGGGCATCAAAAGATCCTGCACCTCCTAATTTGGATAGAAAATCAAGA
GCAACTACCAGTGCCGCCGCTGATCCACAGAGCCTCTATGCAAGAAAGAG
AAGAGAAAGAATAAATGAAAGGCTGAGAATACTGCAAAATCTTGTCCCC
AACGGAACTAAGGTGGATATCAGCACCATGCTTGAAGAAGCTGTCCAATA
CGTTAAGTTTTTACAGCTCCAAATTAAGCTTCTGAGCTCTGAAGATTTGTG
GATGTATGCTCCAATTGTTTACAATGGAATAAACATTGGACTAGACCTCG
GTATTTCTCCAACCAAAGGAAGATCAATGTGATAGCATAGCAATTAAAGA
GGATATAATATTTCATTAACTTA
Lettuce saligna LsRSLa amino acid sequence (SEQ ID NO: 94)
(gi|83790803|gb|DW051020.1|DW051020 CLLX3812.b1_H18.ab1)
XRSKEAEILSSNGKRKASRGSATDPQSVYARKRRER1NERLRILQNLVPNGTK
VDISTMLEEAVEYVKFLQLQIKLLSSDDMWMYAPIAYDGMDIGLHSTTIPSSS
TR*
Lettuce saligna LsRSLa nucleotide sequence (SEQ ID NO:95)
(gi|83790803|gb|DW051020.1|DW051020 CLLX3812.bl_H18.abl)
TGAGATCAAAAGAGGCTGAAATTCTGAGCTCAAATGGCAAGAGAAAAGC
AAGTAGGGGGTCAGCAACTGATCCACAAAGTGTCTATGCACGGAAAAGA
AGAGAAAGAATTAACGAACGTTTAAGAATATTACAAAATCTTGTTCCTAA
TGGTACAAAGGTTGATATAAGCACAATGCTTGAAGAGGCTGTTGAGTACG
TGAAGTTTTTGCAGCTTCAAATCAAGCTCTTGAGCTCCGATGATATGTGGA
TGTATGCTCCGATTGCATACGATGGAATGGACATTGGGCTTCATTCAACA
ACCATCCCATCATCGTCAACAAGATAATGCAAAGTTGGGCTATCCATATT
GTCACATTTTTGTTGAATAAAAGGCAATCGATAACAAAATTCAAAGTTTA
TAAAGAGTACACATTTATGC
Triticum aestivum TaRSLa amino acid sequence (SEQ ID NO: 96)
(gi|25232820|gb|CA654295.1|CA654295)
MASKRATTRELRAMYDDEPSSMSLELFGYHGVVVDGDDENDDTATALPQLS
FVDNFKGGCGSAADYYSWAYNASGGTPGASSSSTSSVLSFEHAGGAGHQLA
YNSGTGDDDCALWMDSMADHQHGAARFGFMNPGSADVVPEIQESSIKQPA
KSAQKRSSSGGEAQAAAKKQCGGGRKSKAKVVPTKDPQSAVAKVRRERISE
RLKVLQDLVPNGTKVDMVTMLEKAITYVKFLQLQVKVLATDEFWPVQGGK
APELSQVKTALDAILSSQQQP*
Triticum aestivum TaRSLa nucleotide sequence (SEQ ID NO:97)
(gi|25232820|gb|CA654295.1|CA654295)
ATGGCGAGCAAGCGGGCCACCACGCGGGAGCTCCGGGCGATGTACGACG
ACGAGCCCTCCTCCATGTCCCTCGAGCTCTTCGGCTACCATGGCGTGGTCG
TCGACGGTGACGATGAAAACGACGACACTGCCACCGCCCTGCCCCAGCTC
TCCTTCGTCGACAACTTCAAAGGTGGGTGCGGGTCGGCGGCGGACTACTA
CAGCTGGGCGTACAACGCCTCCGGCGGGACGCCGGGCGCCTCCTCCAGCT
CCACCTCGTCGGTGCTCAGCTTTGAGCATGCCGGCGGTGCCGGTCATCAG
CTGGCTTATAATTCCGGCACAGGCGACGATGACTGCGCGCTCTGGATGGA
CAGCATGGCCGATCATCAGCACGGCGCGGCCAGGTTTGGGTTCATGAACC
CAGGGTCGGCCGATGTCGTCCCAGAAATCCAGGAGAGCAGCATCAAGCA
GCCGGCCAAGTCTGCGCAGAAGCGCTCGAGCTCGGGTGGTGAGGCGCAA
GCAGCGGCGAAGAAGCAGTGTGGAGGAGGCAGGAAGAGCAAGGCCAAA
GTTGTCCCTACCAAGGATCCTCAGAGCGCTGTTGCAAAGGTCCGAAGAGA
GCGCATCAGTGAGAGGCTCAAAGTTCTGCAGGATCTTGTACCCAACGGCA
CGAAGGTGGACATGGTCACCATGCTCGAGAAGGCAATCACCTATGTCAAG
TTCCTGCAGCTGCAAGTCAAGGTGTTGGCGACCGACGAGTTCTGGCCGGT
GCAAGGAGGGAAGGCGCCGGAGCTCTCCCAAGTGAAGACCGCGCTGGAC
GCCATCCTTTCTTCCCAGCAGCAACCCTAG
Safflower Carthamus tinctorius CtRSLa amino acid sequence (SEQ ID NO: 98)
(gi|125399878|gb|EL411863.1|EL411863 CFF59477.b1_118.ab1)
DSQIIHPMPCDELHKSLI*LYHIRRRYPYWVFTDGESTSFARPLLNDSRIRGELL
LTLSTTKHCKVTASSMRRSYSMMHDHEKS*KIQRRKSQKLVSKGNESEADH
DAVFGQIMKMCGSDNDSNWPRESSTSPRPKEAANLNSNGKTKANRGSATDP
QSVYARKRRERINERLRILQSLVPNGTKVDISTMLEDAVQYVKFLQLQIKPLS
SDDLWMYAPIAYNGMETGLDSTIPSPR*RLSKVAASFFLKKGKPGA
Safflower Carthamus tinctorius CtRSLa nucleotide sequence (SEQ ID NO: 99)
(gi|125399878|gb|EL411863.1|EL411863 CFF59477.b1_118.ab1)
GATTCACAGATAATCCACCCTATGCCGTGTGATGAACTCCACAAATCCTT
AATTTAATTGTACCACATCAGGCGACGTTATCCATATTGGGTGTTCACTGA
TGGTGAAAGCACATCTTTCGCGCGACCTCTACTCAATGACTCAAGAATTA
GAGGTGAACTATTGCTTACACTATCTACTACTAAACATTGTAAAGTGACT
GCCAGTTCTATGAGACGTTCGTATAGCATGATGCATGATCATGAGAAAAG
CTAAAAGATACAGCGCAGAAAGAGCCAGAAGCTCGTTTCTAAAGGCAAC
GAAAGTGAAGCTGACCATGATGCAGTTTTTGGGCAAATAATGAAAATGTG
TGGATCTGACAATGACTCGAATTGGCCTCGGGAGTCGAGCACAAGTCCAA
GACCAAAAGAGGCTGCAAATCTGAACTCAAATGGGAAGACAAAAGCAAA
TAGGGGGTCAGCAACGGATCCACAAAGTGTCTACGCACGGAAGAGAAGA
GAACGAATTAATGAACGGTTAAGAATACTACAGAGTCTGGTTCCTAATGG
TACAAAGGTTGATATAAGCACAATGCTTGAAGATGCTGTCCAGTATGTGA
AATTTTTGCAGCTCCAAATCAAGCCGTTGAGCTCTGATGATCTGTGGATGT
ATGCCCCCATCGCGTACAACGGGATGGAGACGGGGCTTGATTCTACGATC
CCCTCGCCAAGGTGAAGACTATCCAAAGTTGCCGCATCTTTTTTCTTGAAA
AAAGGGAAGCCTGGGGCAA
BdRSLa amino acid sequence (SEQ ID NO: 100)
MALVREPMVLYDGGFDASEASAFDSIGCFGHGHGHDALLGGVDAAALFGG
YAHDEPAGASASAYVKDGSHWAGVGASVLAFDRAARGHGAQAMATAAAQ
EEEECDAWIDAMDEDNGEAAPAPSIGFDPATGCFSLTQRPGAGARRPFGLLFP
SASGGAPSPDSAAPAPASRGSQKRPSAGIARAQDAEPRASKKQCGASRKTTA
KAKSPAPAITSPKDPQSLAAKNRREKISERLRTLQEMVPNGTKVDMVTMLEK
AISYVKFLQLQVKVLATDEFWPAQGGMAPEISQVKEALDAILSSQRGQFNCS
S*
BdRSLa nucleotide sequence (SEQ ID NO: 101)
ATGGCATTAGTGCGGGAGCCGATGGTACTGTATGACGGCGGTTTCGACGC
CTCGGAGGCGTCGGCATTCGACTCCATCGGCTGCTTCGGCCACGGCCACG
GCCACGACGCGCTCCTAGGCGGCGTCGACGCGGCCGCGCTGTTCGGGGGC
TACGCGCACGACGAGCCGGCCGGCGCCAGCGCCAGCGCCTACGTGAAGG
ACGGCTCGCACTGGGCCGGCGTGGGTGCGTCCGTGCTCGCGTTCGACCGT
GCCGCTCGGGGCCACGGCGCGCAGGCCATGGCGACCGCGGCCGCTCAGG
AGGAGGAAGAATGCGACGCGTGGATCGACGCCATGGACGAGGACAATGG
CGAGGCGGCGCCGGCGCCGTCCATCGGCTTCGACCCGGCCACGGGCTGCT
TCAGCCTCACGCAGCGGCCCGGCGCCGGCGCGCGGCGCCCGTTCGGGCTC
CTGTTCCCGAGCGCGTCCGGTGGCGCGCCCTCGCCCGACAGCGCCGCGCC
AGCGCCGGCATCCCGCGGTTCCCAGAAGCGGCCATCCGCCGGGATTGCGC
GCGCGCAGGACGCGGAGCCGCGGGCCAGCAAGAAGCAGTGCGGCGCGAG
CAGGAAGACGACGGCCAAGGCGAAGTCGCCTGCGCCTGCCATCACCTCG
CCCAAGGACCCGCAGAGCCTCGCTGCAAAGAACCGGAGGGAGAAGATCA
GCGAGCGGCTCCGGACGTTGCAGGAGATGGTGCCCAACGGCACCAAGGT
GGACATGGTCACCATGCTCGAGAAGGCCATCAGCTACGTCAAGTTCCTGC
AGCTGCAAGTCAAGGTGCTCGCGACGGACGAGTTCTGGCCGGCGCAGGG
AGGGATGGCGCCGGAGATCTCCCAGGTGAAGGAGGCGCTCGACGCCATC
CTGTCGTCGCAGAGGGGGCAATTCAACTGCTCCAGCTAG
BdRSLb amino acid sequence (SEQ ID NO: 102)
MASRHATTREPHLRTMYDDEPSMSLELFGYHGVVVDGDDDGDTATDLPQLT
FVDNFKGGCGSADYYGWAYSASGGASGACSSSSSSVLSFEQAGGAGHQLAY
NAGTGDDDCALWMDGMADQHDTAKFGFMDPGMSDVSLEIQESSMKPPAK
MAQKRACQGGETQAAAKKQCGGSKKSKAKAAPAKDPQSAVAKVRRERISE
RLKVLQDLVPNGTKVDMVTMLEKAITYVKFLQLQVKVLATDDFWPVQGGK
APELSQVKDALDAILSSQNQS*
BdRSLb nucleotide sequence (SEQ ID NO: 103)
ATGGCAAGCAGGCACGCCACTACACGGGAGCCACACCTCCGGACCATGT
ACGACGACGAGCCATCCATGTCCCTCGAGCTCTTCGGCTACCATGGCGTC
GTCGTCGACGGTGACGACGATGGCGACACCGCCACCGACCTTCCCCAGCT
CACCTTTGTTGACAACTTCAAAGGCGGGTGTGGGTCAGCCGACTACTACG
GCTGGGCGTACAGCGCCTCCGGTGGTGCGTCAGGCGCCTGCTCCAGCTCC
AGCTCGTCGGTGCTCAGCTTTGAGCAGGCGGGTGGTGCCGGTCATCAGCT
GGCTTATAACGCCGGCACAGGTGACGATGACTGCGCGCTCTGGATGGACG
GCATGGCTGACCAGCATGACACAGCCAAGTTTGGGTTCATGGACCCAGGC
ATGTCTGATGTCAGCCTAGAAATCCAGGAGAGCAGCATGAAACCGCCGG
CCAAGATGGCACAGAAGCGCGCTTGCCAGGGTGGTGAGACGCAAGCAGC
GGCGAAGAAGCAGTGTGGAGGAAGCAAGAAGAGCAAGGCAAAAGCTGC
CCCTGCCAAGGATCCTCAAAGCGCCGTTGCAAAGGTCCGAAGAGAGCGC
ATCAGCGAGAGGCTCAAAGTTCTGCAGGATCTCGTGCCCAATGGCACAAA
GGTTGACATGGTCACCATGCTCGAAAAGGCAATCACCTATGTCAAGTTCC
TGCAGCTGCAAGTCAAGGTATTGGCGACTGATGACTTCTGGCCGGTGCAA
GGAGGGAAAGCTCCGGAGCTCTCCCAAGTGAAGGACGCTCTGGACGCGA
TCCTGTCTTCCCAGAATCAATCCTAG
BdRS Lc amino acid sequence (SEQ ID NO: 104)
MALVGQATKLCYDGFAGDGVPPFMDAACLAFDHGYDYNNPHAWEFPTGAE
PGNSSAFDVAWTGVSSTSPVLTFDAAEWMDATATDRLSSYSPSAATVPASYK
RPRAHVQPQQEAEEQESITPNPKKQCGDGKVVIKSSAAATGTSPRKEPQSQA
AKSRRERIGERLRALQELVPNGSKVDMVTMLDKAITYVKFMQLQLTVLETD
AFWPAQGGAAPEISQVKAALDAIILSSSQKPRQWS*
BdRS Lc nucleotide sequence (SEQ ID NO: 105)
ATGGCTCTAGTGGGTCAGGCAACGAAGCTCTGCTACGACGGCTTCGCCGG
AGACGGTGTGCCGCCGTTCATGGACGCAGCTTGTCTGGCATTCGACCACG
GGTATGATTACAACAATCCCCACGCATGGGAATTCCCCACCGGCGCCGAG
CCAGGCAACAGCAGCGCGTTCGACGTTGCCTGGACCGGCGTCTCCTCCAC
TTCTCCGGTGCTCACATTCGACGCCGCCGAGTGGATGGACGCCACGGCCA
CGGACCGGCTGAGCTCCTACAGCCCGTCTGCGGCCACCGTGCCGGCCTCT
TACAAGCGGCCTCGTGCGCACGTGCAGCCACAGCAGGAAGCAGAAGAAC
AGGAAAGCATTACTCCCAATCCCAAGAAGCAGTGCGGCGATGGGAAAGT
AGTTATCAAGTCATCGGCGGCGGCTACCGGCACCAGTCCACGCAAGGAAC
CCCAAAGCCAAGCTGCCAAGAGCCGTCGTGAGCGGATCGGCGAGCGGCT
GAGAGCGCTGCAGGAGCTGGTGCCCAACGGCAGCAAGGTGGACATGGTC
ACCATGCTCGACAAGGCCATCACTTATGTCAAGTTCATGCAGCTCCAGCT
CACGGTGCTCGAGACAGACGCGTTCTGGCCTGCGCAGGGTGGCGCGGCGC
CGGAGATCTCCCAGGTGAAGGCGGCGCTCGACGCCATCATCCTCTCCTCG
TCGCAGAAGCCTCGTCAGTGGAGCTAG
BdRSLd amino acid sequence (SEQ ID NO: 106)
MEAGGLISEAGWTMFDFPSQGEESEIMSQLLGAFPSHLEEGHQDLPWYQASD
PSYYDCNLNTSSESNASSLAVPSECMGYYLGDSSESLDLSSCIAPNDLNLVQE
QDATEFLNMTPNLSLDLRGNGESSCEDLTSVGPTNKRKHSSAEEGIDCQARG
QKFARKAEPKRTKKTKQSGWEVAVATRNGSTASCCTSDDDSNASQESADTG
VCPKGKARAARGASTDPQSLYARKRRERINERLKTLQTLVPNGTKVDMSTM
LEEAVHYVKFLQLQIKVLSSDDMWMYAPLAYNGMNIGLDLNIYTPERWRTA
SAAPSTEGREYAGVDRISDLPDGILGDIVSLLPTAEGARTQILKRRWRHIWRC
SAPLNLDCCTLVARGGGREAEDELVGLIPSILSSHQGTGRRFHVPSSRHSDRA
ATIEAWLQSAALDNLQELDLWCTHTYLYDYVPLPPAVFRFSATVRVVTIANC
NLRDSAVQGLQFPQLKQLGFKDIIIMEDSLHHMIAACPDLECLMIERSLGFAC
VRINSLSLRSIGVSTDHPHPHELQFVELVIDNAPCLKRLLHLEMCYHLDMHIT
VISAPKLETLSCCSSVSRSSTKLSFGSAAIQGLHIDSLTTVVRTVQILAVEMHSL
CLDTIIDFMKCFPCLQKLYIKSFVSGNNWWQRKHRNVIKSLDIRLKTIALESY
GGNQSDINFVTFFVLNARVLELMTFDVCSEHYTVEFLAEQYRKLQLDKRASR
AARFHFTSNRCVRGIPYIGRAELFLPIKCSHVDTSPNLSSFRLSAVFSVCITRNL
LRLKKAMWVISLYYSPEFTKQVAVHNPNEMPF*
BdRSLd nucleotide sequence (SEQ ID NO: 107)
ATGGAGGCTGGAGGGCTGATTTCTGAGGCTGGCTGGACCATGTTTGACTT
CCCGTCGCAAGGCGAGGAATCAGAGATCATGTCGCAGCTGCTAGGCGCCT
TCCCCTCCCATCTTGAGGAAGGCCATCAGGATCTGCCTTGGTACCAGGCTT
CTGACCCATCCTACTATGACTGTAATCTTAATACAAGTAGTGAAAGCAAT
GCTAGTAGTCTTGCTGTTCCATCCGAGTGTATGGGCTACTATTTGGGTGAT
TCAAGTGAGTCCCTGGACCTGAGCTCCTGCATTGCACCAAATGACCTGAA
CTTGGTCCAGGAGCAAGATGCAACTGAGTTTCTGAATATGACACCAAATC
TTTCCCTTGATTTACGTGGGAATGGTGAGTCGAGCTGCGAGGATCTCACTT
CGGTCGGTCCTACTAACAAGCGAAAGCACTCCTCGGCAGAAGAAGGAAT
CGACTGCCAAGCAAGAGGCCAGAAATTCGCCAGAAAGGCTGAACCGAAG
CGAACAAAGAAGACCAAGCAAAGCGGATGGGAGGTTGCTGTTGCCACCA
GGAATGGAAGCACAGCGAGCTGCTGCACCTCTGATGATGACTCAAACGCT
TCTCAAGAATCTGCAGATACCGGTGTTTGTCCGAAAGGCAAGGCTCGGGC
TGCCCGTGGCGCATCAACTGATCCCCAGAGCCTCTATGCAAGGAAAAGGA
GGGAAAGGATCAATGAGAGACTGAAGACACTGCAGACCCTTGTGCCCAA
TGGAACCAAAGTAGATATGAGCACCATGCTTGAGGAGGCAGTCCACTACG
TGAAGTTCCTGCAGCTTCAGATCAAGGTCTTGAGCTCTGATGATATGTGG
ATGTATGCGCCGCTAGCATACAACGGGATGAACATTGGGCTTGATCTGAA
CATATATACTCCGGAGAGGTGGAGGACAGCGTCCGCGGCGCCCTCAACCG
AAGGGCGTGAATACGCCGGCGTCGACCGCATCAGCGACCTCCCCGACGG
CATCCTCGGCGACATCGTCTCGTTGCTCCCCACCGCCGAAGGAGCCCGCA
CCCAGATCCTCAAGCGCAGGTGGCGCCACATCTGGCGCTGCTCCGCCCCT
CTCAACCTCGATTGCTGTACCTTGGTCGCCCGTGGCGGCGGCCGTGAGGC
TGAAGATGAACTCGTCGGTCTCATACCGTCCATCCTTTCTTCTCACCAAGG
CACCGGCCGCCGCTTCCACGTCCCCTCGTCGCGCCACTCTGACCGAGCTG
CTACCATTGAAGCCTGGCTCCAATCTGCTGCCCTCGACAATCTCCAGGAG
CTCGATTTATGGTGCACCCACACCTATCTTTACGACTATGTTCCGCTGCCA
CCCGCCGTCTTTCGCTTCTCCGCCACCGTCCGTGTTGTCACCATCGCAAAT
TGTAACCTCCGTGACAGCGCCGTCCAAGGCCTTCAATTCCCACAACTTAA
ACAGCTCGGATTCAAAGATATCATCATCATGGAGGATTCGCTGCACCACA
TGATTGCTGCGTGTCCAGATCTCGAGTGCTTGATGATTGAAAGGAGCTTA
GGTTTTGCTTGCGTCCGGATCAATTCCCTTAGTCTTAGAAGCATCGGTGTG
AGCACTGACCACCCTCACCCACATGAGCTCCAGTTTGTGGAACTCGTCAT
TGATAATGCACCTTGTCTTAAGAGATTGCTCCATCTTGAAATGTGTTATCA
CCTTGACATGCATATAACAGTAATCTCCGCGCCTAAACTGGAGACCTTGA
GCTGCTGTTCTTCTGTGAGTCGCTCCTCCACCAAACTCTCGTTTGGCTCCG
CGGCCATTCAGGGATTGCACATTGATAGCCTAACAACAGTGGTGCGCACT
GTCCAAATTTTAGCTGTAGAGATGCATTCTCTTTGTCTAGACACAATTATT
GACTTCATGAAATGCTTTCCATGTCTGCAGAAGTTGTACATTAAGTCATTT
GTAAGTGGAAACAATTGGTGGCAACGTAAACACCGGAACGTTATCAAATC
CCTTGACATCCGTCTCAAGACAATAGCGTTGGAAAGTTATGGGGGCAATC
AGTCTGACATCAACTTTGTCACATTCTTTGTCTTGAACGCGAGAGTGCTAG
AGTTGATGACATTTGACGTTTGTTCTGAGCATTACACTGTGGAGTTCTTGG
CAGAGCAATATAGGAAGCTTCAGCTAGATAAGAGGGCTTCAAGAGCCGC
TCGGTTCCATTTTACAAGTAACCGATGTGTCCGTGGTATTCCGTATATCGG
ACGTGCCGAGCTATTCTTGCCTATCAAATGTTCTCATGTTGACACCAGTCC
AAACTTGAGTAGTTTCCGTTTGTCTGCAGTATTTTCAGTTTGTATTACCCG
GAACCTTTTGCGTTTAAAAAAAGCTATGTGGGTCATTAGTTTGTATTATTC
TCCAGAATTTACAAAACAAGTGGCCGTGCACAATCCCAATGAAATGCCGT
TTTAG
BdRS Le amino acid sequence (SEQ ID NO: 108)
MEAKCGAIWSSIDARSEDSEMIAHLQSMFWSNSDVALNLCSSNTSGNSCVTA
STLPSSLFLPLVDNESYGAAPSVDTGMDSCFDHQHQSITGHKRISHMDEQMK
KTRKKSRTVPSVSKALGSSLVDNQMNADIFNQSSSCCSSGEDSIGTSEKSIVA
NQSDNTSGCKRPSKNMQSLYAKKRRERINEKLRVLQQLIPNGTKVDISTMLE
EAVQYVKFLQLQIKVLSSDETWMYAPLAYNGMDIGLTLALRTAANQE*
BdRS Le nucleotide sequence (SEQ ID NO: 109)
ATGGAGGCCAAGTGTGGAGCTATTTGGAGCTCTATCGATGCGAGGAGCGA
GGACTCTGAGATGATTGCTCACCTGCAGTCCATGTTCTGGAGCAACAGTG
ATGTTGCTCTCAACCTCTGTTCGTCAAACACCAGTGGCAATTCTTGTGTCA
CAGCTAGCACATTGCCTAGCAGCTTGTTCCTTCCTCTTGTCGATAATGAGA
GCTATGGTGCAGCGCCATCGGTGGACACCGGCATGGATTCATGCTTTGAT
CACCAGCATCAGAGCATTACTGGTCACAAGAGGATATCGCACATGGATGA
GCAGATGAAGAAGACGAGAAAGAAGTCCCGGACTGTTCCATCGGTATCA
AAGGCTCTGGGTTCCAGCCTAGTCGATAATCAGATGAATGCTGACATTTT
CAATCAGAGCTCCTCCTGCTGCAGCTCGGGAGAAGATTCAATTGGAACAT
CTGAGAAATCCATTGTTGCAAACCAGAGTGACAATACGAGTGGTTGTAAG
CGGCCTTCAAAGAATATGCAAAGCCTTTATGCAAAGAAGAGAAGAGAGA
GGATCAACGAGAAGTTGAGAGTACTGCAGCAGCTGATTCCCAATGGCACC
AAAGTTGACATCAGCACAATGTTGGAGGAAGCAGTTCAGTATGTCAAGTT
TCTGCAGCTGCAAATAAAGGTCTTAAGCTCTGACGAGACATGGATGTATG
CGCCCCTCGCCTACAATGGTATGGACATCGGTCTCACTCTCGCTCTGAGA
ACTGCTGCAAACCAAGAGTGA
Zea mays ZmRSLa amino acid sequence (AZM4_60871: SEQ ID NO: 110)
MALVREHGGYYGGFDSVEAAAFDTLGYGHGASLGFDASSALFGEGGYAAG
GGDAWAGAGASTVLAFNRTTAAAAVGVEEEEEECDAWIDAMDEDDQSSGP
AAAAPEARHALTASVGFDASTGCFTLTERASSSSGGAGRPFGLLFPSTSSSGG
TPERTAPVRVPQKRTYQAVSPNKKHCGAGRKASKAKLASTAPTKDPQSLAA
KQNRRERISERLRALQELVPNGTKVDLVTMLEKAISYVKFLQLQVKVLATDE
FWPAQGGKAPEISQVREALDAILSSAS
Zea mays ZmRSLa nucleotide sequence (AZM4_60871: SEQ ID NO: 111)
ATGGCGTTGGTGAGGGAGCACGGTGGGTACTACGGAGGCTTCGACAGCGT
CGAGGCGGCGGCCTTCGACACGCTCGGCTACGGCCACGGCGCGTCGCTGG
GCTTTGACGCGTCGTCGGCGCTGTTCGGGGAAGGCGGTTATGCGGCGGGC
GGCGGGGACGCCTGGGCGGGCGCGGGGGCGTCGACCGTCCTGGCGTTCA
ACCGCACAACGGCAGCGGCGGCCGTGGGTGTGGAAGAGGAGGAGGAGG
AGTGCGACGCGTGGATCGACGCTATGGACGAGGACGACCAGAGCTCCGG
CCCCGCCGCGGCGGCGCCAGAGGCGCGCCACGCGCTGACGGCCTCCGTG
GGTTTCGACGCCTCCACGGGGTGCTTCACCCTGACGGAGAGGGCGTCGTC
GTCGTCAGGCGGAGCGGGGCGCCCGTTCGGCCTGCTGTTCCCGAGCACGT
CGTCGTCGGGCGGCACGCCCGAGCGCACGGCGCCGGTGCGCGTCCCGCA
GAAACGGACCTACCAGGCTGTGAGCCCCAACAAGAAGCACTGCGGCGCG
GGCAGGAAGGCGAGCAAGGCCAAGCTCGCGTCCACAGCCCCAACCAAAG
ATCCCCAGAGCCTCGCGGCCAAGCAGAACCGGCGCGAGCGGATCAGCGA
GCGGCTGCGGGCGCTGCAGGAGCTGGTGCCCAACGGCACCAAGGTCGAC
CTGGTCACCATGCTCGAGAAGGCCATCAGCTACGTTAAGTTCCTCCAGTT
GCAAGTCAAGGTTCTGGCAACAGACGAATTCTGGCCGGCACAGGGAGGG
AAGGCGCCGGAGATCTCCCAGGTGAGGGAGGCGCTCGACGCCATCTTGTC
GTCGGCGTCG
Zea mays ZmRSLb amino acid sequence (AZM4_70092: SEQ ID NO: 112)
MAQFLGAADDHCFTYEYEHVDESMEAIAALFLPTLDTDSANFSSSCFNYAVP
PQCWPQPDHSSSVTSLLDPAENFEFPVRDPLPPSGFDPHCAVAYLTEDSSPLH
GKRSSVIEEEAANAAPAAKKRKAGAAMQGSKKSRKASKKDNIGDADDDGG
YACVDTQSSSSCTSEDGNFEGNTNSSSKKTCARASRGAATEPQSLYARKRRE
RINERLRILQNLVPNGTKVDISTMLEEAAQYVKFLQLQIKLLSCDDTWMYAPI
AYNGINIGNVDLNIYSLQK*
Zea mays ZmRSLb nucleotide sequence (AZM4_70092: SEQ ID NO: 113)
ATGGCTCAGTTTCTTGGGGCGGCTGATGATCACTGCTTCACCTACGAGTAT
GAGCATGTGGATGAGTCCATGGAAGCAATAGCAGCCCTGTTCTTGCCTAC
CCTTGACACCGACTCCGCCAACTTCTCCTCTAGCTGTTTCAACTATGCTGT
CCCTCCACAGTGCTGGCCTCAGCCAGACCATAGCTCTAGCGTTACCAGTTT
GCTTGATCCAGCCGAGAACTTTGAGTTTCCAGTCAGGGACCCGCTCCCCC
CAAGCGGCTTCGATCCACATTGCGCTGTCGCCTACCTCACTGAGGATTCG
AGCCCTCTGCATGGCAAACGTTCATCAGTCATTGAGGAAGAAGCAGCCAA
CGCCGCACCTGCTGCTAAGAAGAGGAAGGCTGGTGCTGCAATGCAGGGA
TCAAAGAAATCCAGGAAGGCGAGCAAAAAGGATAACATCGGCGACGCCG
ACGATGATGGCGGCTATGCCTGTGTTGACACGCAAAGCTCCAGTAGCTGC
ACCTCCGAGGACGGGAACTTCGAAGGAAATACGAATTCAAGCTCCAAGA
AGACCTGCGCCAGGGCCAGCCGCGGAGCAGCAACTGAACCTCAGAGTCT
CTATGCAAGGAAGAGGAGAGAGAGGATCAACGAAAGGTTGAGAATCTTG
CAGAACTTGGTTCCAAATGGAACAAAAGTAGACATTAGCACGATGCTCGA
GGAAGCGGCGCAGTATGTCAAGTTTTTACAGCTCCAGATTAAGCTGTTGA
GCTGTGACGACACATGGATGTATGCGCCAATCGCGTACAATGGAATTAAC
ATCGGCAATGTTGATCTGAACATCTACTCTCTGCAAAAGTAA
Zea mays ZmRSLc amino acid sequence (AZM4_91750: SEQ ID NO: 114)
MEDGGLXSEAGAWAELGTGGDESEELVAQLLGAFFRSHGEEGRHQLLWSD
DQASSDDVHGDGSLAVPLAYDGCCGYLSYSGSNSDELPLGSSSRAAPAGGPP
EELLGAAETEYLNNVAAADHPFFKWCGNGEGLDGPTSVVGTLGLGSGRKRA
RKKSGDEDEDPSTAIASGSGPTSCCTTSDSDSNASPLESADAGARRPKGNENA
RAAGRGAAAATTTTAEPQSIYARVRRERINERLKVLQSLVPNGTKVDMSTML
EEAVHYVKFLQLQIRVLQLLSSDDTWMYAPIAYNGMGIGIDLRMHGQDR*
Zea mays ZmRSLc nucleotide sequence (AZM4_91750: SEQ ID NO: 115)
ATGGAGGACGGAGGGTTGRTCAGCGAGGCCGGCGCCTGGGCCGAGCTCG
GCACCGGCGGCGACGAGTCGGAGGAGCTGGTGGCGCAGCTGCTGGGCGC
CTTCTTCCGGTCCCACGGCGAGGAAGGCCGGCACCAGCTGCTTTGGTCTG
ACGACCAAGCTTCTTCCGACGACGTGCACGGCGACGGCAGCCTTGCCGTG
CCGCTCGCATACGACGGCTGCTGCGGCTATCTGAGCTACTCAGGTAGCAA
CTCGGACGAGCTCCCCCTCGGGAGCAGCTCCCGCGCTGCGCCAGCAGGTG
GCCCACCGGAGGAGCTGCTCGGTGCAGCTGAGACTGAGTACCTGAATAAT
GTGGCCGCCGCAGACCATCCCTTCTTCAAATGGTGTGGGAATGGTGAGGG
TCTGGATGGTCCGACGAGCGTCGTGGGCACGCTTGGGCTTGGCTCGGGCC
GGAAACGCGCGCGCAAGAAGAGCGGGGACGAAGACGAAGACCCGAGCA
CGGCCATCGCCAGCGGAAGCGGCCCCACGAGCTGCTGCACTACCTCCGAC
AGCGACTCAAACGCGTCTCCTCTGGAGTCCGCGGACGCCGGCGCTCGTCG
CCCCAAGGGCAACGAGAATGCCCGGGCAGCTGGCCGCGGCGCGGCGGCG
GCGACGACGACGACAGCGGAGCCCCAGAGCATCTACGCAAGGGTACGGA
GGGAGCGGATCAACGAGAGGCTCAAGGTGCTGCAGAGCCTGGTGCCCAA
CGGCACCAAGGTGGACATGAGCACCATGCTCGAGGAGGCCGTCCACTAC
GTCAAGTTCCTGCAGCTTCAGATCAGGGTGCTGCAGCTCCTGAGCTCCGA
CGACACGTGGATGTACGCGCCCATCGCGTACAACGGGATGGGCATCGGG
ATCGACCTCCGCATGCATGGACAGGACAGATGA
Zea mays amino acid sequence (AZM4_86104: SEQ ID NO: 116)
SKKSRKASKKDCIVDDDDVYVDPQSSGSCTSEEGNFEGNTYSSAKKTCTRAS
RGGATDPQSLYARKRRERINERLRILQNLVPNGTKVDISTMLEEAAQYVKFL
QLQIKLLSSDDMWMYAPIAYNGINISNVDLNIPALQK*
Zea mays ZmRSLd nucleotide sequence (AZM4_86104: SEQ ID NO: 117)
TCAAAGAAATCCAGGAAGGCGAGCAAAAAAGATTGTATTGTCGATGACG
ACGATGTCTATGTTGACCCGCAAAGCTCCGGTAGCTGCACCTCCGAGGAG
GGGAATTTTGAAGGGAATACGTATTCAAGCGCGAAAAAGACCTGCACCA
GGGCCAGCCGCGGAGGAGCAACTGATCCTCAGAGTCTCTATGCAAGGAA
GAGGAGAGAGAGGATCAATGAAAGGTTGAGAATCTTGCAGAACTTGGTC
CCCAATGGAACAAAGGTTGACATTAGTACGATGCTCGAGGAAGCAGCAC
AGTATGTCAAATTTTTACAGCTTCAGATTAAGCTGTTGAGCTCTGACGACA
TGTGGATGTATGCGCCAATCGCGTACAATGGGATCAACATCAGCAATGTT
GATCTGAACATCCCTGCA

The invention is further described by the following numbered paragraphs:

1. An expression construct for constitutive expression of a plant transcription factor gene comprising an isolated plant nucleic acid sequence encoding a transcription factor operably linked to an isolated plant promoter nucleic acid sequence wherein said promoter sequence is derived from the promoter sequence of a target gene of said transcription factor and wherein said transcription factor regulates expression of said target gene.

2. An expression construct according to paragraph 1 wherein said promoter is a cell, tissue or organ specific promoter.

3. An expression construct according to paragraph 2 wherein said promoter is a root specific promoter.

4. An expression construct according to paragraph 3 wherein said promoter is EXP7.

5. An expression construct according to a preceding paragraph wherein said transcription factor is RSL4 or a functional homolog or ortholog thereof.

6. An expression construct according to any of paragraphs 1 to 4 wherein said transcription factor is selected from transcription factors listed in table 1.

7. An expression construct according to a preceding paragraph wherein said plant is a crop plant.

8. A vector comprising an expression construct according to any of paragraphs 1 to 7.

9. A vector according to paragraph 8 further comprising a second expression construct comprising an isolated plant nucleic acid sequence encoding said transcription factor operably linked to a second isolated plant promoter nucleic acid sequence specific to a cell, tissue or organ in which said transcription factor is not normally expressed.

10. A vector according to paragraph 9 wherein the first promoter is EXP7.

11. A vector according to paragraph 8 or 9 wherein said transcription factor is RSL4 or a functional variant thereof.

12. A vector according to any of paragraphs 8 to 10 wherein said second promoter is GL2.

13. A host cell comprising an expression construct according to any of paragraphs 1 to 6 or a vector according to any of paragraphs 8 to 12.

14. A host cell according to paragraph 13 wherein said host cell is a plant cell.

15. A plant expressing a expression construct according to any of paragraphs 1 to 7 or a vector according to any of paragraphs 8 to 12.

16. A method for constitutive expression of a plant transcription factor gene comprising introducing the expression construct according to any of paragraphs 1 to 7 or vector according to any of paragraphs 8 to 12 into a plant host cell or plant expressing the transcription factor gene.

17. A method according to paragraph 16 comprising introducing the expression construct according to any of paragraphs 1 to 7 or vector according to any of paragraph 8 into a plant host cell or plant wherein said transcription factor gene is constitutively expressed in a cell or tissue in which it is normally expressed.

18. A method according to any of paragraph 16 comprising introducing a vector according to paragraph 9 to 12 into a host cell or organism wherein said transcription factor gene is constitutively expressed in a cell or tissue in which it is not normally expressed.

19. A method according to any of paragraphs 16 to 18 comprising introducing the expression construct according to any of paragraphs 1 to 7 and a second expression construct into said host cell or organism wherein said second expression construct comprises an isolated nucleic acid sequence encoding said transcription factor operably linked to a second isolated promoter nucleic acid sequence specific to a cell, tissue or organ in which said transcription factor is not normally expressed.

20. A method for expression of a plant transcription factor in a tissue in which it is not normally expressed said method comprising introducing the vector of any of paragraphs 9 to 12 into a plant host cell or plant.

21. A composition comprising an expression construct for constitutive expression of a plant transcription factor gene comprising an isolated plant nucleic acid sequence encoding a transcription factor operably linked to an isolated plant promoter nucleic acid sequence wherein said promoter sequence is derived from the promoter sequence of a target gene of said transcription factor and wherein said transcription factor regulates expression of said target gene.

22. A composition according to paragraph 21 further comprising a second expression construct comprising an isolated plant nucleic acid sequence encoding said transcription factor operably linked to a second isolated plant promoter nucleic acid sequence specific to a cell, tissue or organ in which said transcription factor is not normally expressed.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An expression construct for constitutive expression of a plant transcription factor gene comprising an isolated plant nucleic acid sequence encoding a transcription factor operably linked to an isolated plant promoter nucleic acid sequence wherein said promoter sequence is derived from the promoter sequence of a target gene of said transcription factor and wherein said transcription factor regulates expression of said target gene.

2. An expression construct according to claim 1 wherein said promoter is a cell, tissue or organ specific promoter.

3. An expression construct according to claim 2 wherein said promoter is a root specific promoter.

4. An expression construct according to claim 3 wherein said promoter is EXP7.

5. An expression construct according to a preceding claim wherein said transcription factor is RSL4 or a functional homolog or ortholog thereof.

6. An expression construct according to claim 1 wherein said transcription factor is selected from transcription factors listed in table 1.

7. An expression construct according to claim 1 wherein said plant is a crop plant.

8. A vector comprising an expression construct according to claim 1.

9. A vector according to claim 8 further comprising a second expression construct comprising an isolated plant nucleic acid sequence encoding said transcription factor operably linked to a second isolated plant promoter nucleic acid sequence specific to a cell, tissue or organ in which said transcription factor is not normally expressed.

10. A vector according to claim 9 wherein the first promoter is EXP7.

11. A vector according to claim 8 wherein said transcription factor is RSL4 or a functional variant thereof.

12. A vector according to claim 8 wherein said second promoter is GL2.

13. A host cell comprising an expression construct according to any of claims 1 to 6 or a vector according to claim 8.

14. A host cell according to claim 13 wherein said host cell is a plant cell.

15. A plant expressing a expression construct according to claim 1.

16. A method for constitutive expression of a plant transcription factor gene comprising introducing the expression construct according to claim 1 into a plant host cell or plant expressing the transcription factor gene.

17. A method according to claim 16 comprising introducing the expression construct into a plant host cell or plant wherein said transcription factor gene is constitutively expressed in a cell or tissue in which it is normally expressed.

18. A method according to claim 16 comprising introducing the expression construct and a second expression construct into said host cell or organism wherein said second expression construct comprises an isolated nucleic acid sequence encoding said transcription factor operably linked to a second isolated promoter nucleic acid sequence specific to a cell, tissue or organ in which said transcription factor is not normally expressed.

19. A method for expression of a plant transcription factor in a tissue in which it is not normally expressed said method comprising introducing the vector of claim 9 into a plant host cell or plant.

20. A composition comprising an expression construct for constitutive expression of a plant transcription factor gene comprising an isolated plant nucleic acid sequence encoding a transcription factor operably linked to an isolated plant promoter nucleic acid sequence wherein said promoter sequence is derived from the promoter sequence of a target gene of said transcription factor and wherein said transcription factor regulates expression of said target gene.

21. A composition according to claim 20 further comprising a second expression construct comprising an isolated plant nucleic acid sequence encoding said transcription factor operably linked to a second isolated plant promoter nucleic acid sequence specific to a cell, tissue or organ in which said transcription factor is not normally expressed.
